SEQUENCE LISTING (122 sequences)

SEQ ID NO: 1 - skipped

SEQ ID NO: 2 - protein, 5 aa, synthetic construct
RAYAR

SEQ ID NO: 3 - protein, 5 aa, synthetic construct
KAYAK

SEQ ID NO: 4 - skipped

SEQ ID NO: 5 - protein, 7 aa, synthetic construct
  VARIANT 1: note = independently any amino acid
  REPEAT 1: note = repeat 0 to 20 times
  VARIANT 2: note = independently any positively charged amino acid
  VARIANT 3..5: note = independently any amino acid
  VARIANT 6: note = independently any positively charged amino acid
  VARIANT 7: note = independently any amino acid
  REPEAT 7: note = repeat 0 to 20 times
ZBXXXBZ

SEQ ID NO: 6 - protein, 29 aa, synthetic construct
QARILEADAEILRAYARILEAHAEILRAQ

SEQ ID NO: 7 - protein, 29 aa, synthetic construct
QAKILEADAEILKAYAKILEAHAEILKAQ

SEQ ID NO: 8 - protein, 23 aa, synthetic construct
ADAEILRAYARILEAHAEILRAQ

SEQ ID NO: 9 - protein, 29 aa, synthetic construct
  MOD_RES <1: note = N-terminal acetylation
  MOD_RES >29: note = C-terminal amidation
QARILEADAEILRAYARILEAHAEILRAQ

SEQ ID NO: 10 - protein, 29 aa, synthetic construct
  MOD_RES <1: note = N-terminal acetylation
  MOD_RES >29: note = C-terminal amidation
QAKILEADAEILKAYAKILEAHAEILKAQ

SEQ ID NO: 11 - protein, 23 aa, synthetic construct
  MOD_RES <1: note = N-terminal acetylation
  MOD_RES >23: note = C-terminal amidation
ADAEILRAYARILEAHAEILRAQ

SEQ ID NO: 12 - protein, 11 aa, synthetic construct
QQKFQFQFEQQ

SEQ ID NO: 13 - protein, 11 aa, synthetic construct
  MOD_RES <1: note = N-terminal acetylation
  MOD_RES >11: note = C-terminal amidation
QQKFQFQFEQQ

SEQ ID NO: 14 - protein, 8 aa, synthetic construct
FKFEFKFE

SEQ ID NO: 15 - protein, 7 aa, synthetic construct
KFQFQFE

SEQ ID NO: 16 - protein, 11 aa, synthetic construct
QQRFQFQFEQQ

SEQ ID NO: 17 - protein, 11 aa, synthetic construct
QQRFQWQFEQQ

SEQ ID NO: 18 - protein, 16 aa, synthetic construct
FEFEFKFKFEFEFKFK

SEQ ID NO: 19 - protein, 11 aa, synthetic construct
QQRFEWEFEQQ

SEQ ID NO: 20 - protein, 11 aa, synthetic construct
  SITE 3: note = ornithine
  SITE 5: note = ornithine
  SITE 7: note = ornithine
QQXFXWXFQQQ

SEQ ID NO: 21 - protein, 12 aa, synthetic construct
FKFEFKFEFKFE

SEQ ID NO: 22 - protein, 12 aa, synthetic construct
FKFQFKFQFKFQ

SEQ ID NO: 23 - protein, 16 aa, synthetic construct
AEAKAEAKAEAKAEAK

SEQ ID NO: 24 - protein, 16 aa, synthetic construct
AEAEAKAKAEAEAKAK

SEQ ID NO: 25 - protein, 16 aa, synthetic construct
AEAEAEAEAKAKAKAK

SEQ ID NO: 26 - protein, 16 aa, synthetic construct
RADARADARADARADA

SEQ ID NO: 27 - protein, 16 aa, synthetic construct
RARADADARARADADA

SEQ ID NO: 28 - protein, 33 aa, synthetic construct
SGRGYBLGGQGAGAAAAAGGAGQGGYGGLGSQG

SEQ ID NO: 29 - protein, 10 aa, synthetic construct
  VARIANT 4: note = Val, Ala, Ser, or Pro
  VARIANT 6: note = Val, Ala, Ser, or Pro
  VARIANT 8: note = Val, Ala, Ser, or Pro
  VARIANT 10: note = Val, Ala, Ser, or Pro
EWEXEXEXEX

SEQ ID NO: 30 - protein, 10 aa, synthetic construct
  VARIANT 3: note = Val, Ala, Ser, or Pro
  VARIANT 5: note = Val, Ala, Ser, or Pro
  VARIANT 7: note = Val, Ala, Ser, or Pro
  VARIANT 9: note = Val, Ala, Ser, or Pro
WKXKXKXKXK

SEQ ID NO: 31 - protein, 15 aa, synthetic construct
KWKVKVKVKVKVKVK

SEQ ID NO: 32 - protein, 16 aa, synthetic construct
LLLLKKKKKKKKLLLL

SEQ ID NO: 33 - protein, 21 aa, synthetic construct
VKVKVKVKVDPPTKVKVKVKV

SEQ ID NO: 34 - protein, 21 aa, synthetic construct
VKVKVKVKVDPPTKVKTKVKV

SEQ ID NO: 35 - protein, 21 aa, synthetic construct
KVKVKVKVKDPPSVKVKVKVK

SEQ ID NO: 36 - protein, 21 aa, synthetic construct
VKVKVKVKVDPPSKVKVKVKV

SEQ ID NO: 37 - protein, 21 aa, synthetic construct
VKVKVKTKVDPPTKVKTKVKV

SEQ ID NO: 38 - protein, 11 aa, synthetic construct
  VARIANT 5: note = Glu, Asp, or Asn
QQKFXFQFEQQ

SEQ ID NO: 39 - protein, 11 aa, synthetic construct
  VARIANT 6: note = Trp or Tyr
QQKFQXQFEQQ

SEQ ID NO: 40 - protein, 11 aa, synthetic construct
  VARIANT 7: note = Glu, Asp, or Asn
QQKFQFXFEQQ

SEQ ID NO: 41 - protein, 9 aa, synthetic construct
VEVKVEVKV

SEQ ID NO: 42 - protein, 12 aa, synthetic construct
VEVKVEVKVEVK

SEQ ID NO: 43 - protein, 9 aa, synthetic construct
VVVAAAEEE

SEQ ID NO: 44 - protein, 20 aa, synthetic construct
VEVEVEVEVEVEVEVEVEVE

SEQ ID NO: 45 - protein, 9 aa, synthetic construct
CGNKRTRGC

SEQ ID NO: 46 - protein, 21 aa, synthetic construct
VKVKVKVKVDPPTKVEVKVKV

SEQ ID NO: 47 - protein, 8 aa, synthetic construct
LRKKLGKA

SEQ ID NO: 48 - protein, 8 aa, synthetic construct
VVVVVVKK

SEQ ID NO: 49 - protein, 16 aa, synthetic construct
AEAKAEAKAEAKAEAK

SEQ ID NO: 50 - protein, 8 aa, synthetic construct
AEAKAEAK

SEQ ID NO: 51 - protein, 12 aa, synthetic construct
AEAEAEAEAKAK

SEQ ID NO: 52 - protein, 8 aa, synthetic construct
AEAEAKAK

SEQ ID NO: 53 - protein, 16 aa, synthetic construct
AEAEAKAKAEAEAKAK

SEQ ID NO: 54 - protein, 16 aa, synthetic construct
RADARADARADARADA

SEQ ID NO: 55 - protein, 16 aa, synthetic construct
RADARGDARADARGDA

SEQ ID NO: 56 - protein, 8 aa, synthetic construct
RADARADA

SEQ ID NO: 57 - protein, 16 aa, synthetic construct
RARADADARARADADA

SEQ ID NO: 58 - protein, 8 aa, synthetic construct
RARADADA

SEQ ID NO: 59 - protein, 16 aa, synthetic construct
RARARARADADADADA

SEQ ID NO: 60 - protein, 16 aa, synthetic construct
ADADADADARARARAR

SEQ ID NO: 61 - protein, 16 aa, synthetic construct
DADADADARARARARA

SEQ ID NO: 62 - protein, 16 aa, synthetic construct
RAEARAEARAEARAEA

SEQ ID NO: 63 - protein, 8 aa, synthetic construct
RAEARAEA

SEQ ID NO: 64 - protein, 16 aa, synthetic construct
KAKAKAKAEAEAEAEA

SEQ ID NO: 65 - protein, 16 aa, synthetic construct
AEAEAEAEAKAKAKAK

SEQ ID NO: 66 - protein, 16 aa, synthetic construct
KADAKADAKADAKADA

SEQ ID NO: 67 - protein, 8 aa, synthetic construct
KADAKADA

SEQ ID NO: 68 - protein, 17 aa, synthetic construct
AEAEAHAHAEAEAHAHA

SEQ ID NO: 69 - protein, 9 aa, synthetic construct
AEAEAHAHA

SEQ ID NO: 70 - protein, 16 aa, synthetic construct
HEHEHKHKHEHEHKHK

SEQ ID NO: 71 - protein, 8 aa, synthetic construct
HEHEHKHK

SEQ ID NO: 72 - protein, 16 aa, synthetic construct
FEFEFKFKFEFEFKFK

SEQ ID NO: 73 - protein, 8 aa, synthetic construct
FEFKFEFK

SEQ ID NO: 74 - protein, 16 aa, synthetic construct
LELELKLKLELELKLK

SEQ ID NO: 75 - protein, 8 aa, synthetic construct
LELELKLK

SEQ ID NO: 76 - protein, 12 aa, synthetic construct
KFDLKKDLKLDL

SEQ ID NO: 77 - protein, 8 aa, synthetic construct
FKFEFKFF

SEQ ID NO: 78 - protein, 8 aa, synthetic construct
FEFEFKFK

SEQ ID NO: 79 - protein, 20 aa, synthetic construct
RFRFRFRFRFRFRFRFRFRF

SEQ ID NO: 80-82 - skipped (3 entries)

SEQ ID NO: 83 - protein, 4 aa, synthetic construct
SGSG

SEQ ID NO: 84 - protein, 4 aa, synthetic construct
SGSG

SEQ ID NO: 85 - protein, 8 aa, synthetic construct
CCCCSGSG

SEQ ID NO: 86 - protein, 10 aa, synthetic construct
  REPEAT 2..10: note = may or may not be present
GGGGGGGGGG

SEQ ID NO: 87 - protein, 4 aa, synthetic construct
GSGS

SEQ ID NO: 88 - protein, 4 aa, synthetic construct
SSSS

SEQ ID NO: 89 - protein, 4 aa, synthetic construct
GGGS

SEQ ID NO: 90 - skipped

SEQ ID NO: 91 - protein, 24 aa, synthetic construct
GGCGGCGGCGGCGGCGGCGGCGGC

SEQ ID NO: 92 - protein, 15 aa, synthetic construct
GGGGSGGGGSGGGGS

SEQ ID NO: 93 - protein, 5 aa, synthetic construct
KSGSG

SEQ ID NO: 94 - protein, 6 aa, synthetic construct
KKSGSG

SEQ ID NO: 95 - protein, 10 aa, synthetic construct
EAAAKEAAAK

SEQ ID NO: 96 - protein, 5 aa, synthetic construct
GGAAY

SEQ ID NO: 97 - protein, 20 aa, synthetic construct
ASPAAPAPASPAAPAPSAPA

SEQ ID NO: 98 - protein, 20 aa, synthetic construct
  MOD_RES >20: note = C-terminal amidation
ASPAAPAPASPAAPAPSAPA

SEQ ID NO: 99 - protein, 12 aa, synthetic construct
  SITE 3: note = cyclohexylalanine
  SITE 1: note = D-alanine
  SITE 12: note = D-alanine
AKXVAAWTLKAA

SEQ ID NO: 100 - protein, 15 aa, synthetic construct
QLVFNSISARALKAY

SEQ ID NO: 101 - protein, 15 aa, synthetic construct
QAEILRAYARILEAQ

SEQ ID NO: 102 - protein, 22 aa, synthetic construct
QAEILRAYARILEAHAEILKAQ

SEQ ID NO: 103 - protein, 29 aa, synthetic construct
QAEILRAYARILEADAKILEAHAEILKAQ

SEQ ID NO: 104 - protein, 36 aa, synthetic construct
QAEILRAYARILEADAEILKAQAKILEAHAEILKAQ

SEQ ID NO: 105 - protein, 12 aa, synthetic construct
KESPHKPVQLGR

SEQ ID NO: 106 - protein, 16 aa, synthetic construct
EERVARVTIGPLCIRA

SEQ ID NO: 107 - protein, 11 aa, synthetic construct
DKGLRKCCEDG

SEQ ID NO: 108 - protein, 9 aa, synthetic construct
RYSCQRRAR

SEQ ID NO: 109 - protein, 13 aa, synthetic construct
IKAFIDCCNHITK

SEQ ID NO: 110 - protein, 12 aa, synthetic construct
  MOD_RES >12: note = C-terminal amidation
KESPHKPVQLGR

SEQ ID NO: 111 - protein, 16 aa, synthetic construct
  MOD_RES >16: note = C-terminal amidation
EERVARVTIGPLCIRA

SEQ ID NO: 112 - protein, 11 aa, synthetic construct
  MOD_RES >11: note = C-terminal amidation
DKGLRKCCEDG

SEQ ID NO: 113 - protein, 9 aa, synthetic construct
  MOD_RES >9: note = C-terminal amidation
RYSCQRRAR

SEQ ID NO: 114 - protein, 13 aa, synthetic construct
  MOD_RES >13: note = C-terminal amidation
IKAFIDCCNHITK

SEQ ID NO: 115 - protein, 59 aa, synthetic construct
  MOD_RES >59: note = C-terminal amidation
KESPHKPVQLGRSGSGEAAAKEAAAKSGSGQARILEADAEILRAYARILEAHAEILRAQ

SEQ ID NO: 116 - protein, 63 aa, synthetic construct
  MOD_RES >63: note = C-terminal amidation
EERVARVTIGPLCIRASGSGEAAAKEAAAKSGSGQARILEADAEILRAYARILEAHAEILRAQ

SEQ ID NO: 117 - protein, 58 aa, synthetic construct
  MOD_RES >58: note = C-terminal amidation
DKGLRKCCEDGSGSGEAAAKEAAAKSGSGQARILEADAEILRAYARILEAHAEILRAQ

SEQ ID NO: 118 - protein, 56 aa, synthetic construct
  MOD_RES >56: note = C-terminal amidation
RYSCQRRARSGSGEAAAKEAAAKSGSGQARILEADAEILRAYARILEAHAEILRAQ

SEQ ID NO: 119 - protein, 60 aa, synthetic construct
  MOD_RES >60: note = C-terminal amidation
IKAFIDCCNHITKSGSGEAAAKEAAAKSGSGQARILEADAEILRAYARILEAHAEILRAQ

SEQ ID NO: 120 - protein, 12 aa, synthetic construct
  SITE 1: note = D-alanine
  SITE 3: note = cyclohexylalanine
  SITE 12: note = D-alanine
  MOD_RES 12: note = C-terminal amidation
AKXVAAWTLKAA

SEQ ID NO: 121 - protein, 45 aa, synthetic construct
  SITE 1: note = D-alanine
  SITE 3: note = cyclohexylalanine
  SITE 12: note = D-alanine
  MOD_RES >45: note = C-terminal amidation
AKXVAAWTLKAASGSGQARILEADAEILRAYARILEAHAEILRAQ

SEQ ID NO: 122 - protein, 47 aa, synthetic construct
  MOD_RES >47: note = C-terminal amidation
SGSGEAAAKEAAAKSGSGQARILEADAEILRAYARILEAHAEILRAQ

(19) United States
(12) Patent Application Publication
(10) Pub. No.: US 2026/0325616 A1
(43) Pub. Date: Oct. 22, 2026

(54) COMPOSITIONS AND METHODS FOR RAISING MUCOSAL AND SYSTEMIC IMMUNE RESPONSES AGAINST COMPLEMENT ANAPHYLATOXINS FOR INFLAMMATORY DISEASE

(71) Applicant: Duke University, Durham, NC (US)

(72) Inventors: Joel Collier, Durham, NC (US); Kelly Hainline, Durham, NC (US); Helena Freire Haddad, Durham, NC (US)

(21) Appl. No.: 19/476,569

(22) PCT Filed: Apr. 18, 2024
(86) PCT No.: PCT/US2024/025292

§ 371 Date: Oct. 17, 2025

Related U.S. Application Data
(60) Provisional application No. 63/460,060, filed on Apr. 18, 2023.

(51) Int. Cl. A61K 39/00; A61K 9/00; A61K 39/385; A61K 39/39; A61P 37/04; C07K 14/47

(52) U.S. Cl. CPC A61K 39/0008; A61K 9/006; A61K 39/385; A61K 39/39; A61P 37/04; C07K 14/472; A61K 2039/605; A61K 2039/6093

(57) ABSTRACT

Embodiments are directed to an anaphylatoxin conjugate peptide including a self-assembling peptide and at least one anaphylatoxin epitope. Anaphylatoxin epitopes may include C3a1, C3a2, C3a3, C5al, and/or C5a3, as detailed herein. The anaphylatoxin conjugate peptide may self-assemble into a nanofiber or fibril. Compositions including the anaphylatoxin conjugate peptide may be used to treat inflammation and sepsis.

### CROSS-REFERENCE TO RELATED APPLICATIONS

This application claims priority to U.S. Provisional Patent Application No. 63/460,060, filed Apr. 18, 2023, which is incorporated herein by reference in its entirety.

### STATEMENT REGARDING FEDERALLY SPONSORED RESEARCH

This invention was made with government support under grant R01-AI172151-01 awarded by the National Institutes of Health (NIH) National Institute of Allergy and Infectious Diseases (NIAID). The government has certain rights in the invention.

### FIELD

Embodiments of this invention are directed generally to biology, medicine, and immunology. Certain aspects are directed to peptide conjugates and nanofibers and their use in inducing an immune response to treat a condition such as inflammation.

### INTRODUCTION

The complement system is a highly conserved part of innate immunity that is key to providing an adequate response to injury and infection. Upon activation through the classic, lectin or alternative pathways, a series of biochemical reactions result in the cleavage of the C3 and C5 convertases into the small effector proteins C3a and C5a, known as the complement anaphylatoxins. The complement anaphylatoxins C3a and C5a are terminal proteins in the complement cascade that play important roles in health and disease and have been linked to the pathology of several diseases, including but not limited to, sepsis and inflammatory bowel disease (IBD). In chronic inflammatory settings, both molecules play a pro-inflammatory role, making them interesting targets for an active immunotherapy, in which a patient's own immune system is stimulated to produced protective immune responses against problematic self-molecules as an alternative for monoclonal antibodies. While C5a has an unambiguous driving role in inflammation, C3a has been shown to have a dual role depending on its stage. Some functions of C3a include the inhibition of neutrophil migration and activation of macrophages. In acute inflammation when neutrophil responses are more prominent, C3a and C5a play opposing roles, with C5a acting as a classic pro-inflammatory mediator and C3a as an anti-inflammatory molecule. In chronic inflammation when macrophage responses are dominant, C3a has a pro-inflammatory effect. In chronic inflammatory conditions such as inflammatory bowel disease (IBD), both anaphylatoxins have been shown to play a pathogenic role and exacerbate inflammation. In sepsis, a model of acute inflammation, genetic blockade of C5aR ameliorated inflammation, while a C3aR knockout led to a worse disease outcome. This can be attributed to the dual role C3a plays in inflammation, inhibiting neutrophil mobilization during acute inflammation, but acting as a pro-inflammatory molecule on monocytes and macrophages during chronic inflammation. Immune therapies against C3a and C5a may be a translational alternative to interfere with the C3a/C3aR and C5a/C5aR axis, to study the role of both molecules in diverse stages of inflammation but also to apply in a therapeutic context. Generating safe, effective, and long-lasting immune responses across populations for treating inflammation is a major challenge.

### SUMMARY

In an aspect, provided herein is an anaphylatoxin conjugate peptide. The anaphylatoxin conjugate peptide may include (i) a self-assembling peptide comprising a polypeptide having the amino acid sequence of bXXXb (SEQ ID NO: 1, wherein X is independently any amino acid and b is independently any positively charged amino acid), QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and (ii) at least one anaphylatoxin epitope conjugated to a terminus of the self-assembling peptide, wherein the anaphylatoxin epitope comprises a C3a anaphylatoxin or a C5a anaphylatoxin, or a combination thereof.

In some embodiments, the anaphylatoxin epitope comprises a C3a anaphylatoxin. In some embodiments, the C3a anaphylatoxin epitope comprises C3a1, C3a2, or C3a3, or a combination thereof. In some embodiments, the anaphylatoxin epitope comprises a C5a anaphylatoxin. In some embodiments, the C5a anaphylatoxin epitope comprises C5a1 or C5a3, or a combination thereof. In some embodiments, the anaphylatoxin epitope comprises C3a1, C3a2, C3a3, C5a1, or C5a3, or a combination thereof. In some embodiments, the anaphylatoxin epitope comprises the amino acid sequence of SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, or SEQ ID NO: 114, or a combination thereof. In some embodiments, each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn). In some embodiments, each self-assembling peptide forms an alpha-helix and comprises a polypeptide having an amino acid sequence of bXXXb (SEQ ID NO: 1), wherein X is independently any amino acid and b is independently any positively charged amino acid. In some embodiments, the self-assembling peptide comprises the sequence QQKFQFQFEQQ (SEQ ID NO: 12) or Ac-QQKFQFQFEQQ-NH₂ (SEQ ID NO: 13) or bXXXb (SEQ ID NO: 1, wherein X is independently any amino acid and b is independently any positively charged amino acid). In some embodiments, b is independently selected from Arg and Lys. In some embodiments, bXXXb (SEQ ID NO: 1) is RAYAR (SEQ ID NO: 2) or KAYAK (SEQ ID NO: 3). In some embodiments, the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20. In some embodiments, the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 9), or Ac-QAKILEADAEILKAYAKILEAHAEILKAQ-NH₂ (SEQ ID NO: 10), or Ac-ADAEILRAYARILEAHAEILRAQ-NH₂ (SEQ ID NO: 11), or QAEILRAYARILEAQ (SEQ ID NO: 101), or QAEILRAYARILEAHAEILKAQ (SEQ ID NO: 102), or QAEILRAYARILEADAKILEAHAEILKAQ (SEQ ID NO: 103), or QAEILRAYARILEADAEILKAQAKILEAHAEILKAQ (SEQ ID NO: 104). In some embodiments, the self-assembling peptide forms a beta sheet and the at least one anaphylatoxin epitope is attached to the C-terminus or the N-terminus of the self-assembling peptide or a combination thereof, or the self-assembling peptide forms an alpha-helix and the at least one anaphylatoxin epitope is attached to the N-terminus of the self-assembling peptide. In some embodiments, 1 to 10 anaphylatoxin epitopes are attached to the C-terminus or the N-terminus of the self-assembling peptide. In some embodiments, the self-assembling peptide forms a beta sheet and the anaphylatoxin conjugate peptide further comprises: (iii) a PEG molecule or a PAS peptide conjugated to the self-assembling peptide. In some embodiments, the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto. In some embodiments, the PEG molecule comprises PEG-2000. In some embodiments, the PEG molecule or the PAS peptide is conjugated to the self-assembling peptide at the same or the opposite terminus from where the anaphylatoxin epitope is attached. In some embodiments, the anaphylatoxin conjugate peptide further includes (iv) at least one linker. In some embodiments, the at least one linker is between the at least one anaphylatoxin epitope and the self-assembling peptide. In some embodiments, the at least one linker comprises a first linker between the at least one anaphylatoxin epitope and the self-assembling peptide, and a second linker between the PEG molecule or the PAS peptide and the self-assembling peptide. In some embodiments, the at least one linker comprises SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) 8), SEQ ID NO: 92 ((G4S) 3), SEQ ID NO: 93 (KSGSG), SEQ ID NO: 94 (KKSGSG), SEQ ID NO: 95 (EAAAK) 2, or SEQ ID NO: 96 (GGAAY). In some embodiments, the anaphylatoxin conjugate peptide comprises C3a1-Coil29, C3a2-Coil29, C3a3-Coil29, C5a1-Coil29, or C5a3-Coil29. In some embodiments, the anaphylatoxin conjugate peptide comprises SEQ ID NO: 115, SEQ ID NO: 116, SEQ ID NO: 117, SEQ ID NO: 118, or SEQ ID NO: 119, or a combination thereof.

In another aspect, provided herein is a nanofiber comprising a plurality of the anaphylatoxin conjugate peptide as detailed herein, wherein the anaphylatoxin conjugate peptides self-assemble into the nanofiber. In another aspect, provided herein is nanofiber comprising: (i) at least one anaphylatoxin conjugate peptide as detailed herein; and (ii) at least one T-cell epitope-conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and at least one T-cell epitope conjugated to a terminus of the self-assembling peptide, wherein the at least one T-cell epitope is selected from PADRE and VAC, and wherein PADRE comprises a polypeptide having the amino acid sequence of aKXVAAWTLKAa (SEQ ID NO: 99, wherein “X” comprises cyclohexylalanine and “a” comprises D-alanine), and wherein VAC comprises a polypeptide having the amino acid sequence of QLVFNSISARALKAY (SEQ ID NO: 100). In some embodiments, the cyclohexylalanine comprises D-alanine. In some embodiments, the T-cell epitope-conjugate peptide further comprises a linker between the T-cell epitope and the self-assembling peptide. In some embodiments, the linker comprises an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) g), SEQ ID NO: 92 ((G4S) 3), SEQ ID NO: 93 (KSGSG), SEQ ID NO: 94 (KKSGSG), SEQ ID NO: 95 (EAAAK) 2, and SEQ ID NO: 96 (GGAAY). In some embodiments, the nanofiber further includes (iii-a) at least one PEG conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), and at least one PEG molecule conjugated to a terminus of the self-assembling peptide; or (iii-b) at least one PAS conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), and at least one PAS peptide conjugated to a terminus of the self-assembling peptide, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto. In some embodiments, the nanofiber further includes (iv) a plain self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn). In some embodiments, the nanofiber comprises a combination of 10 to 10,000, or 100 to 10,000 anaphylatoxin conjugate peptides. In some embodiments, the nanofiber comprises a plurality of the same or different anaphylatoxin epitopes. In some embodiments, the nanofiber comprises a combination of 10 to 10,000, or 100 to 10,000 peptides comprising anaphylatoxin conjugate peptides, T-cell epitope-conjugate peptides, and plain self-assembling peptides. In some embodiments, the nanofiber comprises a combination of 10 to 10,000, or 100 to 10,000 peptides comprising anaphylatoxin conjugate peptides, T-cell epitope-conjugate peptides, PAS conjugate peptides or PEG conjugate peptides, and plain self-assembling peptides. In some embodiments, the nanofiber comprises a combination of 10 to 10,000, or 100 to 10,000 peptides comprising anaphylatoxin conjugate peptides, PAS conjugate peptides or PEG conjugate peptides, and plain self-assembling peptides. In some embodiments, at least about 1%, 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or 97.5% of the peptides in the nanofiber are anaphylatoxin conjugate peptides. In some embodiments, at least about 1%, 2%, 3%, 4%, 5%, 6%, 7%, 8%, 9%, 10%, 11%, 12%, 13%, 14%, or 15% of the peptides in the nanofiber are T-cell epitope-conjugate peptides. In some embodiments, the anaphylatoxin conjugate peptide and the T-cell epitope-conjugate peptide are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1. In some embodiments, the self-assembling peptide forms a fibril including beta-sheet structures or a fibril having a coiled coil structure. In some embodiments, the self-assembling peptide forms a fibril having a structure of a helical filament formed around a central axis. In some embodiments, the N-terminus of each self-assembling peptide is positioned at the exterior of the helical filament. In some embodiments, the anaphylatoxin epitopes are exposed on the exterior surface of the nanofiber. In some embodiments, the nanofiber is about 5-30 nm in width. In some embodiments, the nanofiber is about 100 nm to 1 μm, 100 nm to 2 μm, 100 nm to 3 μm, 100 nm to 4 μm, or 100 nm to 5 μm in length.

In another aspect, provided herein is a pharmaceutical composition comprising: (a) an anaphylatoxin conjugate peptide as detailed herein or a nanofiber as detailed herein; and (b) a pharmaceutically acceptable carrier, diluent, and/or excipient. In some embodiments, the pharmaceutical composition further includes (c) an adjuvant selected from cyclic-di-AMP, CpG, cyclic GMP-AMP (cGAMP), cholera toxin B subunit (CTB), retinoic acid, heat labile toxin B subunit, alum, MF59, 3M-052, iscomatrix, squalene-based adjuvants, AS01, AS03, or AS04, or a combination thereof. In some embodiments, the pharmaceutical composition is formulated into a tablet. In some embodiments, the tablet is a dissolving tablet.

In another aspect, provided herein is a method of treating an inflammatory condition. The method may include administering to a subject a therapeutically effective amount of an anaphylatoxin conjugate peptide as detailed herein, or a nanofiber as detailed herein, or a pharmaceutical composition as detailed herein. In some embodiments, the inflammatory condition comprises inflammatory bowel disease (IBD), sepsis, ulcerative colitis, Crohn's disease, rheumatoid arthritis, psoriasis, cirrhosis, atherosclerosis, or cardiovascular inflammation, lupus, multiple sclerosis, Alzheimer's disease, pulmonary fibrosis, immunosenescence, or a combination thereof. In another aspect, provided herein is a method of reducing inflammation in a subject. The method may include administering to the subject a therapeutically effective amount of an anaphylatoxin conjugate peptide as detailed herein, or a nanofiber as detailed herein, or a pharmaceutical composition as detailed herein. In some embodiments, the inflammation is chronic or acute.

In another aspect, provided herein is a method of treating sepsis. The method may include administering to a subject a therapeutically effective amount of an anaphylatoxin conjugate peptide as detailed herein, or a nanofiber as detailed herein, or a pharmaceutical composition as detailed herein.

In some embodiments, the anaphylatoxin conjugate peptide or the nanofiber or the pharmaceutical composition is administered sublingually. In some embodiments, the subject forms an antibody to the anaphylatoxin epitope. In some embodiments, the antibody comprises IgG and/or IgA.

The disclosure provides for other aspects and embodiments that will be apparent in light of the following detailed description and accompanying figures.

### BRIEF DESCRIPTION OF THE DRAWINGS

FIGS. 1A-1H are images showing radiant efficiency in brains of mice. FIG. 1A are images showing a FITC-CAMP signal in mouse brains over time. The panels show radiant efficiency at 0, 1, 3, 6, 12, and 24 hours after intranasal administration of FITC-labeled cAMP with unlabeled Coil29 nanofibers. FIG. 1B are images showing a TAMRA-Coil29 signal in mouse brains over time. The panels show radiant efficiency 0, 1, 3, 6, 12, and 24 hours after intranasal administration of TAMRA-labeled Coil29 with unlabeled CAMP.

FIG. 1C are images showing a FITC-CAMP signal in lungs and cervical lymph nodes of mice over time. The panels show radiant efficiency at 0, 1, 3, 6, 12, and 24 hours after intranasal administration of FITC-labeled CAMP with unlabeled Coil29 nanofibers. FIG. 1D are images showing a TAMRA-Coil29 signal in lungs and cervical lymph nodes of mice over time. The panels show radiant efficiency 0, 1, 3, 6, 12, and 24 hours after intranasal administration of TAMRA-labeled Coil29 with unlabeled cAMP. FIG. 1E are images showing a FITC-CAMP signal in NALT of mice over time. The panels show radiant efficiency at 0, 1, 3, 6, 12, and 24 hours after intranasal administration of FITC-labeled CAMP with unlabeled Coil29 nanofibers. FIG. 1F are images showing a TAMRA-Coil29 signal in NALT of mice over time. The panels show radiant efficiency 0, 1, 3, 6, 12, and 24 hours after intranasal administration of TAMRA-labeled Coil29 with unlabeled CAMP. FIG. 1G are images showing a FITC-CAMP signal in liver, spleen, heart and kidneys of mice over time. The panels show radiant efficiency at 0, 1, 3, 6, 12, and 24 hours after intranasal administration of FITC-labeled CAMP with unlabeled Coil29 nanofibers. FIG. 1H are images showing a TAMRA-Coil29 signal in liver, spleen, heart and kidneys of mice over time. The panels show radiant efficiency 0, 1, 3, 6, 12, and 24 hours after intranasal administration of TAMRA-labeled Coil29 with unlabeled CAMP.

FIGS. 2A-2H are images and graphs showing that intranasal administration of TAMRA-Coil29+FITC-CAMP results in rapid co-delivery to NALT and lungs. FIG. 2A is an image showing a FITC-cAMP signal in NALT of mice immediately after intranasal administration of FITC-CAMP to the mice. FIG. 2B is a graph showing that one hour following administration, the adjuvant is cleared from NALT of the mice. FIG. 2C is an image showing a TAMRA-Coil29 signal in NALT of mice immediately after intranasal administration of the TAMRA-Coil29 to the mice. FIG. 2D is a graph showing that one hour after administration, the nanofibers are cleared from NALT of the mice. FIG. 2E is an image of a FITC-CAMP signal in lungs of mice immediately after intranasal administration of the FITC-CAMP to the mice. FIG. 2F is a graph showing that three hours after administration, the adjuvant is cleared from the lungs of the mice. FIG. 2G is an image showing a TAMRA-Coil29 signal in lungs of mice immediately after administration of the TAMRA-Coil29 to the mice. FIG. 2H is a graph showing that 24 hours after administration, the nanofibers are cleared from the lungs of the mice.

FIGS. 3A-3D are graphs showing summaries of fluorescent signals over time. FIG. 3A are graphs showing the percent radiant efficiency of a FITC-CAMP signal in cervical lymph nodes and brains of mice at 0, 1, 3, 6, 12, and 24 hours after intranasal administration of FITC-labeled CAMP with unlabeled Coil29 nanofibers. FIG. 3B are graphs showing the percent radiant efficiency of a FITC-CAMP signal in livers, spleens, hearts, and kidneys of mice at 0, 1, 3, 6, 12, and 24 hours after intranasal administration of FITC-labeled CAMP with unlabeled Coil29 nanofibers. FIG. 3C are graphs showing the percent radiant efficiency of a TAMRA-Coil29 signal in cervical lymph nodes and brains of mice at 0, 1, 3, 6, 12, and 24 hours after intranasal administration of TAMRA-labeled Coil29 with unlabeled CAMP. FIG. 3D are graphs showing the percent radiant efficiency of a TAMRA-Coil29 signal in livers, spleens, hearts, and kidneys of mice at 0, 1, 3, 6, 12, and 24 hours after intranasal administration of TAMRA-labeled Coil29 with unlabeled CAMP. For FIGS. 3A-3D, N=3 for each group and mean±SEM is shown. Statistical significance was tested by one-way ANOVA with Tukey's multiple comparison test (FIG. 3A and FIG. 3C), or two-way ANOVA with Tukey's multiple comparison test (FIG. 3B and FIG. 3D). *p<0.05, **p<0.01.

FIGS. 4A-4M are graphs showing LPS challenge outcomes based on the formulation that was administered to mice. FIG. 4A is a plot showing the change summary of survival of LPS challenge outcomes. FIG. 4B is a plot showing the maximum temperature change summary of LPS challenge outcomes. FIG. 4C, FIG. 4D, FIG. 4E, FIG. 4F, FIG. 4G, FIG. 4H, FIG. 4I, FIG. 4J, FIG. 4K, FIG. 4L, and FIG. 4M are graphs showing temperature change over time for groups receiving varying nanofiber formulations, where the title of each graph indicates the concentrations of C3a1-Coil29 and C5a1-Coil29 in the peptide assemblies.

FIGS. 5A-5H show that C5a1-Coil29 raises systemic and mucosal antibody responses when administered intranasally to mice. FIG. 5A is a diagram showing predicted epitopes highlighted on the crystal structure of C5a. FIG. 5B is an atomic force micrograph of C5a1-Coil29 nanofibers and FIG. 5C is an atomic force micrograph of C5a3-Coil29 nanofibers. FIG. 5D is a graph showing IgG titers over time. Mice were immunized at weeks 0, 2, 4, and 7 (dashed vertical lines), and ELISA was performed against the corresponding peptide epitope. FIG. 5E is a graph showing IgG titer at week 5. ELISA was performed against the entire C5a protein. FIG. 5F is a graph showing serum IgA against the C5a peptide that was measured via ELISA. FIG. 5G is a graph showing fecal IgA against the C5a peptide that was measured via ELISA. FIG. 5H is a graph showing fecal IgG against the C5a peptide that was measured via ELISA. N=4-5 for each group. Mean±SEM is shown. Statistical significance was tested by one-way ANOVA with Tukey's multiple comparison test (FIGS. 5E-5H), or two-way ANOVA with Tukey's multiple comparison test (FIG. 5D). *p<0.05, **p<0.01, ***p<0.001, ****p<0.0001.

FIGS. 6A-6K show that C3a1-Coil29 raises systemic antibody responses when administered intranasally to mice and is not a T-cell epitope. FIG. 6A is a diagram showing predicted epitopes highlighted on the crystal structure of C3a. FIG. 6B is an atomic force micrograph of C3a1-Coil29 nanofibers. FIG. 6C is an atomic force micrograph of C3a2-Coil29 nanofibers. FIG. 6D is an atomic force micrograph of C3a3-Coil29 nanofibers. FIG. 6E is a graph showing IgG titers over time. Mice were immunized at weeks 0, 2, 4, and 10 (dashed vertical lines), and ELISA was performed against the corresponding peptide epitope. FIG. 6F is a graph showing IgG titer at week 5. ELISA was performed against the entire C3a protein. FIG. 6G is a graph showing serum IgA against the C5a peptide were measured via ELISA. FIG. 6H is a graph showing fecal IgA against the C5a peptide were measured via ELISA. FIG. 6I is a graph showing fecal IgG against the C5a peptide were measured via ELISA. FIG. 6J is a graph showing that Coil29-stimulated splenocytes reveal bias of Th response in ELISpot. FIG. 6K is a graph showing that epitope-stimulated splenocytes reveal only C3a2 is a T-cell epitope. N=4-5 for each group. Mean±SEM is shown. Statistical significance was tested by one-way ANOVA with Tukey's multiple comparison test (FIGS. 6F-6G), or two-way ANOVA with Tukey's multiple comparison test (FIGS. 6E and FIGS. 6J-6K). *p<0.05, **p<0.01.

FIGS. 7A-7E show that a DoE approach maximizes anti-C3a and anti-C5a titers in a single nanofiber formulation. FIG. 7A is a graph showing DoE design of nanofiber formulations containing C3a1-Coil29 and C5a1-Coil29. FIG. 7B is a surface response plot showing anti-C3a IgG titers that were analyzed via ELISA at week 5 and after three nanofiber administrations. FIG. 7C is a surface response plot showing anti-C5a IgG titers that were analyzed via ELISA at week 5 and after three nanofiber administrations. FIG. 7D is a graph showing epitope IgG titers after a leading formulation containing 1.45 mM C3a1-Coil29 and 0.55 mM C5a1-Coil29, as well as individual epitope formulations, was administered intranasally to mice. The IgG titers were analyzed over time. Dashed line represents boosting schedule. FIG. 7E is a graph showing a comparison of experimental titers to the predicted titers by the DoE at week 5. N=3-10 for each group according to DoE parameters. Mean±SEM is shown.

FIGS. 8A-8O show a correlation between anti-C3a and anti-C5a titers and disease outcomes with LPS challenge following DoE. FIG. 8A is a diagram showing an immunization schedule. Mice were immunized according to the schedule shown, and at the end of the experiment received an LPS challenge. FIG. 8B is a table showing that mice were grouped according to their anti-C3a and anti-C5a IgG titer. FIG. 8C is a graph showing probability of survival of mice over time that were administered varying titers of anti-C3a and anti-C5a IgG or were unimmunized. FIG. 8D is a graph showing temperature change over time for mice with varying titers of anti-C3a and anti-C5a IgG. The first word (low) indicates anti-C3a levels, and second word (low) indicates anti-C5a levels. FIG. 8E is a graph showing temperature change over time for mice with varying titers of anti-C3a and anti-C5a IgG. The first word (low) indicates anti-C3a levels, and second word (medium) indicates anti-C5a levels. FIG. 8F is a graph showing temperature change over time for mice with varying titers of anti-C3a and anti-C5a IgG. The first word (low) indicates anti-C3a levels, and second word (high) indicates anti-C5a levels. FIG. 8G is a graph showing temperature change over time for mice with varying titers of anti-C3a and anti-C5a IgG. The first word (medium) indicates anti-C3a levels, and second word (low) indicates anti-C5a levels. FIG. 8H is a graph showing temperature change over time for mice with varying titers of anti-C3a and anti-C5a IgG. The first word (medium) indicates anti-C3a levels, and second word (medium) indicates anti-C5a levels. FIG. 8I is a graph showing temperature change over time for mice with varying titers of anti-C3a and anti-C5a IgG. The first word (medium) indicates anti-C3a levels, and second word (high) indicates anti-C5a levels. FIG. 8J is a graph showing temperature change over time for mice with varying titers of anti-C3a and anti-C5a IgG. The first word (high) indicates anti-C3a levels, and second word (high) indicates anti-C5a levels. FIG. 8K is a graph showing temperature change over time for mice that were unimmunized and given LPS. FIG. 8L is a graph showing temperature change over time for mice that were unimmunized and given PBS. FIG. 8M is a graph showing the correlation between anti-C3a/anti-C5a titer ratio and percent survival. FIG. 8N is a summary plot of percent survival following LPS challenge. FIG. 8O is a summary plot of maximum temperature change surface response following LPS challenge. N=3-10 for each group according to DoE parameters. Individual values for each mouse are shown. Simple linear regression was used to understand correlation between anti-C3a/anti-C5a ratio and calculate R square value.

FIGS. 9A-9G show that validation and characterization of dual-epitope immunotherapy reveals a Th1-biased immune response and immunity at mucosal tissues. FIG. 9A is a graph showing the number of spots per 250,000 cells in ELISpot against IL-4 and IFNγ following stimulation with Coil29 peptide. FIG. 9B is a graph showing anti-C3a1 IgG subclasses titers that were analyzed via ELISA. FIG. 9C is a graph showing anti-C5a1 IgG subclasses titers that were analyzed via ELISA. FIG. 9D is a graph showing anti-peptide NALT IgG that was analyzed via ELISA at the endpoint of the experiment. FIG. 9E is a graph showing anti-peptide NALT IgA that was analyzed via ELISA at the endpoint of the experiment. FIG. 9F is a graph showing anti-peptide Peyer's patches IgG that was analyzed via ELISA at the endpoint of the experiment. FIG. 9G is a graph showing anti-peptide Peyer's patches IgA that was analyzed via ELISA at the endpoint of the experiment. N=5 for each group. Mean±SEM is shown. Statistical significance was tested by two-way ANOVA with Tukey's multiple comparison test. **p<0.01, ***p<0.001, ****p<0.0001.

### DETAILED DESCRIPTION

Provided herein are novel peptide conjugates that self-assemble into peptide nanofibers or fibrils, with an anaphylatoxin epitope conjugated thereto. These peptide conjugates may be used to prevent and/or treat inflammation or sepsis. The compositions and methods detailed herein may be used as active immunotherapies, which can stimulate a subject's own immune system to raise long lasting, polyclonal adaptive immune responses against problematic self-molecules. Active immunotherapies offer advantages to monoclonal antibodies for treating inflammation because they generate long lasting polyclonal antibody responses. Monoclonal antibodies have other disadvantages, including the need for multiple injections and high rates of primary and secondary failure.

As detailed herein, the inventors used a self-assembling peptide platform to develop an intranasal active immunotherapy against C3a and C5a and studied the effect of targeting each anaphylatoxin immunologically in a model of acute inflammation. Immune responses were generated against these short epitope sequences, which may be advantageous in that only B-cell epitope sequences are made immunogenic, which avoids the generation of autoreactive T-cells. After intranasal immunization, co-delivery of nanofibers and adjuvant to the nasal-associated lymphoid tissue (NALT) and lungs was observed. Utilizing immunogenic epitopes of C5a as well as newly predicted C3a epitopes, systemic and mucosal immunity against each molecule was raised. A design of experiments approach was used to predict maximum antibody responses to each leading epitope in a single immunotherapy platform. In a lipopolysaccharide-induced model of sepsis, anti-C5a responses were protective, increasing survival and decreasing drops in temperature, while anti-C3a responses were detrimental to the model, and survival rates were negatively correlated with anti-C3a/anti-C5a IgG titer ratio. In addition to antibody responses, differences in T-cell profile between C5a, C3a, and C5a+C3a immunized animals were observed. The compositions and methods detailed herein demonstrated functional immunological responses against both complement anaphylatoxins, and shined light on the interplay between the two molecules using a biomaterial-based active immunotherapy platform.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The term “about” or “approximately” as used herein as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, “about” can mean within 3 or more than 3 standard deviations, per the practice in the art. Alternatively, such as with respect to biological systems or processes, the term “about” can mean within an order of magnitude, preferably within 5-fold, and more preferably within 2-fold, of a value.

The term “adjuvant” refers to a compound or mixture that enhances the immune response to an antigen. Adjuvants may contain a substance to protect the antigen from rapid catabolismsuch as aluminum hydroxide or a mineral oil, and also a protein derived from lipid A, Bortadella pertussis, or Mycobacterium tuberculosis. Suitable adjuvants may be commercially available and include, for example, complete or incomplete Freund's adjuvant; AS-2; aluminum salts such as aluminum hydroxide (as a gel, where appropriate) or aluminum phosphate; alum; MF59; calcium salts, iron salts, or zinc salts; an insoluble suspension of acylated tyrosine; acylated sugars; cationically or anionically derivatized polysaccharides; polyphosphazenes; biologically degradable microspheres; monophosphoryl lipid A, cytokines such as GM-CSF, Interleukin-2, Interleukin-7, Interleukin-12, CpG, cholera toxin B subunit (CTB), retinoic acid, heat labile toxin B subunit, 3M-052, iscomatrix, squalene-based adjuvants, AS01, AS03, AS04, STING agonists like cyclic dinucleotides, cyclic-di-AMP, CpG, cyclic GMP-AMP (cGAMP), or a combination thereof.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “antigen” is a molecule capable of being bound by an antibody or T-cell receptor. The term “antigen”, as used herein, also encompasses T-cell epitopes. An antigen also refers to a molecule against which a subject can initiate a humoral and/or cellular immune response leading to the activation of B-lymphocytes and/or T-lymphocytes. An antigen is capable of inducing a humoral immune response and/or cellular immune response leading to the production of B-and/or T-lymphocytes. The structural aspect of an antigen that gives rise to a biological response is referred to herein as an “antigenic determinant.” B-lymphocytes respond to foreign antigenic determinants via antibody production, whereas T-lymphocytes are the mediator of cellular immunity. Thus, antigenic determinants or epitopes are those parts of an antigen that are recognized by antibodies, or in the context of an MHC, by T-cell receptors. An antigenic determinant need not be a contiguous sequence or segment of protein and may include various sequences that are not immediately adjacent to one another. In some embodiments, the antigen contains or is linked to a Th cell epitope. An antigen can have one or more epitopes (B-epitopes and T-epitopes). Antigens may also be mixtures of several individual antigens. Antigens can be any type of biologic molecule including, for example, simple intermediary metabolites, sugars, lipids, and hormones as well as macromolecules such as complex carbohydrates, phospholipids, nucleic acids and proteins. Common categories of antigens include, but are not limited to, viral antigens, bacterial antigens, fungal antigens, protozoa and other parasitic antigens, tumor antigens, antigens involved in autoimmune disease, allergy and graft rejection, and other miscellaneous antigens. Antigens can be microbial antigens, such as viral, fungal, or bacterial; or therapeutic antigens such as antigens associated with cancerous cells or growths, or autoimmune disorders. In some embodiments, the antigen is selected from a small molecule, nucleotide, polynucleotide, peptide, polypeptide, protein, lipid, carbohydrate, other immunogenic molecules, and a combination thereof.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, e.g., to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (e.g., from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be a subject or cell without a composition as detailed herein. A control may be a subject, or a sample therefrom, whose disease state or infection is known. The subject, or sample therefrom, may be healthy, diseased or infected, diseased or infected prior to treatment, diseased or infected during treatment, or diseased or infected after treatment, or a combination thereof.

“Identical” or “identity” as a percentage as used herein in the context of two or more polynucleotide or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

“Immunogenicity” refers to the ability of an antigen to induce an immune response and includes the intrinsic ability of an antigen to generate antibodies in a subject. In some embodiments, the self-assembling peptides described herein, or the nanofibers they form, are not immunogenic without an antigen appended thereto.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Thus, a polynucleotide also encompasses substantially identical polynucleotides and complements thereof. A single strand provides a probe that may hybridize to a target sequence under stringent hybridization conditions. Thus, a polynucleotide also encompasses a probe that hybridizes under stringent hybridization conditions. Polynucleotides may be single stranded or double stranded or may contain portions of both double stranded and single stranded sequence. The polynucleotide can be nucleic acid, natural or synthetic, DNA, genomic DNA, cDNA, RNA, mRNA, or a hybrid, where the polynucleotide can contain combinations of deoxyribo- and ribo-nucleotides, and combinations of bases including, for example, uracil, adenine, thymine, cytosine, guanine, inosine, xanthine hypoxanthine, isocytosine, and isoguanine. Polynucleotides can be obtained by chemical synthesis methods or by recombinant methods.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The terms “polypeptide”, “protein,” and “peptide” are used interchangeably herein. “Primary structure” refers to the amino acid sequence of a particular peptide. “Secondary structure” refers to locally ordered, three dimensional structures within a polypeptide. Secondary structure may include beta-sheet and alpha-helices. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three dimensional structure of a polypeptide monomer. “Quaternary structure” refers to the three dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. In some embodiments, a carrier includes a solution at neutral pH. In some embodiments, a carrier includes a salt. In some embodiments, a carrier includes a buffered solution. In some embodiments, a carrier includes phosphate buffered saline solution.

“Sample” or “test sample” as used herein can mean any sample in which the presence and/or level of a target is to be detected or determined or a portion from a subject or portion of an immunogenic composition as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” and “patient” as used herein interchangeably refers to any vertebrate, including, but not limited to, a mammal that wants or is in need of the herein described compositions or methods. The subject may be a human or a non-human. The subject may be a vertebrate. The subject may be a mammal. The mammal may be a primate or a non-primate. The mammal can be a non-primate such as, for example, cow, pig, camel, llama, hedgehog, anteater, platypus, elephant, alpaca, horse, goat, rabbit, sheep, hamster, guinea pig, cat, dog, rat, and mouse. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12 years, or an infant, such as age 0-1 years. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99%, or less than 100% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Treatment” or “treating” or “therapy” when referring to protection of a subject from a disease, means suppressing, repressing, reversing, alleviating, ameliorating, or inhibiting the progress of disease, or completely eliminating a disease. A treatment may be either performed in an acute or chronic way. The term also refers to reducing the severity of a disease or symptoms associated with such disease prior to affliction with the disease. Treatment may result in a reduction in the incidence, frequency, severity, and/or duration of symptoms of the disease. Preventing the disease involves administering a composition of the present invention to a subject prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. A disease may include a bacterial infection. Bacterial infections may occur when bacteria enter the body of a subject, increase in number, and cause a reaction in the body. Bacteria can enter the body through an opening in the skin of a subject, such as a cut or surgical wound, or through orifices in the body of a subject, such as the airway (for example, nasal passages, mouth), urethra, ear canal, eye, and the like. A bacterial infection can be caused by either gram-negative or gram-positive bacteria. In some embodiments, the disease includes inflammation. In some embodiments, the disease includes sepsis.

“Variant” used herein with respect to a polynucleotide means (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequence substantially identical thereto. A variant can be a polynucleotide sequence that is substantially identical over the full length of the full polynucleotide sequence or a fragment thereof. The polynucleotide sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the polynucleotide sequence or a fragment thereof.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. Representative examples of “biological activity” include the ability to be bound by a specific antibody or polypeptide or to promote an immune response. Variant can mean a functional fragment thereof. Variant can also mean multiple copies of a polypeptide. The multiple copies can be in tandem or separated by a linker. A conservative substitution of an amino acid, for example, replacing an amino acid with a different amino acid of similar properties (for example, hydrophilicity, degree and distribution of charged regions) is recognized in the art as typically involving a minor change. These minor changes may be identified, in part, by considering the hydropathic index of amino acids, as understood in the art (Kyte et al., J. Mol. Biol. 1982, 157, 105-132, incorporated herein by reference). The hydropathic index of an amino acid is based on a consideration of its hydrophobicity and charge. It is known in the art that amino acids of similar hydropathic indexes may be substituted and still retain protein function. In one aspect, amino acids having hydropathic indexes of +2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within +2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. A variant can be an amino acid sequence that is substantially identical over the full length of the amino acid sequence or fragment thereof. The amino acid sequence can be 80%, 81%, 82%, 83%, 84%, 85%, 86%, 87%, 88%, 89%, 90%, 91%, 92%, 93%, 94%, 95%, 96%, 97%, 98%, or 99%, or less than 100% identical over the full length of the amino acid sequence or a fragment thereof.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear; in the event however of any latent ambiguity, definitions provided herein take precedent over any dictionary or extrinsic definition. Further, unless otherwise required by context, singular terms shall include pluralities and plural terms shall include the singular.

### 2. Anaphylatoxin Conjugate Peptide

Provided herein is an anaphylatoxin conjugate peptide. The anaphylatoxin conjugate peptide may include a self-assembling peptide and at least one anaphylatoxin epitope attached thereto.

a. Self-Assembling Peptide

The compositions and methods detailed herein include self-assembling peptides. As used herein, the term “self-assembling peptide” refers to peptides that are able to spontaneously associate and form stable structures. Each self-assembling peptide may comprise or form an alpha helix. In other embodiments, each self-assembling peptide may comprise or form a beta-sheet. Examples of self-assembling peptides are detailed in, for example, U.S. Pat. Nos. 9,241,987; 9,849,174; 10,596,238; 11,246,924; International Patent Application Publication No. WO 2023/044163; Lee, S. et al. Int. J. Mol. Sci. 2019, 20, 5850; Hernandez, A. et al. Front. Bioeng. Biotechnol. 2023, 11, 1139782; and Lopez-Silva et al. ACS Biomater. Sci. Eng. 2019, 5, 977-985, each of which is incorporated herein by reference in its entirety.

In some embodiments, the self-assembling peptide comprises a polypeptide having an amino acid sequence selected from bXXXb (SEQ ID NO: 1, wherein X is independently any amino acid and b is independently any positively charged amino acid), QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), or a polypeptide with at least 75%, 80%, 85%, or 90% identity thereto. Some examples of self-assembling peptide are detailed in, for example, U.S. Pat. Nos. 9,241,987; 9,849,174; and 10,596,238, each of which is incorporated herein by reference in its entirety. In some embodiments, the self-assembling peptide comprises the amino acid sequence of QQKFQFQFEQQ (Q11, SEQ ID NO: 12), or a polypeptide with at least 75%, 80%, 85%, or 90% identity thereto. In some embodiments, the self-assembling peptide comprises the sequence Ac-QQKFQFQFEQQ-NH₂ (Q11, SEQ ID NO: 13), or a polypeptide with at least 75%, 80%, 85%, or 90% identity thereto.

The self-assembling peptide may comprise an amino acid sequence of bXXXb (SEQ ID NO: 1), wherein X is independently any amino acid, and b is independently any positively charged amino acid. In such embodiments, each self-assembling peptide may form an alpha helix. In some embodiments, b is independently selected from Arg and Lys. In some embodiments, b is Arg. In some embodiments, b is Lys. In some embodiments, bXXXb (SEQ ID NO: 1) is RAYAR (SEQ ID NO: 2). In some embodiments, bXXXb (SEQ ID NO: 1) is KAYAK (SEQ ID NO: 3). In some embodiments, the self-assembling peptide comprises the sequence of RXXXR (SEQ ID NO: 4), wherein X is any amino acid. The self-assembling peptide may comprise an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20. In some embodiments, n is an integer from 5 to 15, and m is an integer from 5 to 15. Some examples of self-assembling peptide are detailed in, for example, U.S. Pat. No. 11,246,924, which is incorporated herein by reference in its entirety. In such embodiments, a plurality of the anaphylatoxin conjugate peptides may assemble into a nanofiber.

In some embodiments, the self-assembling peptide comprises a glutamine at the C-terminus. In some embodiments, the self-assembling peptide comprises a glutamine at the N-terminus. The self-assembling peptide may include at least, at most, or exactly 5, 10, 15, 16, 17, 18, 19, 20, 21, 22, 23, 24, 25, 26, 27, 28, 29, 30, 31, 32, 33, 34, 35, or 40 amino acids. In some embodiments, the self-assembling peptide comprises 5 to 40 amino acids in length.

In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or Ac-QARILEADAEILRAYARILEAHAEILRAQ-NH₂ (SEQ ID NO: 9) or Ac-QAKILEADAEILKAYAKILEAHAEILKAQ-NH₂ (SEQ ID NO: 10) or Ac-ADAEILRAYARILEAHAEILRAQ-NH₂ (SEQ ID NO: 11), or QAEILRAYARILEAQ (SEQ ID NO: 101), or QAEILRAYARILEAHAEILKAQ (SEQ ID NO: 102), or QAEILRAYARILEADAKILEAHAEILKAQ (SEQ ID NO: 103), or QAEILRAYARILEADAEILKAQAKILEAHAEILKAQ (SEQ ID NO: 104), or a polypeptide with at least 75%, 80%, 85%, 90%, or 95% identity thereto. In some embodiments, the self-assembling peptide comprises an amino acid sequence of QARILEADAEILRAYARILEAHAEILRAQ (SEQ ID NO: 6) or QAKILEADAEILKAYAKILEAHAEILKAQ (SEQ ID NO: 7) or ADAEILRAYARILEAHAEILRAQ (SEQ ID NO: 8) or a variant thereof. In some embodiments, the self-assembling peptide comprises an amino acid sequence of QARILEADAEILRAYARILEAHAEILRAQ (SEQ ID NO: 6). In some embodiments, the self-assembling peptide comprises an amino acid sequence of QAKILEADAEILKAYAKILEAHAEILKAQ (SEQ ID NO: 7). In some embodiments, the self-assembling peptide comprises an amino acid sequence ofADAEILRAYARILEAHAEILRAQ (SEQ ID NO: 8).

In some embodiments, each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), or a polypeptide with at least 75%, 80%, 85%, or 90% identity thereto. In such embodiments, a plurality of the anaphylatoxin conjugate peptides may assemble into a nanofiber.

In some embodiments, the self-assembling peptide comprises a polypeptide having an amino acid sequence selected from VEVKVEVKV (SEQ ID NO: 41), VEVKVEVKVEVK (SEQ ID NO: 42), VVVAAAEEE (SEQ ID NO: 43), VEVEVEVEVEVEVEVEVEVE (SEQ ID NO: 44), CGNKRTRGC (SEQ ID NO: 45), VKVKVKVKVDPPTKVEVKVKV (SEQ ID NO: 46), LRKKLGKA (SEQ ID NO: 47), WVVVVVKK (SEQ ID NO: 48), AEAKAEAKAEAKAEAK (SEQ ID NO: 49), AEAKAEAK (SEQ ID NO: 50), AEAEAEAEAKAK (SEQ ID NO: 51), AEAEAKAK (SEQ ID NO: 52), AEAEAKAKAEAEAKAK (SEQ ID NO: 53), RADARADARADARADA (SEQ ID NO: 54), RADARGDARADARGDA (SEQ ID NO: 55), RADARADA (SEQ ID NO: 56), RARADADARARADADA (SEQ ID NO: 57), RARADADA (SEQ ID NO: 58), RARARARADADADADA (SEQ ID NO: 59), ADADADADARARARAR (SEQ ID NO: 60), DADADADARARARARA (SEQ ID NO: 61), RAEARAEARAEARAEA (SEQ ID NO: 62), RAEARAEA (SEQ ID NO: 63), KAKAKAKAEAEAEAEA (SEQ ID NO: 64), AEAEAEAEAKAKAKAK (SEQ ID NO: 65), KADAKADAKADAKADA (SEQ ID NO: 66), KADAKADA (SEQ ID NO: 67), AEAEAHAHAEAEAHAHA (SEQ ID NO: 68), AEAEAHAHA (SEQ ID NO: 69), HEHEHKHKHEHEHKHK (SEQ ID NO: 70), HEHEHKHK (SEQ ID NO: 71), FEFEFKFKFEFEFKFK (SEQ ID NO: 72), FEFKFEFK (SEQ ID NO: 73), LELELKLKLELELKLK (SEQ ID NO: 74), LELELKLK (SEQ ID NO: 75), KFDLKKDLKLDL (SEQ ID NO: 76), FKFEFKFF (SEQ ID NO: 77), FEFEFKFK (SEQ ID NO: 78), and RFRFRFRFRFRFRFRFRFRF (SEQ ID NO: 79), or a polypeptide with at least 75%, 80%, 85%, or 90% identity thereto. In such embodiments, a plurality of the PC-peptide conjugates may assemble into a nanofiber, nanotube, hydrogel, micelle, vesicle, nanoparticle, or suspension.

In some embodiments, the self-assembling polypeptide includes a modification to the C-terminus, to the N-terminus, or to both the C-terminus and N-terminus. N-terminal modifications may include, for example, biotin and acetyl. C-terminal modifications may include, for example, amide. In some embodiments, the N-terminus is acetylated (which may be indicated by “Ac” for example). In some embodiments, the C-terminus is amidated (which may be indicated by “NH₂” for example).

The peptides described herein can be chemically synthesized using standard chemical synthesis techniques. In some embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides described herein. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc., 85:2149-2156; and Stewart et al. (1984) Solid Phase Peptide Synthesis, 2nd ed. Pierce Chem. Co., Rockford, III; each of which is incorporated herein by reference. In some embodiments, the self-assembling peptide is synthesized by a solid phase peptide synthesis.

b. Anaphylatoxin Epitope

The anaphylatoxin conjugate peptide includes at least one anaphylatoxin epitope. The anaphylatoxin epitope may be a B-cell epitope. The anaphylatoxin epitope may be a T-cell epitope. The anaphylatoxin epitope may comprise a C3a anaphylatoxin or a C5a anaphylatoxin, or a combination thereof. In some embodiments, the anaphylatoxin epitope comprises a C3a anaphylatoxin. C3a anaphylatoxin epitopes may include C3a1, C3a2, or C3a3, or a combination thereof. In some embodiments, the anaphylatoxin epitope comprises a C5a anaphylatoxin. C5a anaphylatoxin epitopes may include C5a1 or C5a3, or a combination thereof. The anaphylatoxin epitope may comprise C5a1 (KESPHKPVQLGR, SEQ ID NO: 105), C5a3 (EERVARVTIGPLCIRA, SEQ ID NO: 106), C3a1 (DKGLRKCCEDG, SEQ ID NO: 107), C3a2 (RYSCQRRAR, SEQ ID NO: 108), or C3a3 (IKAFIDCCNHITK, SEQ ID NO: 109), or a combination thereof. The anaphylatoxin epitope may be selected from C5a1 (KESPHKPVQLGR, SEQ ID NO: 105), C5a3 (EERVARVTIGPLCIRA, SEQ ID NO: 106), C3a1 (DKGLRKCCEDG, SEQ ID NO: 107), C3a2 (RYSCQRRAR, SEQ ID NO: 108), or C3a3 (IKAFIDCCNHITK, SEQ ID NO: 109), or a combination thereof, or a peptide having at least 80%, 81%, 82%, 83%, 84%, 85%, 86%, 87%, 88%, 89%, 90%, 91%, 92%, 93%, 94%, 95%, 96%, 97%, 98%, or 99% identity thereto. In some embodiments, the peptide fibril is coupled to a plurality of anaphylatoxin epitopes. The plurality of anaphylatoxin epitopes coupled to a peptide fibril may be the same or different. Anaphylatoxin epitopes may be obtained commercially or synthesized and purified by any suitable method known in the art. In some embodiments, a peptide detailed herein includes C-terminal amidation. For example, the anaphylatoxin epitope may comprise C5a1 with C-terminal NH₂ (NH₂-KESPHKPVQLGR-NH₂, SEQ ID NO: 110), C5a3 with C-terminal NH₂ (NH₂-EERVARVTIGPLCIRA-NH₂, SEQ ID NO: 111), C3a1 with C-terminal NH₂ (NH₂-DKGLRKCCEDG-NH₂, SEQ ID NO: 112), C3a2 with C-terminal NH₂ (NH2-RYSCQRRAR-NH2, SEQ ID NO: 113), and/or C3a3 with C-terminal NH2 (NH2-IKAFIDCCNHITK-NH2, SEQ ID NO: 114). In some embodiments, a peptide detailed herein includes acetylation.

The anaphylatoxin epitope may be conjugated or coupled to a self-assembling peptide by any means known in the art. The anaphylatoxin epitope may be covalently coupled to a terminus of the self-assembling peptide. At least one anaphylatoxin epitope may be attached to the C-terminus or the N-terminus of the self-assembling peptide. In some embodiments, the anaphylatoxin epitope is covalently coupled to the N-terminus or N-terminal end of the self-assembling peptide. In some embodiments, the anaphylatoxin epitope is covalently coupled to the C-terminus or C-terminal end of the self-assembling peptide. In some embodiments, the self-assembling peptide forms a beta sheet and the at least one anaphylatoxin epitope is attached to the C-terminus or the N-terminus of the self-assembling peptide or a combination thereof. In some embodiments, the self-assembling peptide forms an alpha-helix and the at least one anaphylatoxin epitope is attached to the N-terminus of the self-assembling peptide. The conjugation of the anaphylatoxin epitope to the N-terminus or the N-terminal end of the self-assembling peptide may orient the anaphylatoxin epitope towards the exterior of the helical peptide fibril once a plurality of anaphylatoxin conjugate peptides assembles into a nanofiber. In some embodiments, the anaphylatoxin epitopes are exposed on the exterior surface of the nanofiber. In some embodiments, the anaphylatoxin epitopes are exposed on the exterior surface of the helical filament of the nanofiber.

The anaphylatoxin conjugate peptide may include at least one anaphylatoxin epitope. The anaphylatoxin conjugate peptide may include 1 to 10 anaphylatoxin epitopes attached to a self-assembling peptide. The anaphylatoxin conjugate peptide may include 1, 2, 3, 4, 5, 6, 7, 8, 9, or 10 anaphylatoxin epitopes attached to a self-assembling peptide. In embodiments including more than one anaphylatoxin epitope attached to a self-assembling peptide, all anaphylatoxin epitopes may be attached to the same end of the self-assembling peptide. For example, 1, 2, 3, or 4 anaphylatoxin epitopes together may be attached to the N-terminal end or to the C-terminal end of the self-assembling peptide. Once assembled into a nanofiber, the nanofiber may include n anaphylatoxin epitopes, wherein n is an integer from 1 to 1,000, or 1 to 5,000, or 1 to 10,000, or 1 to 50,000, or 1 to 100,000, including all values and ranges there between. The anaphylatoxin epitopes attached to a self-assembling peptide may be the same anaphylatoxin epitope or different anaphylatoxin epitopes.

In some embodiments, the antigens are exposed on the surface of the peptide fibril. In certain aspects, the ratio of antigen to self-assembling peptide is 1:1000, 1:100:1:10, or 1:1, including all values and ranges there between.

c. Linker

The anaphylatoxin conjugate peptide may further comprise a linker. The linker may be between the anaphylatoxin epitope and the self-assembling peptide. The linker may be between the at least one anaphylatoxin epitope and the self-assembling peptide. In some embodiments, a linker is covalently attached to the self-assembling peptide between the anaphylatoxin epitope and the self-assembling peptide. In some embodiments, the linker comprises at least one cysteine. The at least one cysteine may be at the N-terminus of the linker. In some embodiments, the linker comprises glycine and serine. In some embodiments, the linker comprises glycine and serine and cysteine. In some embodiments, the anaphylatoxin epitope is attached to the self-assembling peptide through a thiol reactive group in the linker.

In some embodiments, the conjugate peptide includes more than one linker. In such embodiments, the linkers may be the same or different from one another. The conjugate peptide may include at least 1, at least 2, at least 3, at least 4, at least 5, at least 6, at least 7, at least 8, at least 9, or at least 10 linkers. The conjugate peptide may include less than 20, less than 15, less than 10, or less than 5 linkers. The conjugate peptide may include between 1 and 20, between 5 and 15, or between 1 and 5 linkers. The linker may be positioned at the C-terminus of the self-assembling peptide, at the N-terminus of the self-assembling peptide, or at both the N- and C-termini of the self-assembling peptide. In some embodiments, the linker is positioned at the N-terminus of the self-assembling peptide. Multiple linkers may be positioned adjacent to one another.

The linker may comprise, for example, an oligoethylene glycol, polyethylene glycol, or an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) 8), SEQ ID NO: 92 ((G4S) 3), SEQ ID NO: 93 (KSGSG), SEQ ID NO: 94 (KKSGSG), SEQ ID NO: 95 (EAAAK) 2, and SEQ ID NO: 96 (GGAAY). In some embodiments, the linker comprises (Ser-Gly)₂ (SEQ ID NO: 85).

d. PEG Molecule or PAS Peptide

The anaphylatoxin conjugate peptide may further comprise a polyethylene glycol (PEG) molecule. The self-assembling polypeptide may include a PEG molecule instead of an anaphylatoxin. A self-assembling polypeptide with a PEG molecule attached thereto may be referred to as a PEG conjugate peptide. Accordingly, provided herein is a PEG conjugate peptide, which may include a self-assembling polypeptide as detailed above with a PEG molecule attached thereto. The PEG molecule may facilitate mucus penetration. The PEG molecule may be muco-inert. The PEG molecule may enable or facilitate oral availability. The PEG molecule may be an average molecular weight from 1000 Da and up to and including 100,000 Da, or from 1000 Da and up to and including 5,000 Da. The PEG molecule may comprise PEG2000, PEG1000, or PEG3000. In some embodiments, the PEG molecule comprises PEG2000. The PEG molecule may comprise CH₃O—(CH₂CH₂°) n wherein n is an integer from about 1 to about 2000, or from about 1 to about 100, or from about 20 to about 70. In some embodiments, the average n is at least about 20, about 45, or less than about 70. A plurality of anaphylatoxin conjugate peptides may have the same or different PEG molecule attached to each self-assembling peptide. The PEG molecule may be conjugated to a beta-sheet self-assembling peptide at the N-terminus or N-terminal end or at the C-terminus or C-terminal end or at both ends. The PEG molecule may be conjugated to a beta-sheet self-assembling peptide at the N-terminus or N-terminal end or at the C-terminus or C-terminal end, and include an anaphylatoxin conjugated at the other of the N-terminus or N-terminal end or the C-terminus or C-terminal end. The PEG molecule may be conjugated to the self-assembling peptide at the opposite terminus from wherein the anaphylatoxin epitope is conjugated. The PEG molecule may be conjugated to an alpha-helical self-assembling peptide at the N-terminus or N-terminal end. The PEG molecule may be conjugated to the self-assembling peptide via a linker, as detailed above. The PEG molecule may be commercially obtained.

The anaphylatoxin conjugate peptide may further comprise a PAS peptide. The self-assembling polypeptide may include a PAS peptide instead of an anaphylatoxin. A self-assembling polypeptide with a PAS peptide attached thereto may be referred to as a PAS conjugate peptide. Accordingly, provided herein is a PAS conjugate peptide, which may include a self-assembling polypeptide as detailed above with a PAS peptide attached thereto. The PAS peptide may be muco-inert. The PAS peptide may facilitate mucus penetration. The PAS peptide may enable or facilitate oral availability. The PAS peptide may comprise the amino acid sequence of Pro-Ala-Ser. The PAS peptide may comprise the amino acid sequence of ASPAAPAPASPAAPAPSAPA (SEQ ID NO: 97), or a peptide having at least 80%, 85%, 90%, or 95% identity thereto. In some embodiments, the PAS peptide comprises ASPAAPAPASPAAPAPSAPA (SEQ ID NO: 97). In some embodiments, the PAS peptide comprises H₂N-ASPAAPAPASPAAPAPSAPA-NH₂ (SEQ ID NO: 98). The PAS peptide may be conjugated to a beta-sheet self-assembling peptide at the N-terminus or N-terminal end or at the C-terminus or C-terminal end or at both ends. The PAS peptide may be conjugated to a beta-sheet self-assembling peptide at the N-terminus or N-terminal end or at the C-terminus or C-terminal end, and include an anaphylatoxin conjugated at the other of the N-terminus or N-terminal end or the C-terminus or C-terminal end. The PAS peptide may be conjugated to the self-assembling peptide at the opposite terminus from wherein the anaphylatoxin epitope is conjugated. The PAS peptide may be conjugated to an alpha-helical self-assembling peptide at the N-terminus or N-terminal end. The PAS peptide may be conjugated to the self-assembling peptide via a linker, as detailed above.

In some embodiments, the anaphylatoxin conjugate peptide does not include a PEG molecule or a PAS peptide. In some such embodiments, the self-assembling peptide and/or the anaphylatoxin epitope may have a hydrophobicity index such that the anaphylatoxin conjugate peptide has sufficient activity or effectiveness without a PEG molecule or a PAS peptide. In some embodiments, a more hydrophobic epitope may need more of the conjugate peptides in a nanofiber to be PASylated or PEGylated to have a desired activity or effectiveness. In some embodiments, a more hydrophilic epitope and/or self-assembling peptide may not need any or may need less of the conjugate peptides in a nanofiber to be PASylated or PEGylated to have a desired activity or effectiveness.

The anaphylatoxin conjugate peptide may comprise, for example, C3a1-Coil29, C3a2-Coil29, C3a3-Coil29, C5a1-Coil29, or C5a3-Coil29, or a combination thereof. Exemplary conjugate peptides are shown in TABLE 1. The anaphylatoxin conjugate peptide may comprise, for example, any one of SEQ ID NOs: 105-119, or a combination thereof. The conjugate peptide may comprise, for example, any one of SEQ ID NOs: 105-121, or a combination thereof.

TABLE 1
Synthetic peptide sequences.

| Peptide | Sequence |
|---|---|
| Ac-Coil29 | Ac-QARILEADAEILRAYARILEAHAEILRAQ-NH2 (SEQ ID NO: 9) |
| PADRE | NH2-aKXVAAWTLKAa-NH2 (SEQ ID NO: 120) |
| | X = cyclohexyl alanine, a = D-alanine |
| PADRE- | NH2-aKXVAAWTLKAa-SGSG-QARILEADAEILRAYARILEAHAEILRAQ- |
| Coil29 | NH2 (SEQ ID NO: 121) |
| | X = cyclohexyl alanine, a = D-alanine |
| C5a1- | NH2-KESPHKPVQLGR-SGSGEAAAKEAAAKSGSG- |
| Coil29 | QARILEADAEILRAYARILEAHAEILRAQ-NH2 (SEQ ID NO: 115) |
| C5a3- | NH2-EERVARVTIGPLCIRA-SGSGEAAAKEAAAKSGSG- |
| Coil29 | QARILEADAEILRAYARILEAHAEILRAQ-NH2 (SEQ ID NO: 116) |
| pC3a1 | NH2-DKGLRKCCEDG-NH2 (SEQ ID NO: 112) |
| pC3a2 | NH2-RYSCQRRAR-NH2 (SEQ ID NO: 113) |
| pC3a3 | NH2-IKAFIDCCNHITK-NH2 (SEQ ID NO: 114) |
| C3a1- | NH2-DKGLRKCCEDG-SGSGEAAAKEAAAKSGSG- |
| Coil29 | QARILEADAEILRAYARILEAHAEILRAQ-NH2 (SEQ ID NO: 117) |
| C3a2- | NH2-RYSCQRRAR-SGSGEAAAKEAAAKSGSG- |
| Coil29 | QARILEADAEILRAYARILEAHAEILRAQ-NH2 (SEQ ID NO: 118) |
| C3a3- | NH2-IKAFIDCCNHITK-SGSGEAAAKEAAAKSGSG- |
| Coil29 | QARILEADAEILRAYARILEAHAEILRAQ-NH2 (SEQ ID NO: 119) |
| TAMRA- | TAMRA-SGSGEAAAKEAAAKSGSG- |
| Coil29 | QARILEADAEILRAYARILEAHAEILRAQ-NH2 (SEQ ID NO: 122) |

*C-terminal NH2 indicates amidation and Ac means acetylation.

### 3. T-cell Epitope Conjugate Peptides

Further provided herein is a T-cell epitope conjugate peptide. The T-cell epitope conjugate peptide may include a self-assembling peptide and at least one T-cell epitope attached thereto. The self-assembling peptide of the T-cell epitope conjugate peptide may be as detailed above for the anaphylatoxin conjugate peptide. The T-cell epitope may be selected from PADRE and VAC. The PADRE molecule may comprise a polypeptide having the amino acid sequence of aKXVAAWTLKAa, wherein “X” comprises cyclohexylalanine and “a” comprises D-alanine (SEQ ID NO: 99). In some embodiments, the cyclohexylalanine comprises D-alanine. The VAC molecule may comprise a polypeptide having the amino acid sequence of QLVFNSISARALKAY (SEQ ID NO: 100).

In some embodiments, the T-cell epitope conjugate peptide further comprises a linker between the T-cell epitope and the self-assembling peptide. The linker of the T-cell epitope conjugate peptide may be as detailed above for the anaphylatoxin conjugate peptide.

### 4. Plain Self-Assembling Peptides

Some embodiments include plain self-assembling peptides, which refers to a self-assembling peptide as detailed above, but without any antigen such as an anaphylatoxin epitope or a T-cell epitope (such as PADRE or VAC) or a PEG molecule or a PAS peptide attached thereto.

### 5. Nanofibers

Further described herein is a platform for vaccination or treatment based on peptides assembled into nanofibers. Provided herein is a nanofiber comprising a plurality of conjugate peptides, including anaphylatoxin conjugate peptides. The anaphylatoxin conjugate peptides, with or without other conjugate peptides such as PEG conjugate peptides or PAS conjugate peptides or T-cell epitope conjugate peptides or plain self-assembling peptides, may self-assemble into a nanofiber. Self-assembly may refer to a process by which a plurality of conjugate peptides come together to form an organized structure such as a nanofiber or micelle or other larger form, based on interactions between conjugate peptides such as covalent, ionic, and Van der Waals interactions. For example, each type of conjugate peptide may be synthesized separately, and then multiple conjugate peptides may be mixed together in various ratios to form or assemble together into integrated nanofibers. The nanofiber may also be referred to as a peptide fibril. The nanofibers may be comprised of alpha-helical peptides. The nanofibers may be comprised of beta-sheet peptides. In this strategy, peptides fold into a complex beta-sheet-based or alpha-helix-based nanofiber where individual peptide coils run perpendicular to the axis of a long fibril. Each self-assembling peptide may comprise or form an alpha helix. The plurality of self-assembling peptides may form a peptide fibril in the form of a helical filament. The resultant nanostructure is composed of thousands of individual peptides or more. The self-assembling peptide may be extended N-terminally with a flexible spacer and an immune epitope such as an anaphylatoxin epitope. In some embodiments, the nanofiber does not further comprise an adjuvant. In some embodiments, the nanofiber is an adjuvant.

Multiple epitope-bearing self-assembling peptides may be co-assembled into nanofibers composed of β-sheets or α-helices. Coiled coil folding requires more extensive design considerations compared to β-sheet fibrillization, as both inter-helical interactions as well as those between the C-terminus and the main chain must be considered.

The nanofiber may be comprised of 10 to 10,000 peptides including all values and ranges there between. The nanofiber may include a combination of 10 to 10,000, or 100 to 10,000 peptides comprising anaphylatoxin conjugate peptides. The nanofiber may comprise a combination of 10 to 10,000, or 100 to 10,000 peptides comprising anaphylatoxin conjugate peptides, T-cell epitope conjugate peptides, and plain self-assembling peptides. The nanofiber may comprise a combination of 10 to 10,000, or 100 to 10,000 peptides comprising anaphylatoxin conjugate peptides, PAS or PEG conjugate peptides, and plain self-assembling peptides. The nanofiber may comprise a combination of 10 to 10,000, or 100 to 10,000 peptides comprising anaphylatoxin conjugate peptides, T-cell epitope conjugate peptides, PAS or PEG conjugate peptides, and plain self-assembling peptides.

The nanofiber may include at least one anaphylatoxin conjugate peptide. The nanofiber may include a plurality of anaphylatoxin conjugate peptides. The nanofiber may include at least 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, or 9000, or less than 10,000 of the same or different anaphylatoxin conjugate peptides. The anaphylatoxin conjugate peptides making up a single nanofiber may be the same or different. The nanofiber may further include at least one T-cell epitope conjugate peptide. The nanofiber may include a plurality of T-cell epitope conjugate peptides. The nanofiber may include at least 1, 5, 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, or 5000 of the same or different T-cell epitope conjugate peptides. The T-cell epitope conjugate peptides making up a single nanofiber may be the same or different. In some embodiments, the nanofiber includes self-assembling peptides without a T-cell epitope or without an anaphylatoxin epitope conjugated thereto, which may be referred to as a plain self-assembling peptide. The nanofiber may further include at least one plain self-assembling peptide. The nanofiber may include a plurality of plain self-assembling peptides. The nanofiber may include at least 10, 20, 30, 40, 50, 60, 70, 80, 90, 100, 150, 200, 250, 300, 400, 450, 500, 550, 600, 650, 700, 750, 800, 850, 900, 950, 1000, 1200, 1400, 1600, 1800, 2000, 2200, 2400, 2600, 2800, 3000, 3200, 3400, 3600, 3800, 4000, 4200, 4400, 4600, 4800, 5000, 5200, 5400, 5600, 5800, 6000, 6200, 6400, 6600, 6800, 7000, 7200, 7400, 7600, 7800, 8000, 8200, 8400, 8600, 8800, or 9000, or less than 10,000 of the same or different plain self-assembling peptides. The plain self-assembling peptides making up a single nanofiber may be the same or different.

A single nanofiber may include different peptides in a variety of ratios. The nanofiber may include a variety of ratios of different anaphylatoxin epitopes. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are C5a1 anaphylatoxin conjugate peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are C5a3 anaphylatoxin conjugate peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are C3a1 anaphylatoxin conjugate peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are C3a2 anaphylatoxin conjugate peptides. In some embodiments, at least about 1%, 5%, 10%, 15%, 20%, 25%, 30%, 35%, 40%, 45%, 50%, 55%, 60%, 65%, 70%, 75%, 80%, 85%, 90%, 95%, or 97.5% of the peptides in the nanofiber are C3a3 anaphylatoxin conjugate peptides. In some embodiments, at least about 1%, 2%, 3%, 4%, 5%, 6%, 7%, 8%, 9%, 10%, 11%, 12%, 13%, 14%, or 15% of the peptides in the nanofiber are T-cell epitope conjugate peptides. In some embodiments, at least about 1%, 10%, 12.5%, 15%, 17.5%, 20%, 22.5%, 25%, 27.5%, 30%, 32.5%, 35%, 37.5%, 40%, 42.5%, 45%, 47.5%, 50%, 52.5%, 55%, 57.5%, 60%, 62.5%, 65%, 67.5%, 70%, 72.5%, 75%, 77.5%, 80%, 82.5%, 85%, 87.5%, 90%, 92.5%, 95%, or 96.5%, or less than about 96.5% of the peptides in the nanofiber are plain self-assembling peptides. In some embodiments, the anaphylatoxin peptide conjugate and the T-cell epitope peptide conjugate are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

The helical filament of the nanofiber may be formed around a central axis or core. The plurality of self-assembling peptides may form a peptide fibril in the form of a coiled coil. In some embodiments, the N-terminus of each self-assembling peptide is positioned at the exterior of the helical filament. The anaphylatoxin epitopes may be exposed on the exterior surface of the nanofiber. An example of the self-assembling peptides formed into a peptide fibril is shown schematically in Egelman et al. (Structure 2015, 23, 280-289, incorporated herein by reference).

Nanofibers have been observed to be up to several microns long. The nanofiber can have a length of at least, at most, or exactly 100 nm, 200 nm, 300 nm, 400 nm, 500 nm, 600 nm, 700 nm, 800 nm, 900 nm, 1 μm, 1.2 μm, 1.4 μm, 1.6 μm, 1.8 μm, 2 μm, 2.2 μm, 2.4 μm, 2.6 μm, 2.8 μm, 3 μm, 3.2 μm, 3.4 μm, 3.6 μm, 3.8 μm, 4 μm, 4.2 μm, 4.4 μm, 4.6 μm, 4.8 μm, or 5 μm. The nanofiber may be about 100 nm to 1 μm, 100 nm to 2 μm, 100 nm to 3 μm, 100 nm to 4 μm, or 100 nm to 5 μm in length. The nanofiber can have a length of at least, at most, or exactly 0.01, 0.05, 0.1, 0.15, 0.20, 0.25, 0.5, 1, 5, 10, 20, 30, 40, 50, 75, 100, 125, 150, 175, 200, 225, 250, or 300 μm, including all values and ranges there between. In some embodiments, the nanofiber is at least 100, 150, 200, 250, 300, or 350 nanometers in length. In some embodiments, the nanofiber is less than 10, 5, or 2 μm in length. In some embodiments, the nanofiber is 50 nm to 600 nm in length. In certain aspects, the nanofiber has a molecular weight of at least 100, 500, 1,000, 5,000, 10,000, 100,000 Da to 1×10⁶, 1×10⁷, 7×10⁸ Da, including all values and ranges there between. The nanofiber can have a diameter or width of at least, at most, or exactly 5, 10, 15, 20, 25, or 30 nm. In some embodiments, the nanofiber is 5-30 nm in diameter or width.

### 6. Immune Response and Immunoassays

As discussed above, the compositions and methods provided herein include evoking or inducing an immune response in a subject against an antigen. The antigen may comprise an anaphylatoxin epitope. In one embodiment, the immune response can protect against or treat a subject having, suspected of having, or at risk of developing a disease. One use of the immunogenic compositions is to provide effective vaccines and/or active immunotherapies. The compositions detailed herein may induce an immune response. The immune response may be an antigen-specific immune response. In some embodiments, the antigen-specific immune response is temporary or not life-long. In some embodiments, the antigen-specific immune response is long lasting. “Long lasting” may be an antibody response that lasts for at least about 2 weeks, at least about 3 weeks, at least about 4 weeks, at least about 6 weeks, at least about 10 weeks, at least about 12 weeks, at least about 14 weeks, at least about 16 weeks, at least about 18 weeks, at least about 20 weeks, at least about 22 weeks, at least about 24 weeks, at least about 26 weeks, at least about 28 weeks, at least about 30 weeks, at least about 32 weeks, at least about 34 weeks, at least about 36 weeks, at least about 38 weeks, at least about 40 weeks, at least about 42 weeks, at least about 44 weeks, at least about 46 weeks, at least about 48 weeks, at least about 50 weeks, at least about 52 weeks, at least about 1 year, at least about 1.5 years, at least about 2 years, at least about 2.5 years, at least about 3 years, at least about 3.5 years, at least about 4 years, at least about 4.5 years, at least about 5 years, at least about 5.5 years, at least about 6 years, at least about 7 years, at least about 8 years, at least about 9 years, at least about 10 years, at least about 15 years, at least about 20 years, at least about 25 years, at least about 30 years, at least about 35 years, at least about 40 years, at least about 45 years, at least about 50 years, at least about 55 years, at least about 60 years, at least about 65 years, at least about 70 years, at least about 75 years, at least about 80 years, at least about 85 years, at least about 90 years, or at least about 95 years, or a lifetime. “Long lasting” may be an antibody response that lasts for about 3 weeks to a lifetime, about 1 month to a lifetime, about 1 year to a lifetime, about 1 year to about 30 years, or about 5 years to about 20 years. The anti-anaphylatoxin antibodies generated may be IgM, or IgG, or IgA, or a combination thereof. In some embodiments, the antibodies are IgM antibodies. In some embodiments, the antibodies are IgA antibodies. In some embodiments, the antibodies are IgG antibodies. The immune response may include IgG antibody isotypes and subclasses. In some embodiments, the immune response comprises IgG1, IgG2, IgG3, or IgG4 antibody isotypes, or a combination thereof. In some embodiments, the immune response comprises IgM antibody isotypes. In some embodiments, the immune response comprises IgG antibody isotypes. The immunogenic composition may have increased immunogenicity relative to a control. In some embodiments, the control comprises an anaphylatoxin epitope or other antigen without a self-assembling peptide.

The compositions and methods detailed herein may increase the level of anti-anaphylatoxin antibodies relative to a control. The level of anti-anaphylatoxin antibodies may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The level of anti-anaphylatoxin antibodies may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The level of anti-anaphylatoxin antibodies may be increased by about 5-95%, 10-90%, 15-85%, 20-80%, or 1.5-fold to 10-fold, relative to a control.

Further provided herein is the implementation of serological assays to evaluate whether and to what extent an immune response is induced or evoked by compositions. There are many types of immunoassays that can be implemented. Immunoassays include, but are not limited to, those described in U.S. Pat. No. 4,367,110 (double monoclonal antibody sandwich assay) and U.S. Pat. No. 4,452,901 (western blot), which are incorporated herein by reference. Other assays include immunoprecipitation of labeled ligands and immunocytochemistry, both in vitro and in vivo.

Immunoassays generally are binding assays. Certain immunoassays are the various types of enzyme linked immunosorbent assays (ELISAs) and radioimmunoassays (RIA) known in the art. Immunohistochemical detection using tissue sections may also be useful. In one example, antibodies or antigens are immobilized on a selected surface, such as a well in a polystyrene microtiter plate, dipstick, or column support. Then, a test composition suspected of containing the desired antigen or antibody, such as a clinical sample, is added to the wells. After binding and washing to remove non-specifically bound immune complexes, the bound antigen or antibody may be detected. Detection is generally achieved by the addition of another antibody, specific for the desired antigen or antibody, that is linked to a detectable label. This type of ELISA is known as a “sandwich ELISA.” Detection also may be achieved by the addition of a second antibody specific for the desired antigen, followed by the addition of a third antibody that has binding affinity for the second antibody, with the third antibody being linked to a detectable label.

Competition ELISAs are also possible implementations in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal. Irrespective of the format employed, ELISAs have certain features in common, such as coating, incubating or binding, washing to remove non-specifically bound species, and detecting the bound immune complexes.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

a. Protective Immunity

In some embodiments, proteinaceous compositions confer protective immunity to a subject. Protective immunity refers to a body's ability to mount a specific immune response that protects the subject from developing a particular disease or condition that involves the agent against which there is an immune response. An immunogenically effective amount is capable of conferring protective immunity to the subject.

As used herein the phrase “immune response” or its equivalent “immunological response” may refer to the development of a humoral (antibody mediated), cellular (mediated by antigen-specific T cells or their secretion products) or both humoral and cellular response directed against a protein, peptide, carbohydrate, or polypeptide in a recipient patient. Such a response can be an active response induced by administration of immunogen or a passive response induced by administration of antibody, antibody containing material, or primed T-cells. A cellular immune response is elicited by the presentation of polypeptide epitopes in association with Class I or Class II MHC molecules, to activate antigen-specific CD4 (+) T helper cells and/or CD8 (+) cytotoxic T cells. The response may also involve activation of monocytes, macrophages, NK cells, basophils, dendritic cells, astrocytes, microglia cells, eosinophils or other components of innate immunity. As used herein “active immunity” refers to any immunity conferred upon a subject by administration of an antigen.

As used herein “passive immunity” refers to any immunity conferred upon a subject without administration of an antigen to the subject. “Passive immunity” therefore includes, but is not limited to, administration of activated immune effectors including cellular mediators or protein mediators (e.g., monoclonal and/or polyclonal antibodies) of an immune response. A monoclonal or polyclonal antibody composition may be used in passive immunization for the prevention or treatment of infection by organisms that carry the antigen recognized by the antibody. An antibody composition may include antibodies that bind to a variety of antigens that may in turn be associated with various organisms. The antibody component can be a polyclonal antiserum. In certain aspects the antibody or antibodies are affinity purified from an animal or second subject that has been challenged with an antigen(s). Alternatively, an antibody mixture may be used, which is a mixture of monoclonal and/or polyclonal antibodies to antigens for the same, related, or different disease or disorder.

Passive immunity may be imparted to a patient or subject by administering to the patient immunoglobulins (lg) and/or other immune factors obtained from a donor or other non-patient source having a known immunoreactivity. In other aspects, an antigenic composition as detailed herein can be administered to a subject who then acts as a source or donor for globulin, produced in response to challenge with the antigenic composition (“hyperimmune globulin”), that contains antibodies directed against an anaphylatoxin epitope, for example. A subject thus treated would donate plasma from which hyperimmune globulin would then be obtained, via conventional plasma-fractionation methodology, and administered to another subject in order to impart resistance against or to treat inflammation, for example. Hyperimmune globulins are particularly useful for immune-compromised individuals, for individuals undergoing invasive procedures or where time does not permit the individual to produce their own antibodies in response to vaccination. See U.S. Pat. Nos. 6,936,258, 6,770,278, 6,756,361, 5,548,066, 5,512,282, 4,338,298, and 4,748,018, each of which is incorporated herein by reference in its entirety, for exemplary methods and compositions related to passive immunity.

For purposes of this specification and the accompanying claims the terms “epitope” and “antigenic determinant” are used interchangeably to refer to a site on an antigen to which B and/or T cells respond or recognize. B-cell epitopes can be formed from small molecules. For example, B-cell epitopes may be formed from an anaphylatoxin epitope. B-cell epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols (1996), incorporated herein by reference. Antibodies that recognize the same epitope can be identified in a simple immunoassay showing the ability of one antibody to block the binding of another antibody to a target antigen. T-cells recognize continuous epitopes of about nine amino acids for CD8 cells or about 13-15 amino acids for CD4 cells. T cells that recognize the epitope can be identified by in vitro assays that measure antigen-dependent proliferation, as determined by ³H-thymidine incorporation by primed T cells in response to an epitope (Burke et al., 1994, incorporated herein by reference), by antigen-dependent killing (cytotoxic T lymphocyte assay, Tigges et al., 1996, incorporated herein by reference) or by cytokine secretion.

The presence of a cell-mediated immunological response can be determined by proliferation assays (CD4 (+) T cells) or CTL (cytotoxic T lymphocyte) assays. The relative contributions of humoral and cellular responses to the protective or therapeutic effect of an immunogen can be distinguished by separately isolating IgG and T-cells from an immunized syngeneic animal and measuring protective or therapeutic effect in a second subject.

As used herein and in the claims, the terms “antibody” or “immunoglobulin” are used interchangeably and refer to any of several classes of structurally related proteins that function as part of the immune response of an animal or recipient, which proteins include IgG, IgD, IgE, IgA, IgM, and related proteins.

Under normal physiological conditions antibodies are found in plasma and other body fluids and in the membrane of certain cells and are produced by lymphocytes of the type denoted B cells or their functional equivalent. Antibodies of the IgG class are made up of four polypeptide chains linked together by disulfide bonds. The four chains of intact IgG molecules are two identical heavy chains referred to as H-chains and two identical light chains referred to as L-chains.

In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific. Monoclonal antibodies can be produced by hyperimmunization of an appropriate donor with the antigen or ex-vivo by use of primary cultures of splenic cells or cell lines derived from spleen (Anavi, 1998; Huston et al., 1991; Johnson et al., 1991; Mernaugh et al., 1995, each incorporated herein by reference).

As used herein, the phrase “an immunological portion of an antibody” includes a Fab fragment of an antibody, a Fv fragment of an antibody, a heavy chain of an antibody, a light chain of an antibody, a heterodimer consisting of a heavy chain and a light chain of an antibody, a variable fragment of a light chain of an antibody, a variable fragment of a heavy chain of an antibody, and a single chain variant of an antibody, which is also known as scFv. In addition, the term includes chimeric immunoglobulins which are the expression products of fused genes derived from different species, one of the species can be a human, in which case a chimeric immunoglobulin is said to be humanized. Typically, an immunological portion of an antibody competes with the intact antibody from which it was derived for specific binding to an antigen.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

### 7. Pharmaceutical Compositions

Further provided herein are pharmaceutical compositions comprising the anaphylatoxin peptide conjugate or nanofibers detailed herein. Compositions can include a peptide fibril coupled to a plurality of antigens such as an anaphylatoxin epitope, and may be referred to as a “fibril complex.” In some embodiments, the composition does not further comprise an adjuvant. In some embodiments, the composition further comprises an adjuvant. In some embodiments, the peptide fibril is an adjuvant.

The preparation of pharmaceutical compositions such as vaccines that contain polypeptide or peptide sequence(s) as active ingredients is generally well understood in the art, as exemplified by U.S. Pat. Nos. 4,608,251; 4,601,903; 4,599,231; 4,599,230; 4,596,792; and 4,578,770, all of which are incorporated herein by reference. Typically, such pharmaceutical compositions are prepared as injectables either as liquid solutions or suspensions: solid forms suitable for solution in or suspension in liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, or the like and combinations thereof. In addition, if desired, the vaccine may contain amounts of auxiliary substances such as wetting or emulsifying agents, pH buffering agents, or adjuvants that enhance the effectiveness of the vaccines. In specific embodiments, pharmaceutical compositions are formulated with a combination of substances, as described in U.S. Pat. Nos. 6,793,923 and 6,733,754, which are incorporated herein by reference.

Pharmaceutical compositions may be conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides: such suppositories may be formed from mixtures containing the active ingredient in the range of about 0.5% to about 10%, preferably about 1% to about 2%. Oral formulations include such normally employed excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders and contain about 10% to about 95% of active ingredient, preferably about 25% to about 70%.

In some embodiments, the compositions described herein are formulated for sublingual administration. The compositions may be formulated into a tablet. For example, the compositions may be formulation into a dissolving tablet. The dissolving tablet may be placed under the tongue. The compositions may be formulated into a table by a variety of suitable means, for example, the compositions may be freeze dried and pressed into a tablet form.

The compositions described herein may be formulated into a pharmaceutical composition as neutral or salt forms. Pharmaceutically-acceptable salts include the acid addition salts (formed with the free amino groups of the peptide) and those that are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Typically, compositions are administered in a manner compatible with the dosage formulation, and in such amount as will be therapeutically effective and immunogenic. The quantity to be administered depends on the subject to be treated, including the capacity of the individual's immune system to synthesize antibodies and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable, but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection and the like. The dosage of the vaccine will depend on the route of administration and will vary according to the size and health of the subject.

The compositions and related methods, particularly administration of a peptide conjugate or nanofiber, may also be used in combination with the administration of traditional therapies. These include, but are not limited to, the administration of antibiotics such as streptomycin, ciprofloxacin, doxycycline, gentamycin, chloramphenicol, trimethoprim, sulfamethoxazole, ampicillin, tetracycline or various combinations of antibiotics.

In one aspect, it is contemplated that a peptide conjugate or nanofiber or pharmaceutical composition is used in conjunction with an additional treatment described herein. Alternatively, the therapy may precede or follow the other agent treatment by intervals ranging from minutes to weeks. In embodiments where the other agents and/or a proteins is administered separately, one may generally ensure that a significant period of time did not expire between the time of each delivery, such that the agent and antigenic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly, however, where several days (2, 3, 4, 5, 6, or 7) to several weeks (1, 2, 3, 4, 5, 6, 7, or 8) lapse between the respective administrations.

Various combinations may be employed, for example antibiotic therapy is “A” and the immunogenic composition is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the pharmaceutical compositions to a patient/subject will follow general protocols for the administration of such compounds, taking into account the toxicity, if any. It is expected that the treatment cycles would be repeated as necessary. It also is contemplated that various standard therapies, such as hydration, may be applied in combination with the described therapy.

In some embodiments, pharmaceutical compositions are administered to a subject. Different aspects involve administering an effective amount of a composition to a subject. In some embodiments, immunogenic compositions may be administered to the patient to protect against disease and/or inflammation. Additionally, such compounds can be administered in combination with an anti-inflammatory, a pain reliever, or other therapy.

Such compositions will generally be dissolved or dispersed in a pharmaceutically acceptable carrier or aqueous medium. The use of such media and agents for pharmaceutical active substances is well known in the art. Except insofar as any conventional media or agent is incompatible with the active ingredients, its use in immunogenic and therapeutic compositions is contemplated. Supplementary active ingredients can also be incorporated into the compositions.

In addition to the compounds formulated for parenteral administration, such as those for intravenous or intramuscular injection, other pharmaceutically acceptable forms include, e.g., tablets or other solids for oral administration; time release capsules; and any other form currently used, including creams, lotions, mouthwashes, inhalants and the like.

The active compounds can be formulated for parenteral administration, e.g., formulated for injection via the intravenous, intramuscular, sub-cutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Administration of the compositions will typically be via any common route. This includes, but is not limited to oral, nasal, or buccal administration. Alternatively, administration may be by orthotopic, intradermal, subcutaneous, intramuscular, intraperitoneal, intranasal, or intravenous injection. In certain embodiments, a vaccine composition may be inhaled (e.g., U.S. Pat. No. 6,651,655, which is specifically incorporated by reference). Such compositions would normally be administered as pharmaceutically acceptable compositions that include physiologically acceptable carriers, buffers or other excipients. In some embodiments, the composition is administered to the subject intravenously, intraarterially, intraperitoneally, subcutaneously, intranasally, intramuscularly, or intratumorally. In some embodiments, the immunogenic composition is administered orally. In some embodiments, the immunogenic composition is administered sublingually.

For parenteral administration in an aqueous solution, for example, the solution may be suitably buffered, if necessary, and the liquid diluent first rendered isotonic with sufficient saline or glucose. These particular aqueous solutions are especially suitable for intravenous, intramuscular, subcutaneous, and intraperitoneal administration. In this connection, sterile aqueous media which can be employed will be known to those of skill in the art in light of the present disclosure. For example, one dosage could be dissolved in isotonic NaCl solution and either added to hypodermoclysis fluid or injected at the proposed site of infusion, (see for example, Remington's Pharmaceutical Sciences, 1990). Some variation in dosage will necessarily occur depending on the condition of the subject. The person responsible for administration will, in any event, determine the appropriate dose for the individual subject.

An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired.

Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions may be administered in a manner compatible with the dosage formulation and in such amount as is therapeutically or prophylactically effective. The formulations may be easily administered in a variety of dosage forms, such as the type of injectable solutions described above.

In some embodiments, the pharmaceutical compositions comprising the anaphylatoxin peptide conjugate or nanofibers detailed herein further include at least one additional therapeutic agent. The at least one additional therapeutic agent may comprise antibiotics, NSAIDS, anti-inflammatory compounds, hemoperfusion devices, quorum sensing inhibitors, lytic bacteriophage, polyclonal or monoclonal antibodies, non-immune tolerizing approaches, liposome-based cytotoxin inhibitors, or combinations thereof. The at least one additional therapeutic agent may comprise an adjuvant. Suitable adjuvants may be obtained commercially. In some embodiments, the adjuvant comprises CpG, cholera toxin B subunit (CTB), STING agonists like cyclic dinucleotides such as cyclic-di-AMP and/or cyclic GMP-AMP (cGAMP), retinoic acid, heat labile toxin B subunit, alum, MF59, 3M-052, iscomatrix, squalene-based adjuvants, AS01, AS03, or AS04, or a combination thereof.

### 8. Methods

a. Methods of Treating an Inflammatory Condition

Further provided herein are methods of treating an inflammatory condition. Further provided herein are methods of treating or reducing inflammation. The methods may include administering to a subject an anaphylatoxin conjugate peptide, or nanofiber, or pharmaceutical composition as detailed herein. The anaphylatoxin conjugate peptide, or nanofiber, or pharmaceutical composition may be administered intraperitoneally, orally, sublingually, intravenously, nasally, buccally, transdermally, intranasally, or intramuscularly. In some embodiments, the anaphylatoxin conjugate peptide, or nanofiber, or pharmaceutical composition is administered sublingually.

In some embodiments, the inflammatory condition comprises inflammatory bowel disease (IBD), sepsis, ulcerative colitis, Crohn's disease, rheumatoid arthritis, psoriasis, cirrhosis, atherosclerosis, cardiovascular inflammation, lupus, multiple sclerosis, Alzheimer's disease, pulmonary fibrosis, immunosenescence, or a combination thereof. The inflammation may be chronic or acute.

In some embodiments, method further comprises administering at least one additional therapeutic agent. The at least one additional therapeutic agent may be administered prior to the anaphylatoxin conjugate peptide or the nanofiber or the pharmaceutical composition, or concurrently with the anaphylatoxin conjugate peptide or the nanofiber or the pharmaceutical composition, or after the anaphylatoxin conjugate peptide or the nanofiber or the pharmaceutical composition. The at least one additional therapeutic agent may comprise antibiotics, NSAIDS, anti-inflammatory compounds, hemoperfusion devices, quorum sensing inhibitors, lytic bacteriophage, polyclonal or monoclonal antibodies, non-immune tolerizing approaches, liposome-based cytotoxin inhibitors, or combinations thereof. The at least one additional therapeutic agent may comprise an adjuvant. In some embodiments, the adjuvant comprises CpG, cholera toxin B subunit (CTB), STING agonists like cyclic dinucleotides such as cyclic-di-AMP and/or cyclic GMP-AMP (cGAMP), retinoic acid, or heat labile toxin B subunit, or a combination thereof.

Upon or after administration, inflammation may be treated or reduced. The inflammation may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The inflammation may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The inflammation be reduced by about 5-95%, 10-90%, 15-85%, 20-80%, or 1.5-fold to 10-fold, relative to a control.

In some embodiments, the subject forms an antibody to the anaphylatoxin epitope. Further provided herein is an antibody produced in the immune response.

b. Methods of Treating Sepsis

Further provided herein are methods of treating sepsis. The methods may include administering to the subject an anaphylatoxin conjugate peptide, or nanofiber, or pharmaceutical composition as detailed herein. In some embodiments, the anaphylatoxin peptide conjugate or the nanofiber is administered to the subject intraperitoneally, orally, sublingually, intravenously, nasally, buccally, transdermally, intranasally, or intramuscularly. In some embodiments, the anaphylatoxin conjugate peptide, or nanofiber, or pharmaceutical composition is administered sublingually.

In some embodiments, method further comprises administering at least one additional therapeutic agent. The at least one additional therapeutic agent may be administered prior to the anaphylatoxin conjugate peptide or the nanofiber or the pharmaceutical composition, or concurrently with the anaphylatoxin conjugate peptide or the nanofiber or the pharmaceutical composition, or after the anaphylatoxin conjugate peptide or the nanofiber or the pharmaceutical composition. The at least one additional therapeutic agent may comprise an adjuvant. In some embodiments, the adjuvant comprises CpG, cholera toxin B subunit (CTB), STING agonists like cyclic dinucleotides such as cyclic-di-AMP and/or cyclic GMP-AMP (cGAMP), retinoic acid, or heat labile toxin B subunit, or a combination thereof.

Upon or after administration, sepsis may be treated or reduced. The sepsis may be reduced by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The sepsis may be reduced by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The sepsis be reduced by about 5-95%, 10-90%, 15-85%, 20-80%, or 1.5-fold to 10-fold, relative to a control.

In some embodiments, the subject forms an antibody to the anaphylatoxin epitope. Further provided herein is an antibody produced in the immune response.

### 9. Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Materials and Methods

Epitope selection. Previously, three C5a epitopes were identified using the Kolaskar Tongaonkar Antigenicity Test (KTAT), which uses a semi-empirical method based on physicochemical characteristics of amino acids and their frequency in antigenic epitopes to determine immunogenic peptides within a protein sequence with 75% accuracy (Kolaskar and Tongaonkar, FEBS Letters, 1990; 276:172-174). Two out of the three predicted sequences, named pC5a1 and pC5a3 arbitrarily, were immunogenic in vivo and selected for this study (Hainline et al., Acta Biomaterialia, 2024, S1742-7061 (24) 00112-0). Three additional C3a epitopes were predicted with the KTAT and named pC3a1, pC3a2, and pC3a3.

Peptide synthesis and purification. Synthetic peptides (TABLE 1) were synthesized using microwave-assisted solid-phase peptide synthesis on Rink amide resin and cleaved using a 95% TFA 2.5% triisopropylsilane and 2.5% water cocktail for two hours at room temperature (i.e., 20-22° C.). Peptides were precipitated and washed in diethyl ether, purified using a C12 Waters XBridge column by reverse-phase HPLC, and fractions were frozen and lyophilized. MALDI-TOF mass spectrometry was used to confirm peptide mass and identity. Peptides were stored as dry powders at about-20° C.

Nanofiber preparation. All immunizations contained a total peptide concentration of 2 mM. PADRE-containing formulations were composed of 1 mM C5a-Coil29, 0.05 mM PADRE-Coil29, and 0.95 mM Coil29. For single-epitope formulations, nanofibers were made by coassembly of 1 mM C3aX or C5aX-Coil29 with 1 mM Coil29. DoE and dual-epitope formulations are described below, but briefly they contained a range of 0 mM to 1.19 mM of each epitope, with Coil29 backbone supplementing a final concentration of 2 mM. Peptides were dissolved at a total concentration of 2.22 mM in 10 mM acetate buffer pH 4. The solution was heated to 95° C. for 10 minutes and then annealed to room temperature. After cooling, the concentration was brought down to 2 mM with 10×PBS and incubated at room temperature overnight.

Atomic Force Microscopy (AFM). Nanofibers were prepared for imaging as previously described (Fries et al., Advanced Materials, 2020; 32:2003310). Briefly, nanofibers were diluted in water to a 0.2 mM concentration and mica substrates (Ted Pella, Inc.) were cleaved immediately before use. 20 μL of the nanofiber solution was dropped onto the mica surface and incubated for 30 seconds. Substrates were rinsed with ultrapure water and dried under a stream of nitrogen. Imaging was conducted using a Bruker AFM on tapping mode with RTESPA-300 silicon tips.

Nanofiber trafficking. Mice were given 40 μL of PBS, nanofibers containing 10% TAMRA-Coil29 with 10 μg/dose CAMP, or unlabeled Coil29 nanofibers with 10 μg/dose 2′-Fluo-AHC-c-diAMP (FITC-CAMP, BioLog). At 0, 1, 3, 6, 12, and 24 hours, NALT, lungs, cervical lymph nodes, brain, and vital organs were harvested and imaged using an IVIS Lumina XR with 500-550 nm excitation passband and a DsRed filter for TAMRA and with 445-490 nm excitation passband and GFP filter for FITC. The radiant efficiency in each organ was quantified using Perkin Elmer Living Imaging software, and the same scale was set based on control mice for all timepoints.

Mice and immunizations. Female C57BL/6 mice (Envigo) aged 8 weeks were used to start experiments. Nanofibers were prepared as described above and 10 μg/dose of CAMP was added to each formulation. For intranasal immunization, mice were anesthetized and given 40 μL of peptide nanofiber solution via nostrils. Immunizations were administered at weeks 0, 2, and 4, as well as one week before the endpoint in immunogenicity studies. All animal experiments were conducted at Duke University under a protocol approved by the Duke Institutional Animal Care and Use Committee (IAUCUC).

Peptide and Proteins Enzyme-Linked Immunosorbent Assay (ELISA). Peptide and protein ELISAs were performed as previously described (Hainline et al., Acta Biomaterialia, 2024, S1742-7061 (24) 00112-0). Briefly, half-area high binding plates were coated with 20 μg/mL peptide epitopes for peptide ELISAs or 2 μg/mL anti-His tag antibody (Jackson Immunoresearch) for protein ELISAs in PBS overnight at 4° C. For protein ELISAs, anti-His tag antibody coated plates were coated the following day with C3a-His or C5a-His (Antibodies Online and Acro Biosystems) in PBS for 1 hour. Background wells were coated with PBS. Plates were washed with 0.05% Tween 20 in PBS (PBST) and then blocked with SuperBlock (Thermofisher). Serum was serially diluted in BSA-PBST from 1:102 to 1:106 for IgG ELISAs and from 1:52 to 1:56 for IgA ELISAs and applied to coated wells for 2 hours at room temperature. Detection antibodies were applied to wells for 45 minutes at room temperature. HRP conjugated Fcγ fragment specific goat anti-mouse IgG (Jackson Immunoresearch) at a 1:5000 dilution was used to detect total IgG, and SBA Clonotyping System-C57BL/6-HRP (Southern Biotech) for detection of total IgA (1:1000 dilution) and IgG subclasses (1:5000 dilution). Plates were developed with TMB substrate and absorbance was measured at 450 nm with a plate reader with SoftMax Pro 6.5.1 software. Antibody titer was defined as the highest dilution that produced a signal higher than 0.2 after background subtraction. Titer 1 indicates no measurable antibody titer. For IgA ELISAs, absorbance at 450 nm for the lowest dilution was plotted.

Fecal sample processing. Fecal samples were processed for IgG and IgA detection. 10 μL per 1 mg of fecal mass of a solution containing 10% goat serum and 0.01% sodium azide was added to samples, which were incubated on a shaker for 30 minutes at room temperature. Samples were centrifuged at 17,000 g for 15 minutes and the supernatant was collected. IgG and IgA were detected via ELISA as described above. No dilution was performed, and absorbance values at 450 nm after background subtraction are reported.

Nasal-Associated Lymphoid Tissue (NALT) and Peyer's patches culture. NALT and Peyer's patches were harvested at experimental endpoints and cultured for 48 hours at 37° C. Media was collected and IgG and IgA were quantified via ELISA as described above. No dilution was performed, and absorbance values at 450 nm after background subtraction are reported.

Enzyme-Linked Immunospot (ELISpot). T-cell responses were measured using ELISpot. Mice were boosted 6 days prior to euthanasia, spleens were harvested, and splenocytes were isolated using Lympholyte-M (Cedarlane). Cells were stimulated with media as a negative control, Concanavalin A as a positive control (Sigma), C3a peptide epitopes, or Coil29 at 1 μM, and plated at 250,000 cells per well on 96-well ELISpot plates (Millipore) coated with IL-4 or IFNγ capture antibodies at a 1:100 dilution. IL-4 (BD Biosciences) and IFNγ (BD Biosciences) detection antibodies at a 1:250 dilution were used to develop cell spots, followed by addition streptavidin-alkaline phosphatase (Mabtech) and Sigmafast BCIP/NBT (Sigma) substrate. Cell spot quantification was performed by Zellnet Consulting Services. Counts shown represent the number of spots in stimulating conditions after subtraction of spots in negative control wells.

Lipopolysaccharide (LPS) challenge. LPS challenge was performed as described previously (Mora-Solano et al., Biomaterials, 2017; 149:1-11; Hainline et al., Acta Biomaterialia, 2024, S1742-7061 (24) 00112-0). Briefly, mice were challenged with a 10 mg/kg dose of LPS from Escherichia coli 055: B5 (Sigma) in sterile PBS at a concentration of 2 mg/mL. Rectal temperature and body weight were measured 0, 3, 6, 12, 24, 48, and 72 hours after administration. Animals were sacrificed at the end of the experiment or if they reached humane endpoints of >20% weight loss, significant hypothermia indicated by rectal temperatures below 32° C., or inability to eat, drink or ambulate. Survival and maximum temperature changes were reported.

Statistical analysis. Data analysis was conducted using GraphPad Prism software. Figures display mean±standard error of mean (SEM) as error bars. Sample sizes as well as P values and their significance are indicated in the Brief Description of the Drawings for each figure. One-way analysis of variance (ANOVA) was performed to compare one factor in two or more groups. Two-way ANOVA was performed to compare two factors in two or more group, such as antibody titer over time. Tukey's post-hoc multiple comparison test was used for significant main effect. For design and statistical analysis of multifactorial experiments, JMP14 Pro software was used to determine power calculations, surface plots and predictions of antibody titers based on experimental data.

### Example 2

### Nanofiber and Adjuvant Trafficking Following Intranasal Administration

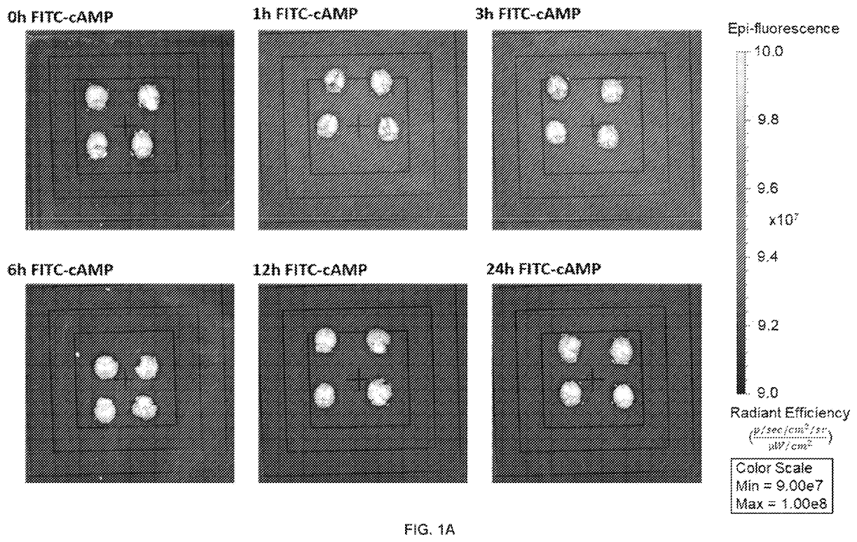

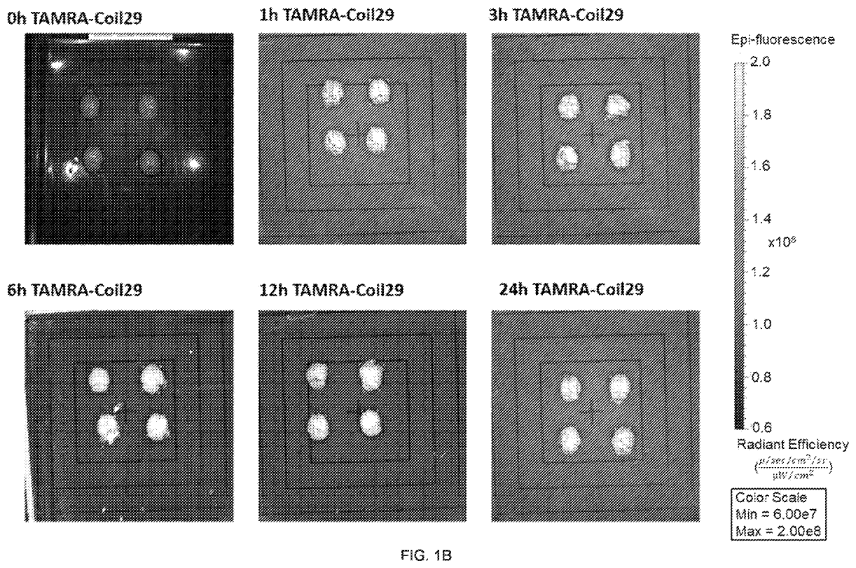

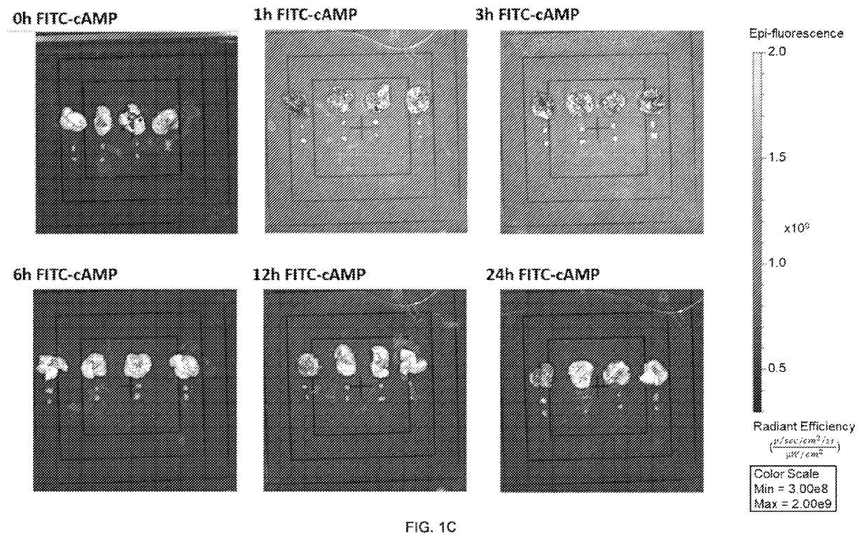

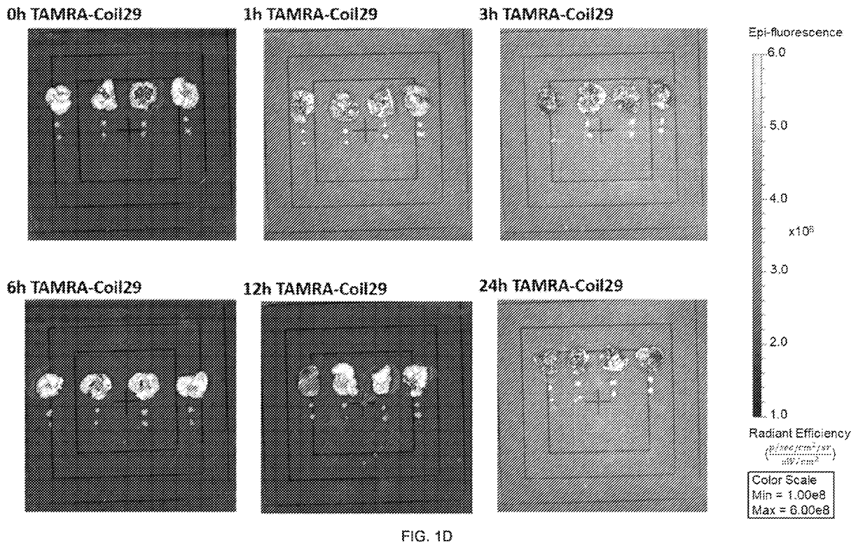

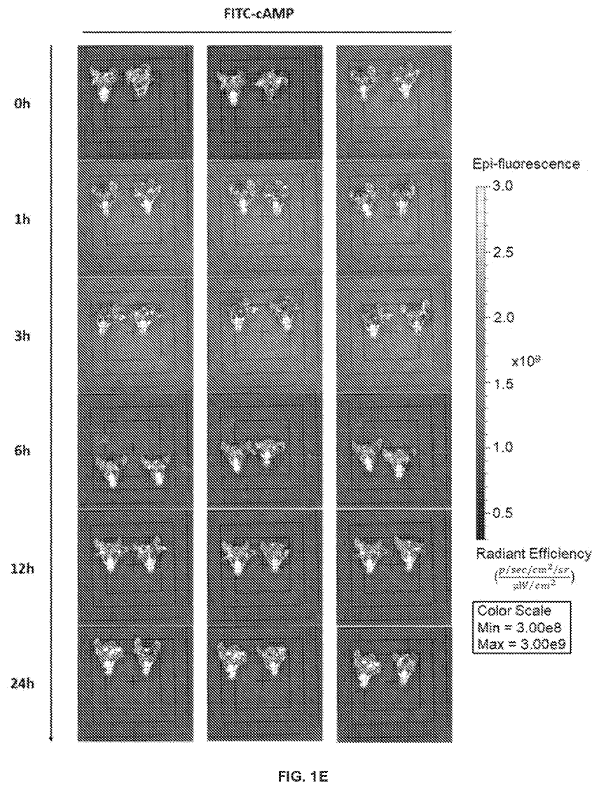

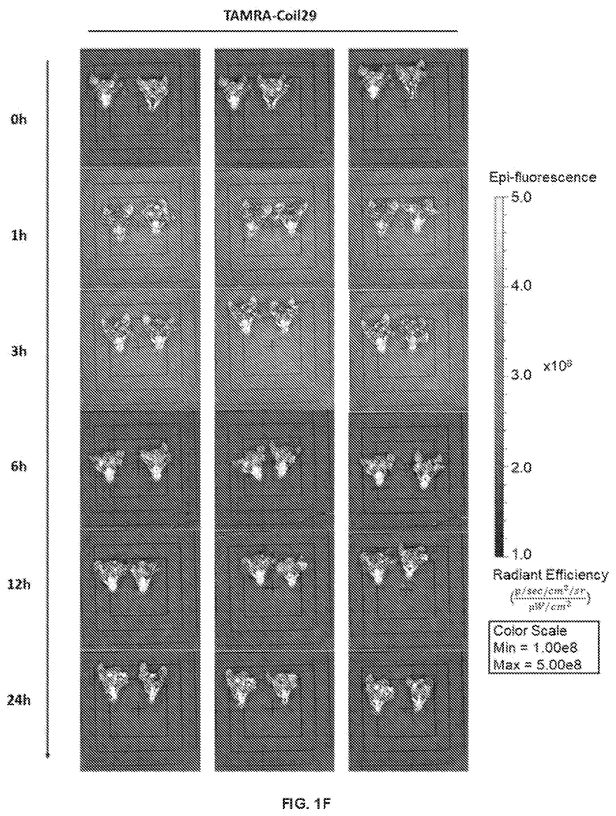

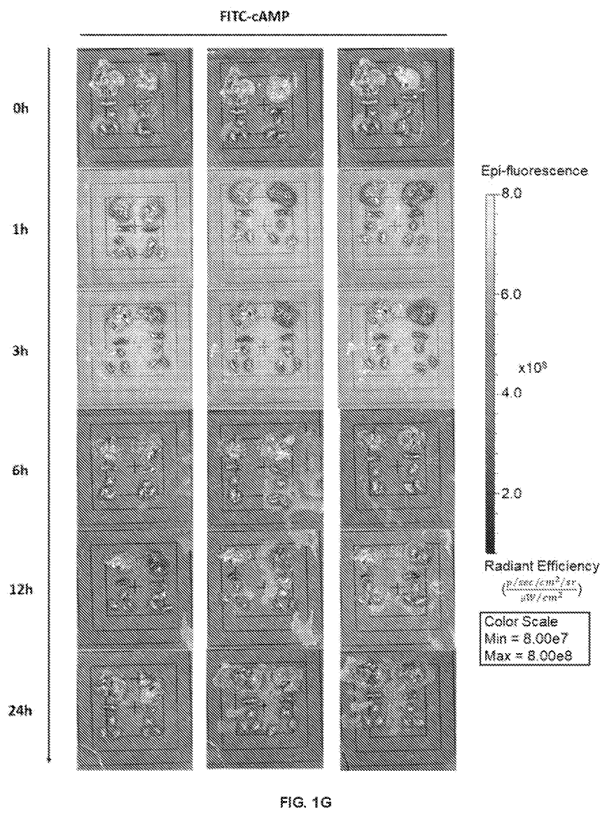

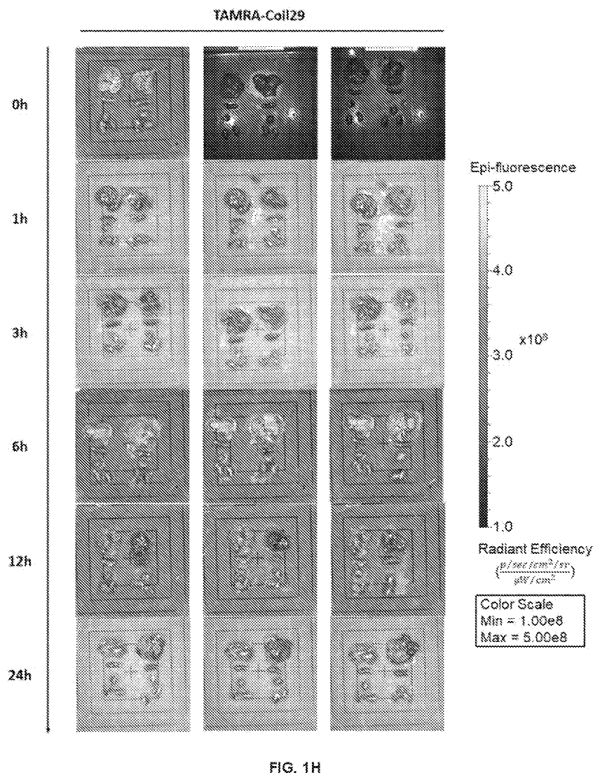

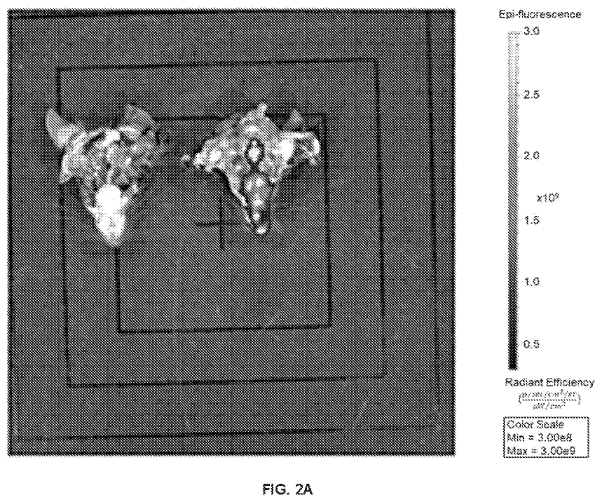

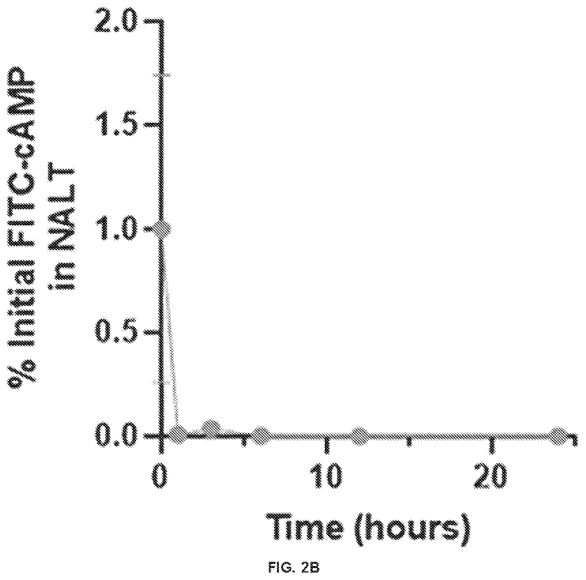

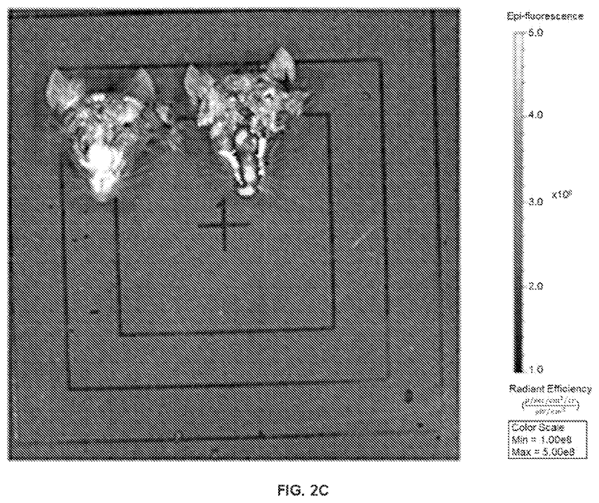

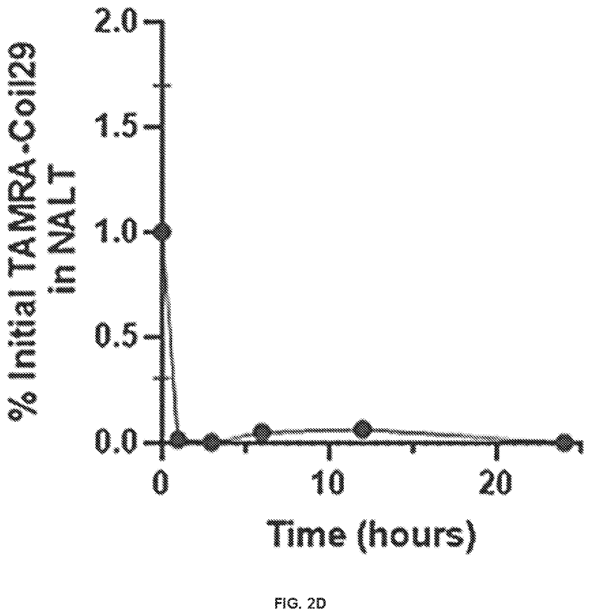

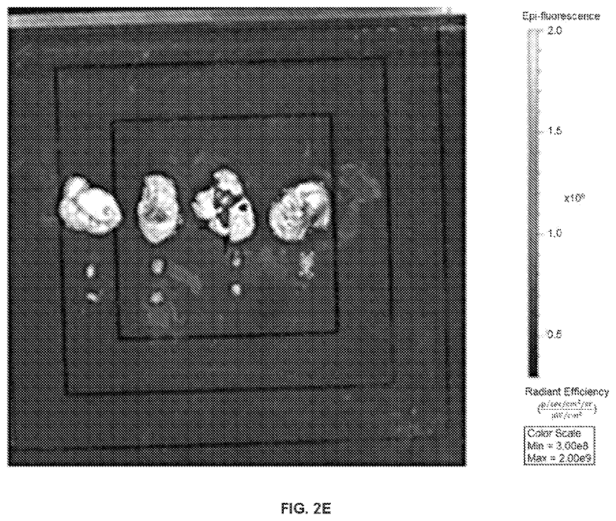

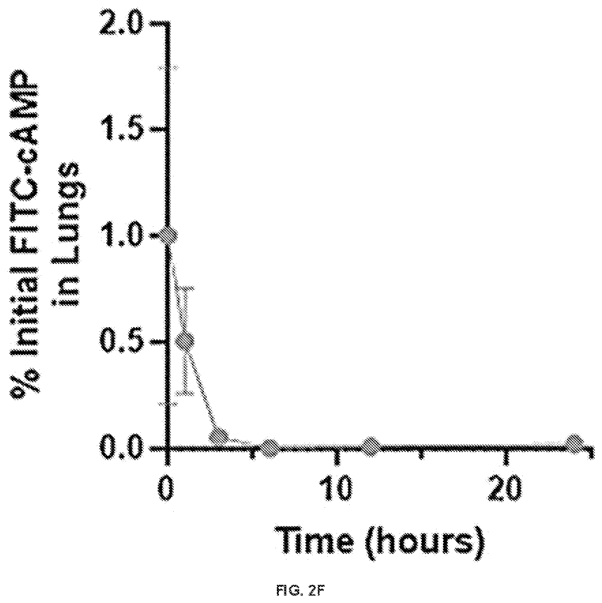

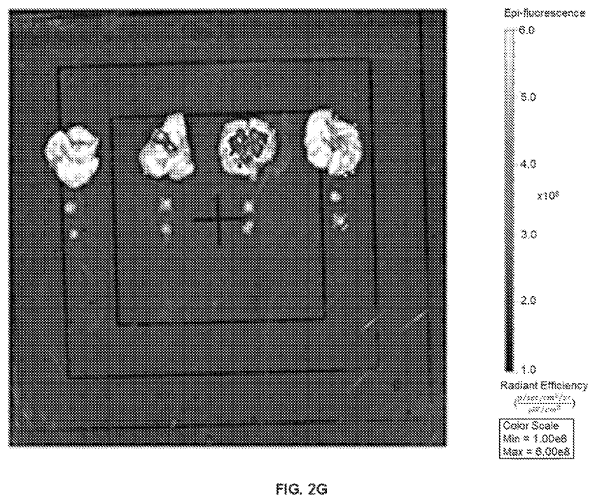

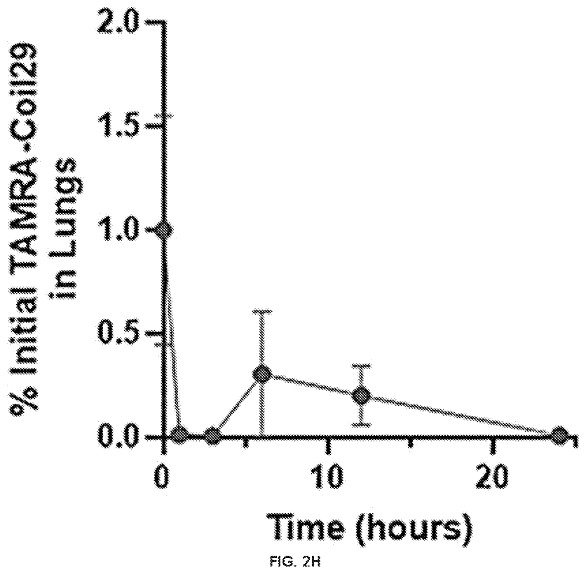

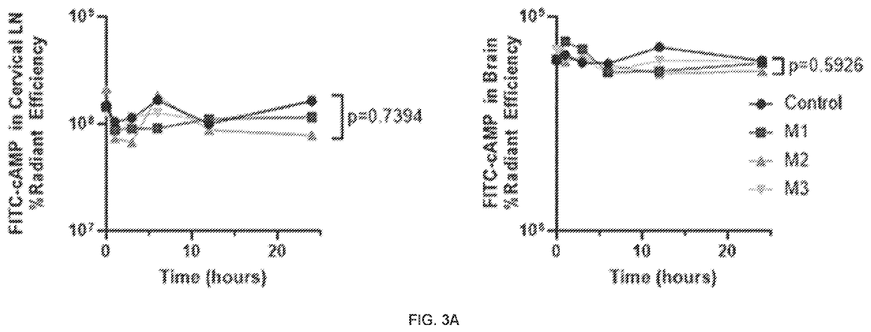

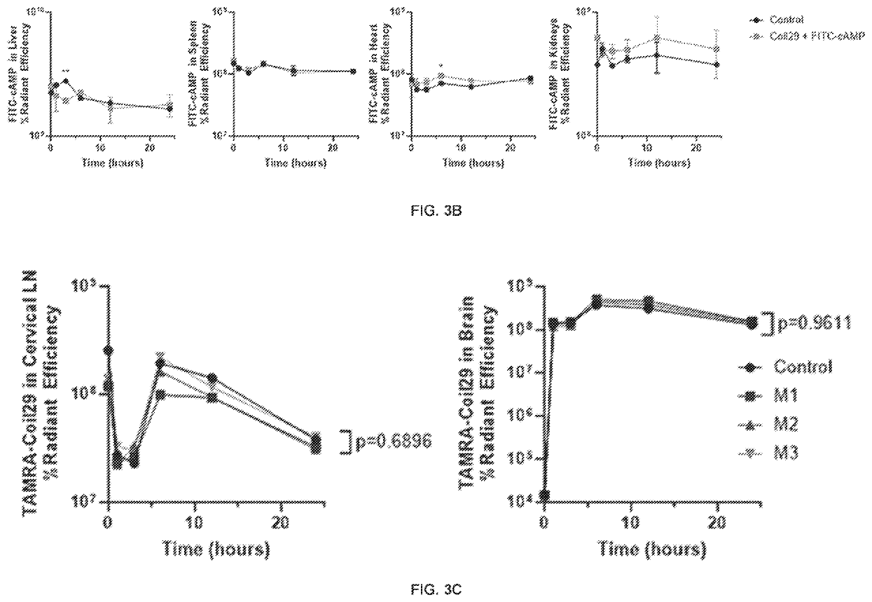

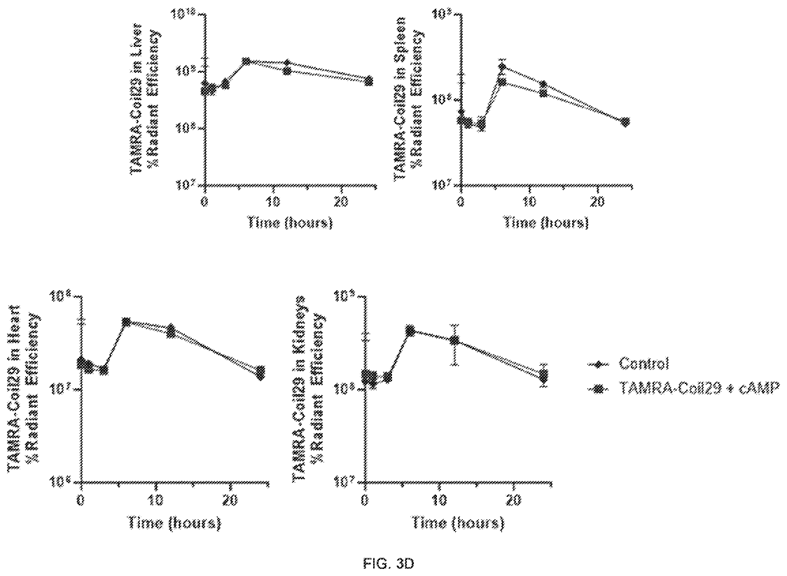

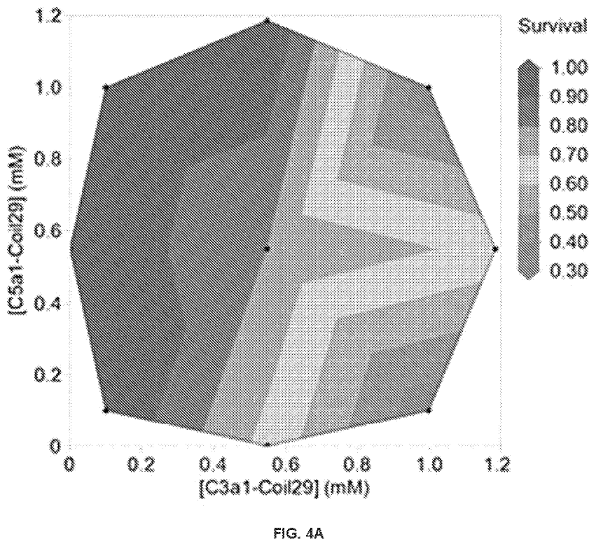

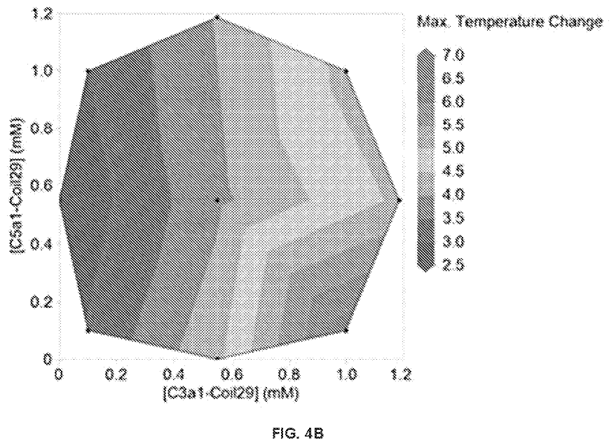

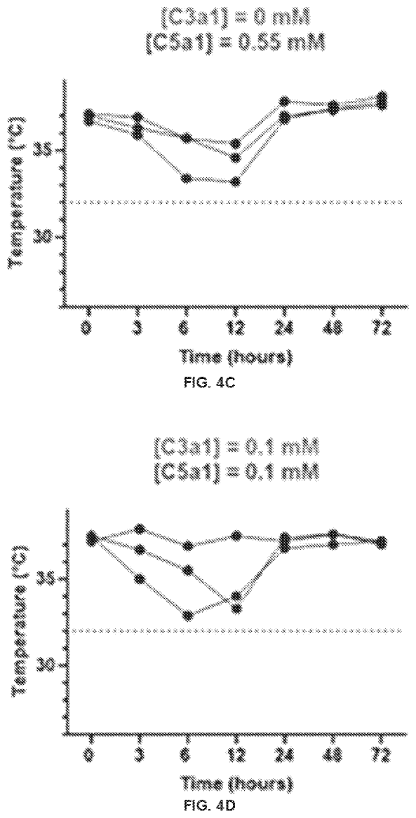

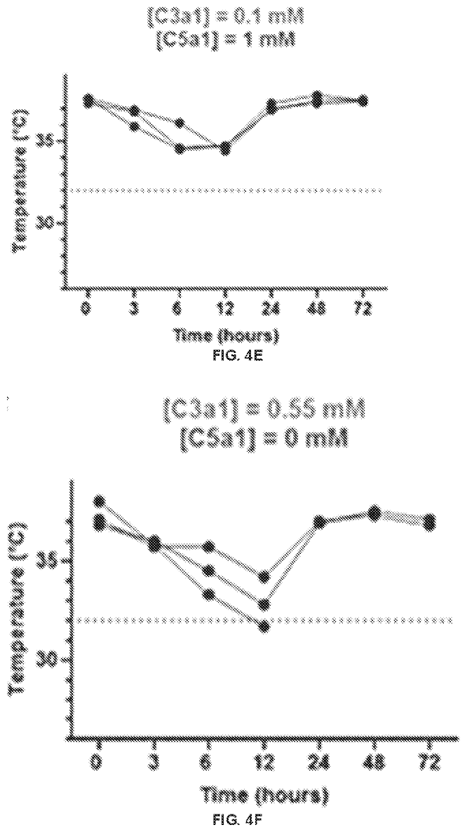

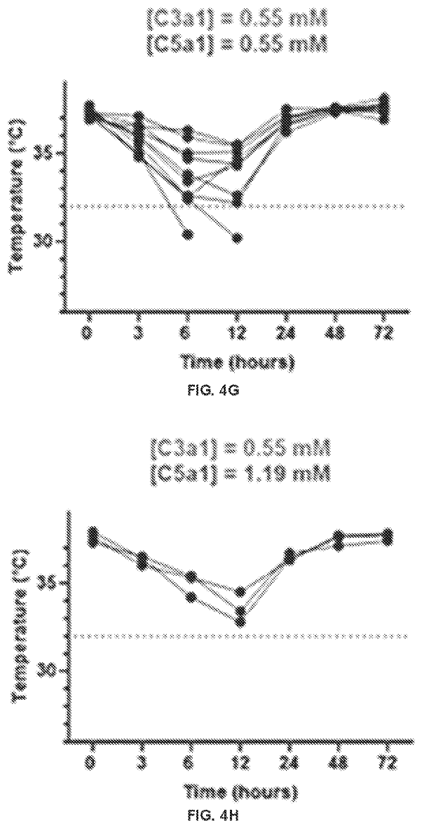

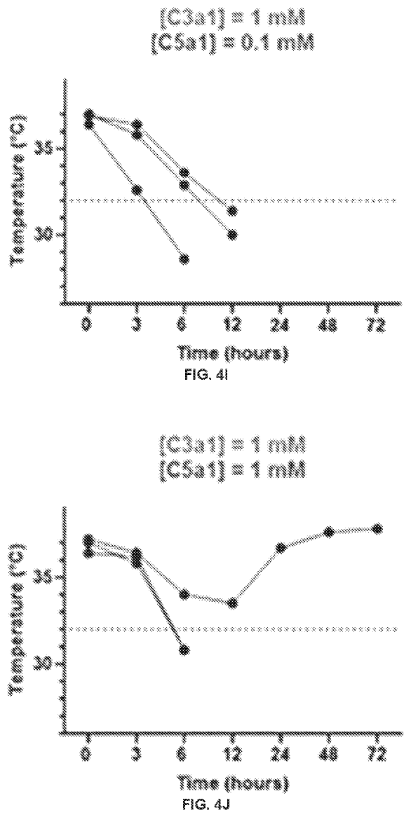

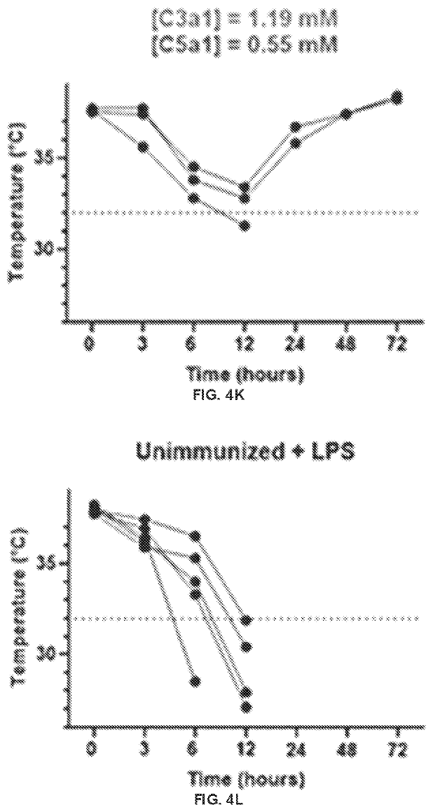

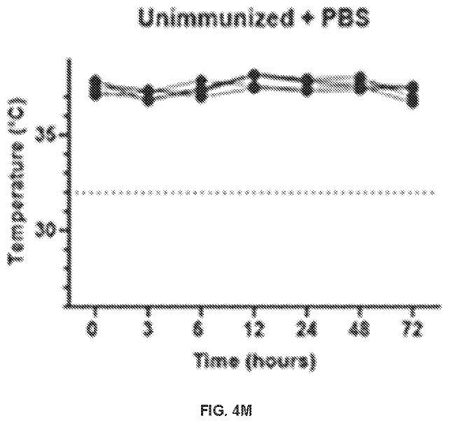

The intranasal route has been chosen for nanofiber administration. Intranasal immunization has been extensively studied in the context of infectious diseases. Due to its ability to raise antibody and cellular immune responses at mucosal tissues, this route of administration has shown promise in vaccines against influenza, SARS-COV-2, and even gastrointestinal pathogens. Immunity after intranasal immunization has been observed not only in the respiratory, but also the gastrointestinal tract via the gut-lung axis. To better understand the trafficking of both adjuvant and nanofibers after intranasal administration, a biodistribution study was performed with IVIS imaging. Animals were immunized with either FITC-labeled cAMP with unlabeled Coil29 nanofibers or TAMRA-labeled Coil29 with unlabeled cAMP and fluorescence was monitored in the NALT, lungs, cervical lymph nodes, brain, and vital organs for 24 hours (FIGS. 1A-1H and FIGS. 3A-3D). Upon administration, FITC-CAMP accumulated in the NALT (FIG. 2A), but the adjuvant was cleared only one hour later (FIG. 2B). The same was observed for TAMRA-Coil29 in the NALT (FIGS. 2C-D). A slightly longer residence time was observed in the lungs. FITC-CAMP was detected in the lungs promptly after administration (FIG. 2E) and had a detectable signal until three hours later (FIG. 2F). For TAMRA-Coil29, fibers were immediately delivered to the lungs (FIG. 2G) and were cleared by the 24-hour mark (FIG. 2H). No significant trafficking was observed to the cervical lymph nodes, brain, or vital organs (FIGS. 4A-M). These results indicated co-delivery of nanofibers and adjuvant to the NALT and lungs and addressed safety concerns by showing no significant trafficking to the brain or other vital organs.

### Example 3

Intranasal Immunization with C5a Epitopes on Coil29 Nanofibers

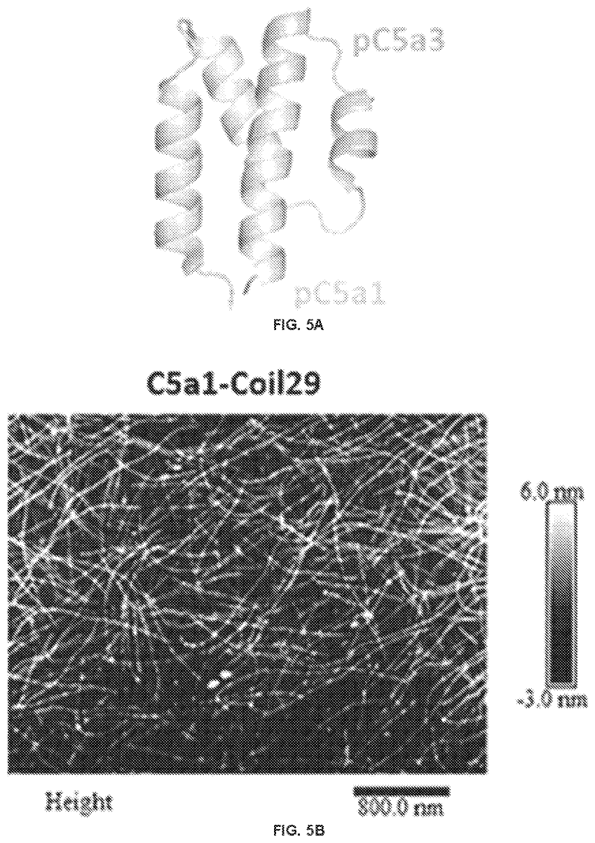

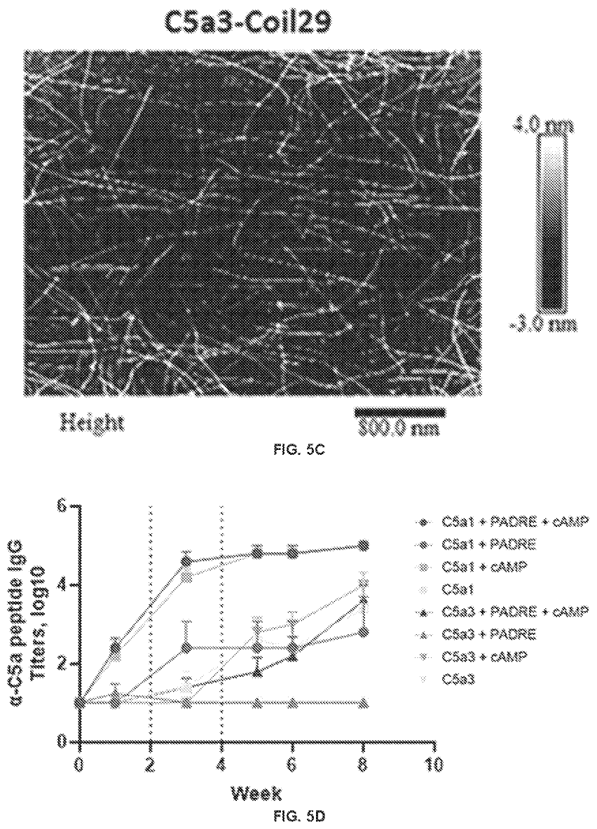

Leveraging the ability to coassemble different B-cell epitopes onto Coil29 nanofibers, this platform was used to raise immune responses against complement anaphylatoxins. Previously, two immunogenic C5a B-cell epitopes were identified subcutaneously on the Q11 platform, pC5a1 and pC5a3 (FIG. 5A). The epitopes were synthesized on the N-terminus of Coil29, and peptides were allowed to assemble to form C5a1-Coil29 and C5a3-Coil29 nanofibers. Nanofiber formation and morphology was confirmed via AFM (FIGS. 5B-5C). Mice were immunized with formulations containing either epitope with or without coassembly with the exogenous T-cell epitope PADRE and with and without the addition of the mucosal STING agonist cyclic-di-AMP (CAMP). IgG titers were monitored for eight weeks (FIG. 5D). While both C5a1-Coil29 and C5a3-Coil29 raised IgG against the adjuvanted peptide, only C5a1-Coil29 raised antibody responses against the entire C5a protein (FIG. 5E). When investigating mucosal responses, it was observed that the addition of cAMP was helpful to the generation of serum IgA, fecal IgA, and IgG against the peptide epitope (FIGS. 5F-5H). As for PADRE, while IgG titers were comparable to those generated in PADRE-free groups, the addition of the T-cell epitope decreased detected IgA. Overall, the combination of C5a1-Coil29 with the adjuvant cAMP but without PADRE was chosen to move forward in following experiments, as the formulation maximized IgG and IgA levels in both serum and feces.

### Example 4

### Intranasal Immunization with C3a Epitopes on Coil29 Nanofibers

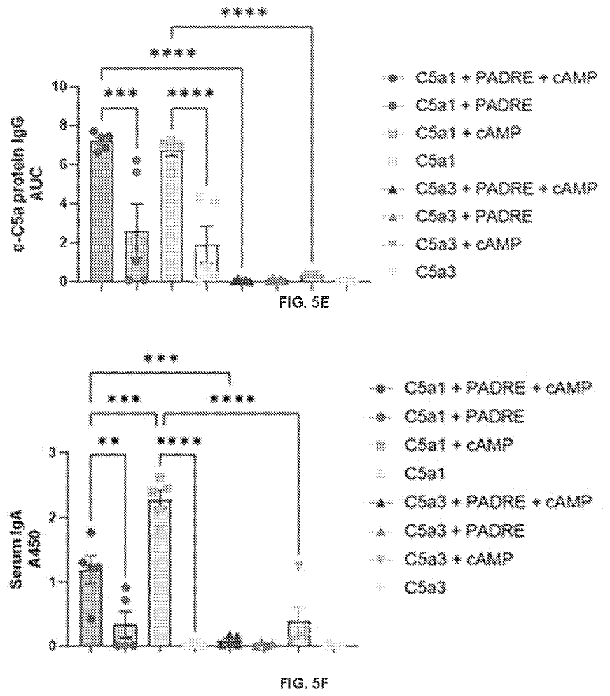

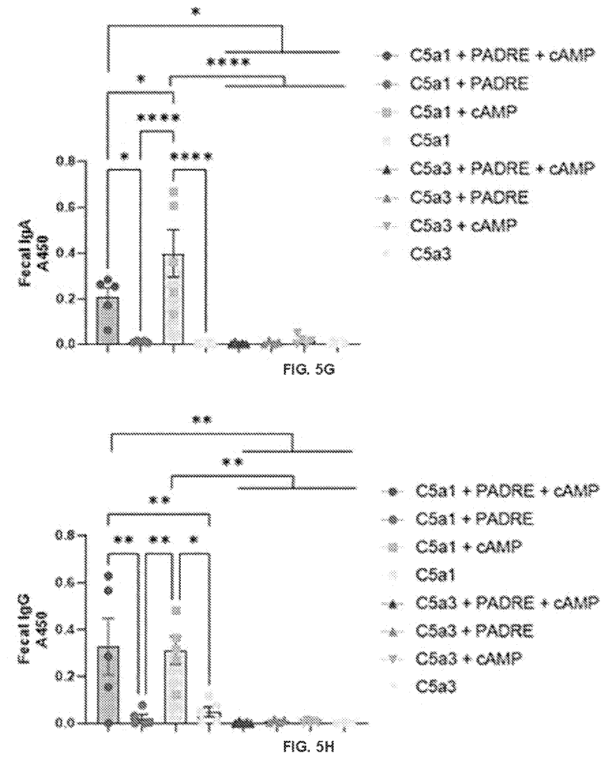

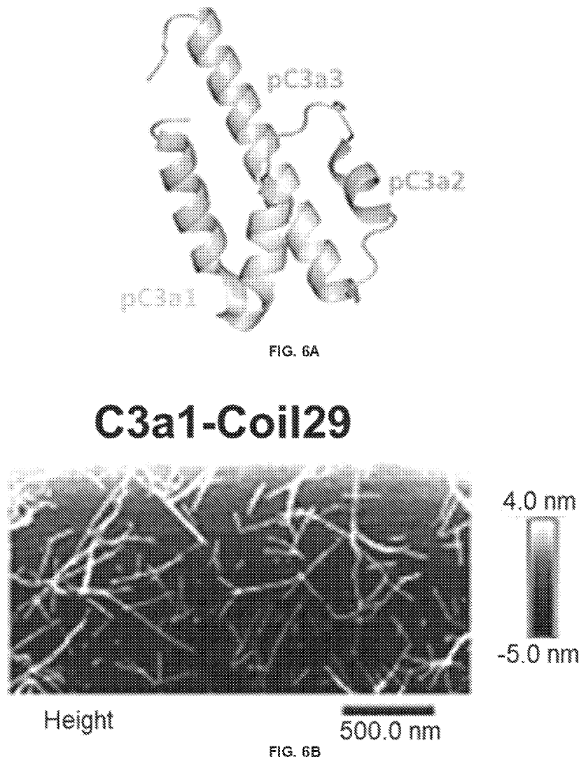

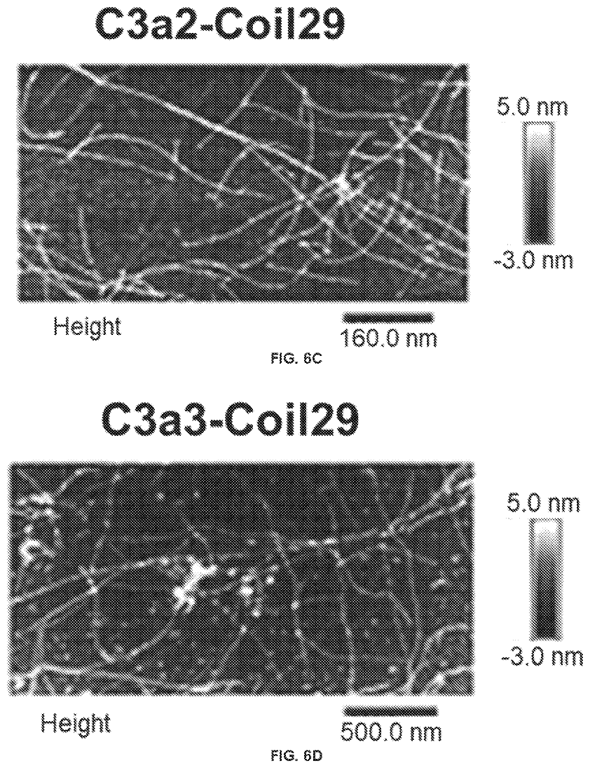

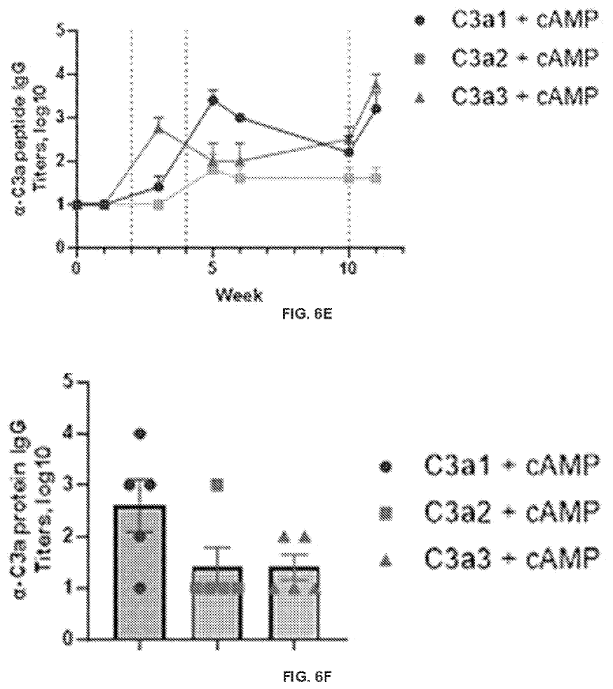

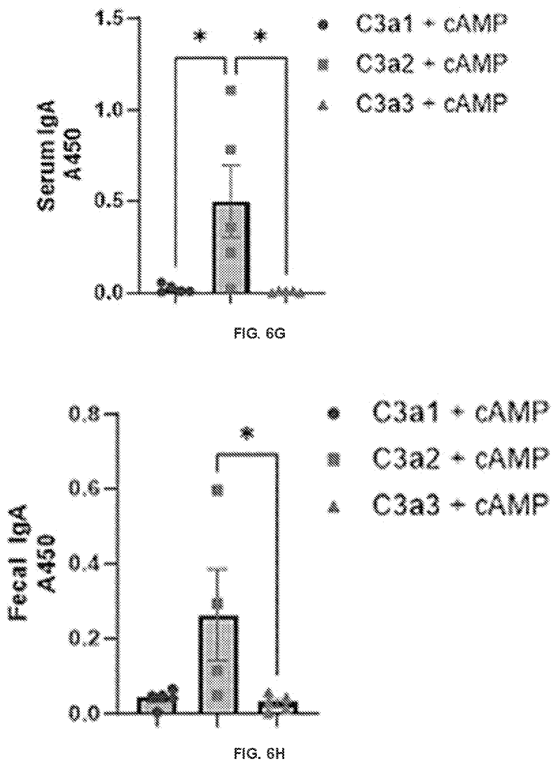

In addition to C5a, which has been previously explored as an active immunotherapy target, its sister molecule C3a was also investigated. Three novel C3a B-cell epitopes (pC3a1, pC3a2, and pC3a3) were predicted with the Kolaskar & Tongaonkar antigenicity test and synthesized on the N-terminus of Coil29 (FIG. 6A). Peptides were assembled into C3a1-Coil29, C3a2-Coil29, and C3a3-Coil29 nanofibers. Through AFM imaging, nanofiber formation and morphology were confirmed (FIGS. 6B-6D). Based on the results of immunization with C5a epitopes, all formulations included CAMP, but no PADRE. IgG titers in immunized animals were monitored for eleven weeks, and both C3a1-Coil29 and C3a2-Coil29 generated IgG against the corresponding peptide epitope (FIG. 6E). However, C3a1-Coil29 raised the highest IgG response against the entire C3a1 protein, with four out of five animals having significant titers, compared to one and two out of five for C3a2-Coil29 and C3a3-Coil29, respectively (FIG. 6F). C5a2-Coil29 was the only group to raise serum and fecal IgA, and all groups had detectable levels of fecal IgG (FIGS. 6G-I). T-cell responses against Coil29 and the C3a epitopes were assessed via ELISpot, and the results revealed an epitope-dependent biasing (FIGS. 6J-K). Both C3a1 and C3a3 had a Th1-biased response against Coil29, while C3a2 had a more balanced response, with no significant difference between IFNγ and IL-4 producing cells. C3a2 was the only peptide that showed capacity to be a T-cell epitope, with splenocytes responding to C3a2 stimulation with IL-4 production. Out of all these measures, anti-C3a protein IgG is the most important indicator of immunogenicity, therefore C3a1-Coil29 was chosen to move forward in this study. Importantly, C3a1 is not a T-cell epitope, which is helpful to avoid the formation of autoreactive T-cells.

### Example 5

### Design of Experiments Approach Enables Dual-Epitope Immunotherapy

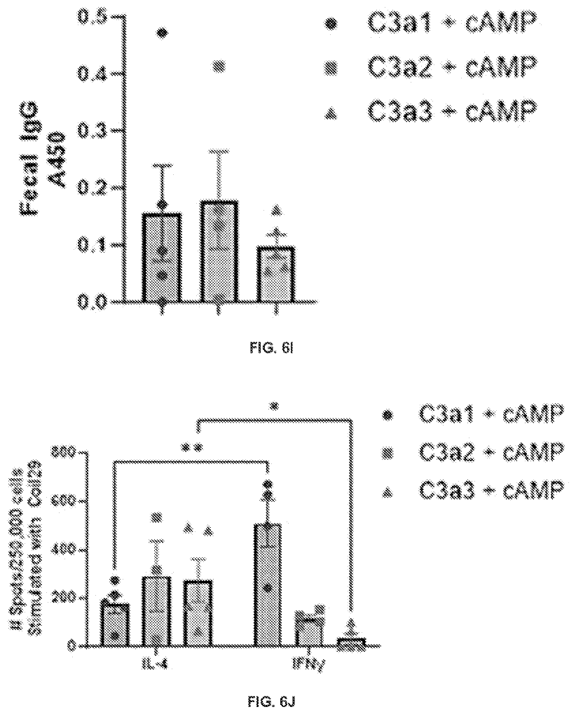

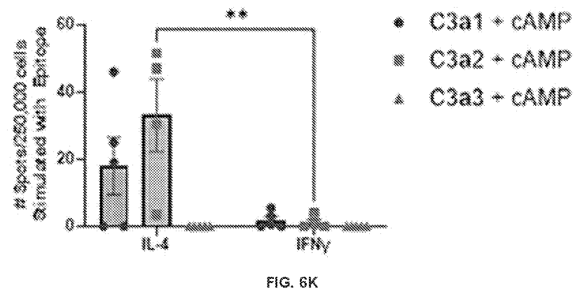

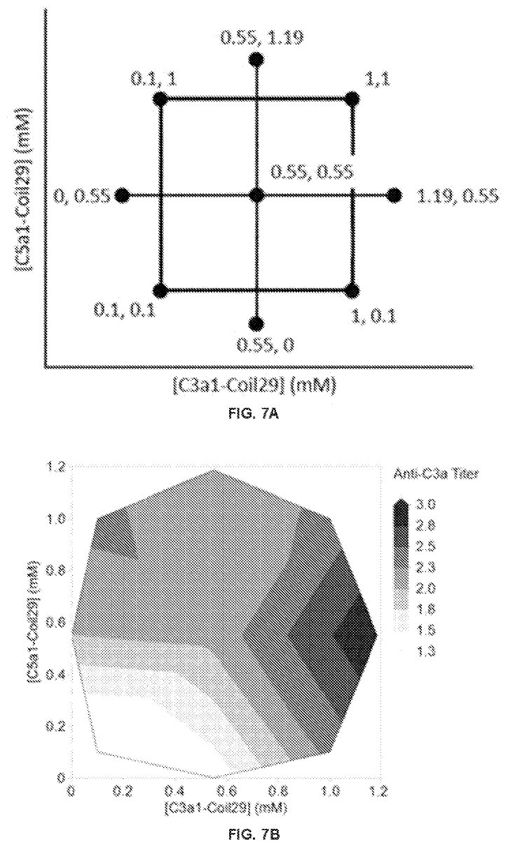

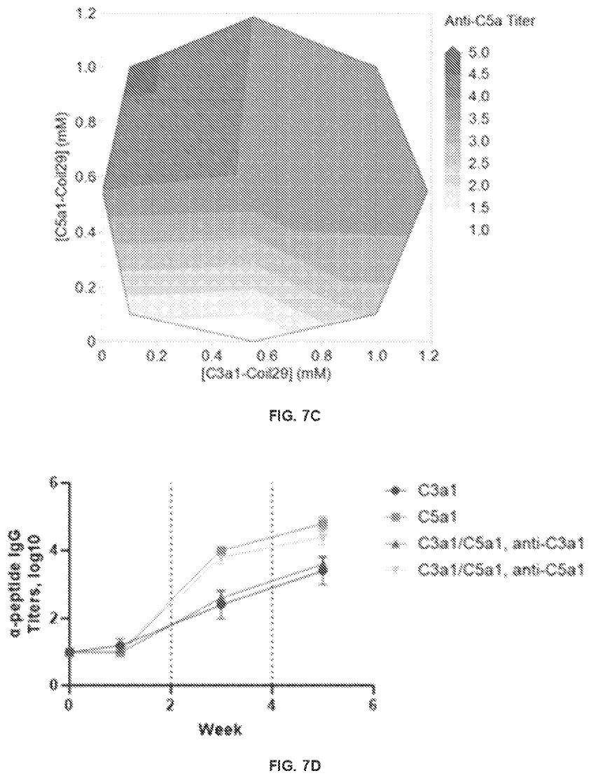

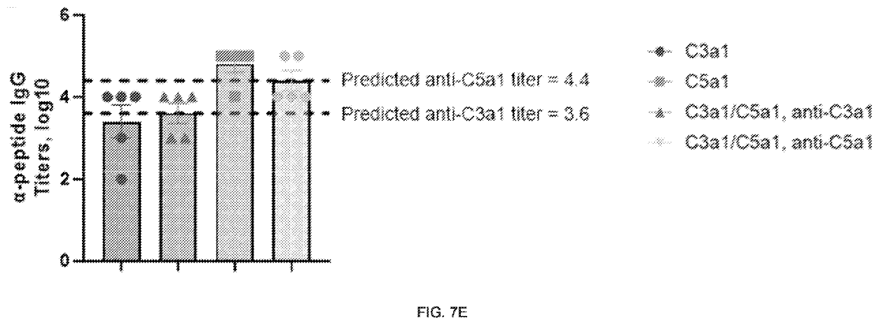

Once individual active immunotherapies were developed against C3a and C5a, a Design of Experiments (DoE) approach was utilized to identify the optimal combination of epitopes to maximize anti-C3a and anti-C5a titers with a single nanofiber formulation. The DoE is often used to predict the interaction between variables and maximize the overall response. Based on experimental groups that delineate a range of low, medium, and high concentrations of two variables, the model can predict a response surface of an outcome in a function of the chosen variables (FIG. 7A). Due to the modular nature of the Coil29 platform, epitopes were able to be titrated into nanofibers and their interplay with the DoE was able to be studied. Animals were immunized three times at weeks 0, 2, and 4 with the formulations delineated by the model, and at week five, serum IgG against both pC3a1 and pC5a1 peptides was measured. Experimental results were input in the DoE model to create a surface response curve of IgG titers as a function of epitope concentration. Anti-C3a1 titers were maximized at high concentrations of C3a1-Coil29 and intermediate concentrations of C5a1-Coil29 in the total nanofiber content (FIG. 7B), while anti-C5a1 titers were maximized at low concentrations of C3a1-Coil29 and high concentrations of C5a1-Coil29 (FIG. 7C). The lack of a linear relationship between epitope content and antibody titer revealed an inter-epitope dependency in raising antibodies against both complement anaphylatoxins with a single nanofiber formulation.

Predictive DoE results indicated that nanofibers containing 1.45 mM C3a1-Coil29 and 0.55 mM C5a1-Coil29 were ideal to maximize antibody response with dual-epitope immunotherapy. According to the model, this would result in an average anti-C3a1 IgG titer of 103.6 and anti-C5a1 IgG titer of 104.4, allowing for relatively high titers using a single formulation of coassembled epitopes. To validate antibody titer predictions, mice were immunized with individual epitopes or combination therapy at the predicted concentrations, and anti-peptide IgG levels were measured over time (FIG. 7D). Impressively, at week five, the average titers for C3a1/C5a1 immunized animals were exactly as predicted by the DoE model (FIG. 7E). This highlighted the power of the DoE to accurately predict complex responses to multi-epitope immunotherapies.

### Example 6

### Correlation Between Anti-C3a and Anti-C5a IgG Titers and Disease Outcome in LPS-Induced Sepsis Model

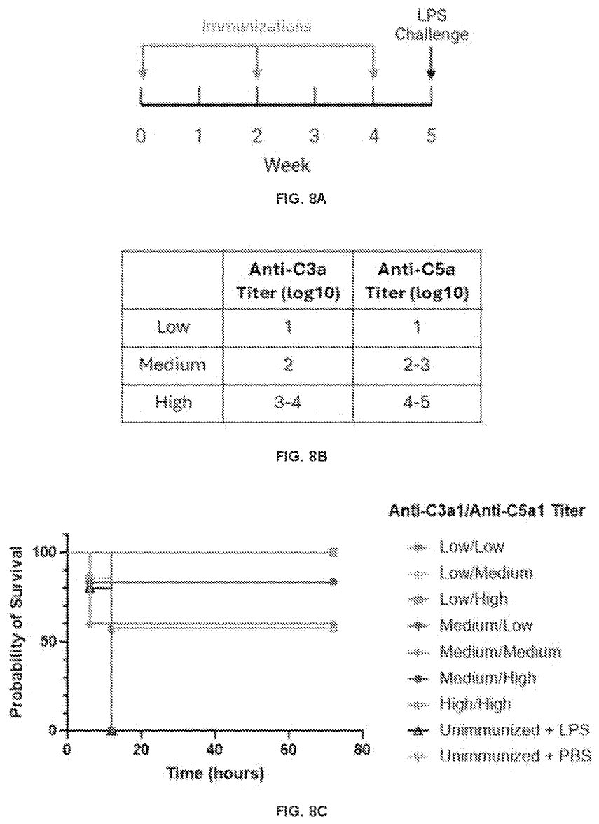

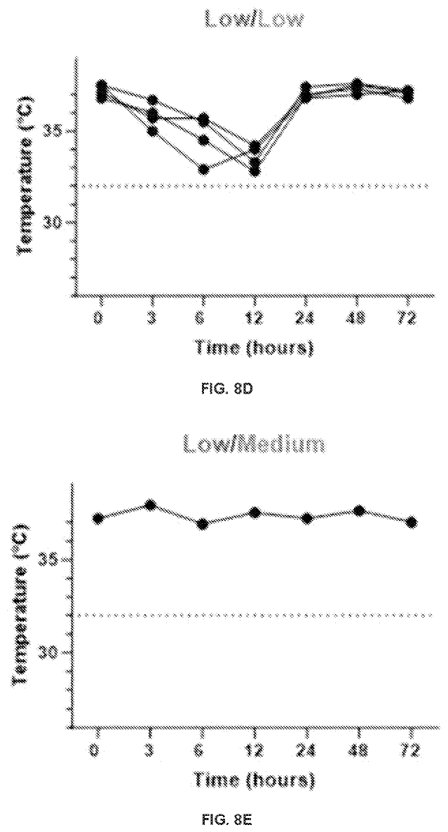

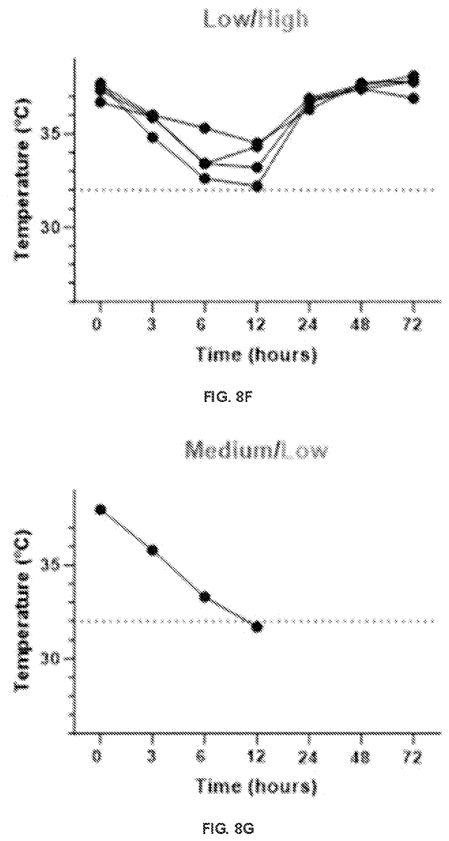

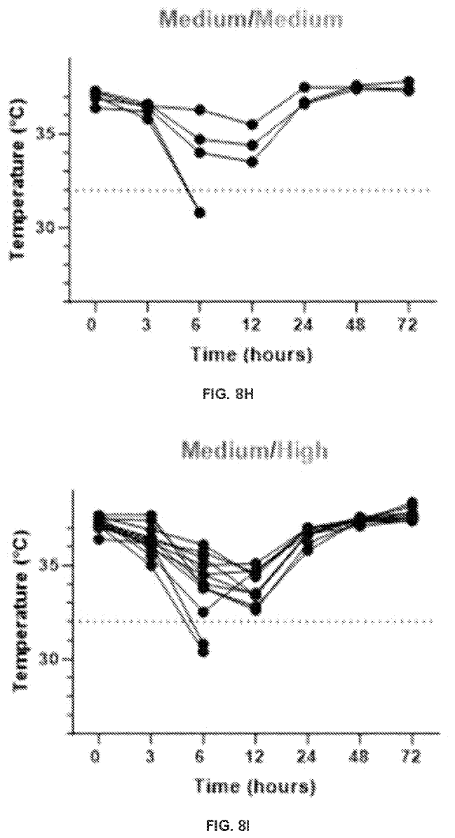

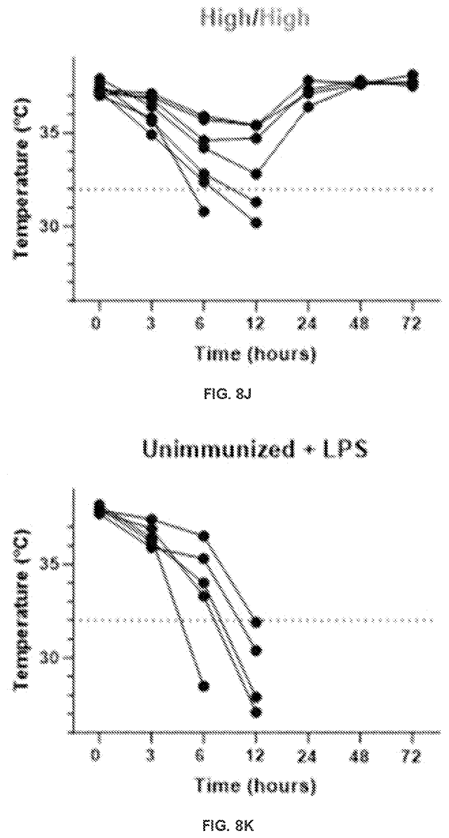

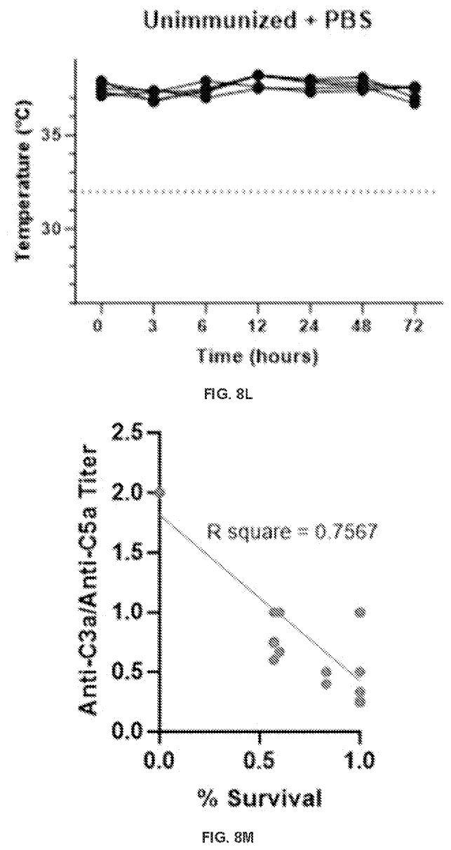

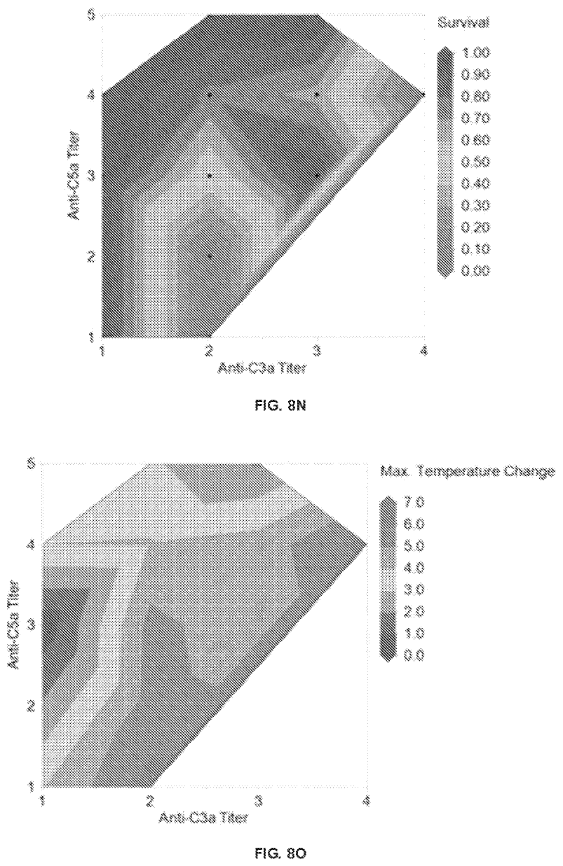

In addition to investigating how IgG titers against C3a and C5a are raised depending on epitope content, it was also studied how raising varying immune responses against complement anaphylatoxins interfered with a model of acute inflammation. To do so, mice were immunized at weeks 0, 2, and 4 with all formulations required for the DoE, and at week 5 intraperitoneal (IP) lipopolysaccharide (LPS) was administered to induce septic shock (FIG. 8A). After LPS injection, rectal temperature and survival were monitored for 72 hours.

Since there is an inherent variability with intranasal immunization, and the same formulation can induce varying levels of IgG in mice, animals were sorted depending on their anti-C3a and anti-C5a IgG titer. A titer of 101 was considered low for both molecules, medium titers were 102 for anti-C3a and 102-103 for anti-C5a, and high titers were >103 for anti-C3a and >104 for anti-C5a (FIG. 8B). The probability of survival for all observed combinations of titers was analyzed (FIG. 8C).

Temperature over time curves were plotted for each combination of low, medium, or high anti-C3a and anti-C5a titers (FIGS. 8D-L). A decreasing correlation between percent survival and anti-C3a/anti-C5a titer ratio was observed (FIG. 8M). In addition, summaries of survival (FIG. 8N) and maximum temperature change (FIG. 8O) data were plotted as a function of anti-C3a1 and anti-C5a1 titers to better visualize how generating immune responses against both complement anaphylatoxins interferes with disease outcome. It was observed that mice with low levels of anti-C3a, but medium or high levels of anti-C5a showed lower drops in rectal temperature as well as a 100% survival rate. Those with medium anti-C3a but high anti-C5a had a survival rate of 83%, and those with both medium or high anti-C3a titers had a 60% survival rate. Interestingly, immunized animals that had no detectable titers had a 100% survival rate, indicating the involvement of immune mechanisms other than antibodies to generate protection. This demonstrated that immune responses generated with the Coil29 platforms interfered with anaphylatoxin function in vivo. Animals with anti-C5a responses had increased survival, as shown previously with the Q11 platform (Hainline et al., Acta Biomaterialia, 2024, S1742-7061 (24) 00112-0). Those with dominating anti-C3a responses, however, had decreased survival and higher temperature changes, which matched previous descriptions of blockade of C3a in acute inflammation.

### Example 7

### Characterization of T-cell Responses and Mucosal Immunity Following a C3a1/C5a1-Coil29 Active Immunotherapy

While C3a and C5a seem to have opposing roles in acute inflammatory contexts, both anaphylatoxins have been shown to be pathogenic in chronic inflammatory diseases. Therefore, it is of interest to develop an active immunotherapy targeting C3a and C5a and further characterize its immune response. While a correlation between anti-C3a and anti-C5a titers with LPS outcomes has been observed, there might be other immune characteristics of this response at play.

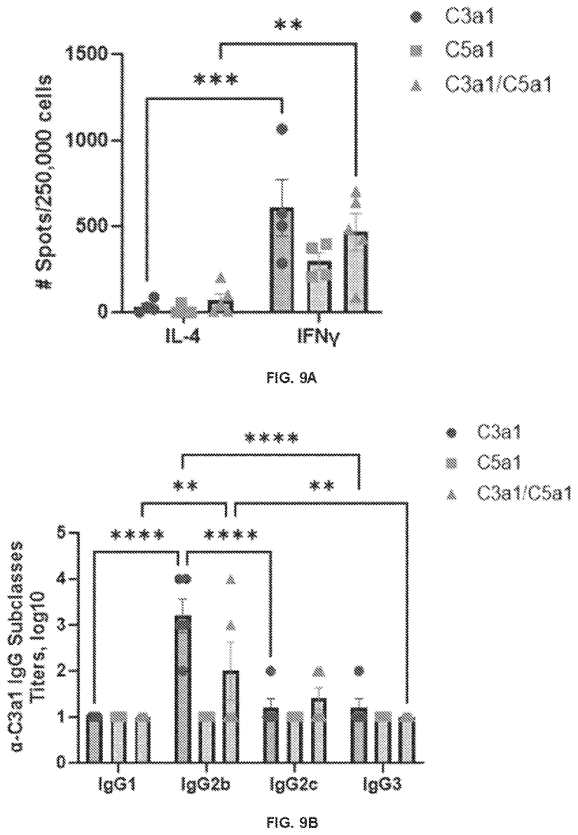

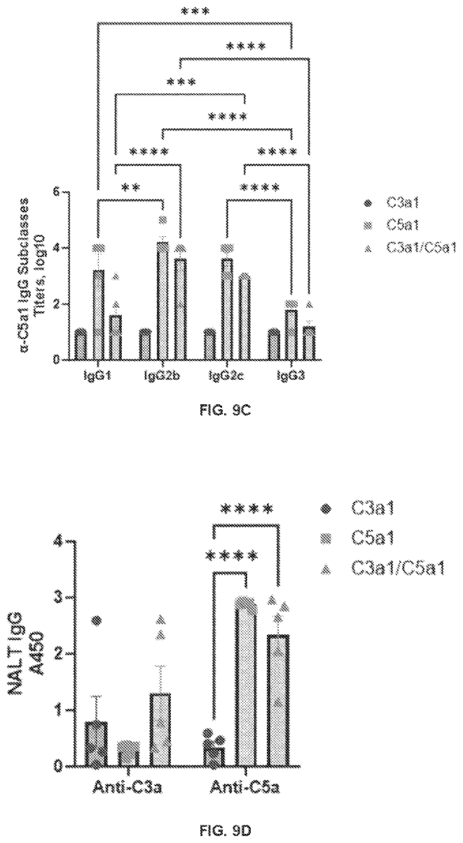

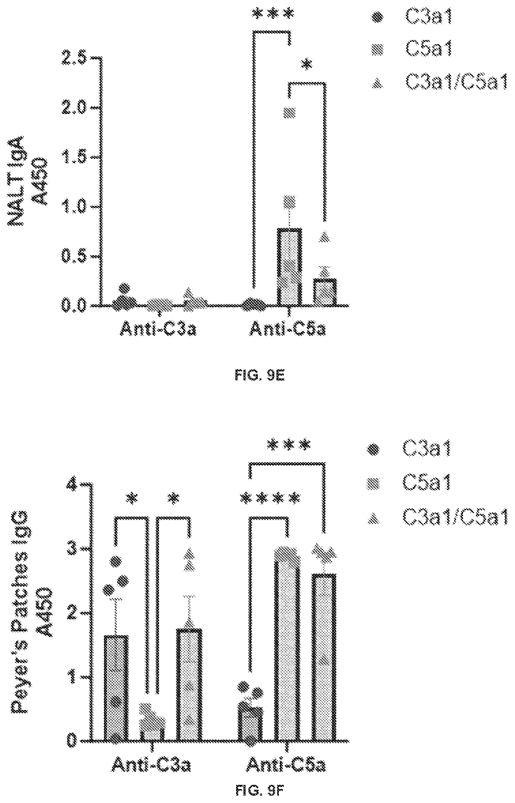

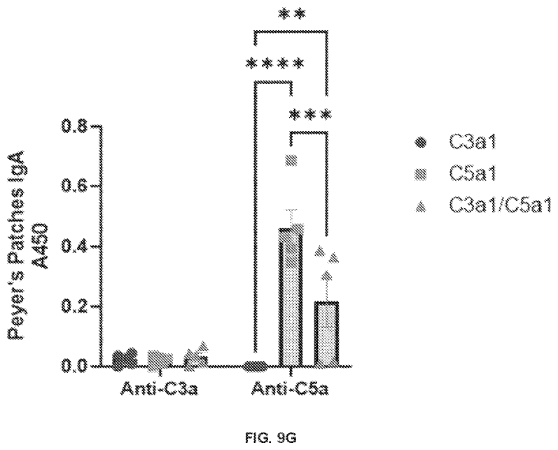

In order to characterize the T-cell response generated by the active immunotherapy, mice were immunized with the formulation that maximized anti-C3a and anti-C5a titers (1.45 mM C3a1-Coil29 and 0.55 mM C5a1-Coil29), as well as individual epitope formulations (1.45 mM C3a1-Coil29 or 0.55 mM C5a1-Coil29). Splenocytes from immunized mice were cultured, and IL-4 and IFNγ producing cells were counted after stimulation with unassembled Coil29 peptide via ELISpot (FIG. 9A). Additionally, anti-C3a1 (FIG. 9B) and anti-C5a1 (FIG. 9C) IgG subclasses were measured via ELISA. Together these results indicated a Th1-biased response for C3a1 and C3a1/C5a1 immunized animals, with significantly higher IFNγ-producing cells, as well as a higher proportion of IgG2b. In comparison, C5a1 immunized mice showed no significant difference between IL-4 and IFNγ-producing splenocytes and had an overall more balanced IgG response, indicating a less skewed T-cell response. This difference in T-cell bias correlated well with LPS challenge outcomes and might be a factor influencing animal survival and maximum temperature change.

Immune responses at mucosal tissues were also assessed. Anti-C5a1 NALT IgG (FIG. 9D) and IgA (FIG. 9E) were detected in C5a1 and C3a1/C5a1 immunized groups, anti-peptide IgG in Peyer's patches was detected in all groups (FIG. 9F), and anti-C5a1 IgA in C5a1 and C3a1/C5a1 groups (FIG. 9G). This is consistent with what was observed in the single epitope immunogenicity experiments, where C3a1-Coil29 was not able to raise IgA responses. The presence of epitope-specific antibodies in both NALT and Peyer's patches showed the ability of intranasal immunization to raise immune responses in the respiratory and gastrointestinal tract.

### Example 8

### DISCUSSION

Supramolecular assemblies have been successfully employed for the development of active immunotherapies against inflammatory self-molecules, such as cytokines and complement proteins. Here, the Coil29 platform was used to deliver C3a and C5a epitopes intranasally, which proved to be immunogenic with the addition of the STING adjuvant cAMP without any further material modifications. Intranasal immunization has many benefits over traditional subcutaneous or intramuscular immunotherapies, including the generation of IgA and immunity at mucosal tissues, and needle-free administration. The modular nature of the peptide nanofibers was leveraged in a DoE approach to study the interplay of the two complement anaphylatoxins in a model of acute inflammation by correlating immune responses against C3a and C5a and disease outcomes following LPS administration. Additionally, a dual-epitope platform was developed with both C3a and C5a epitopes as an active immunotherapy. The T-cell response was characterized, and immunity was generated at the NALT and Peyer's patches, showing that intranasal immunization can lead to immune responses at both the respiratory and gastrointestinal tracts.

Despite what was previously observed with subcutaneous administration of C5a epitopes, C5a1-Coil29 proved to be immunogenic when administered intranasally. Only one of the predicted C3a epitopes, C3a1-Coil29, was immunogenic against the C3a protein. However, unlike C5a1-Coil29, C3a1-Coil29 failed to induce IgA responses. The difference in T-cell profile between groups may also explain why C3a1-Coil29 did not induce IgA. IgA production has been likened to a Th2-bias and the IL-4 pathway. ELISpot data revealed a balanced response for C3a2-Coil29, the only group that generated IgA. Both C3a1 and C3a3 showed a Th1-biased response and did not generate IgA. This correlation between T-cell phenotype and IgA production may be further investigated. Nonetheless, C3a1-Coil29 and C5a1-Coil29 were selected as optimal epitopes to raise intranasal immune responses and were further investigated in a dual-epitope immunotherapy.

With a single nanofiber formulation, IgG titers were raised against C3a and C5a. The DoE results showed that antibody titer was dependent not only on the target peptide concentration, but also on the presence and concentration of the other epitope. When a formulation outside of the bounds of the original DoE was tested, anti-C3a1 and anti-C5a1 titers matched the predicted titers by the model. This highlighted the power of the DoE approach to maximize antibody responses when developing multi-epitope immunotherapies or vaccines, as immunodominance often plays a role and maximizing epitope content might not lead to the desired outcomes.

The results herein confirmed that C5a has a pro-inflammatory role in LPS-mediated sepsis, which is alleviated with anti-C5a1 immune responses. C3a, on the other hand, has an ambiguous role in inflammation, acting as an anti-inflammatory molecule during acute stages and as a pro-inflammatory mediator during chronic inflammation. Here, in a model of acute inflammation, it was observed that the presence of anti-C3a1 antibodies aggravated disease outcomes, which is in line with what has been previously observed by genetic blockade of C3a in mouse models of bacteremia. However, to the best of our knowledge, the interplay between C3a and C5a following an LPS challenge has not yet been studied. While targeting C5a alone was able to hinder disease progression, having immune responses against both anaphylatoxins was detrimental to animal survival and homeostasis. Survival rates negatively correlated to the ratio of anti-C3a1/anti-C5a1 titers, meaning that increasing anti-C5a responses was able to ameliorate the effect generated by anti-C3a responses when dominant. In light of previous discoveries of the individual roles of C3a and C5a, as well as novel findings about the interactions of both anaphylatoxins in acute inflammation, the data herein showed that the immune responses raised by Coil29 active immunotherapies are able to interfere with C3a and C5a activity in vivo.

Interestingly, immunized animals with no anti-C3a or anti-C5a titers showed a 100% survival rate. These animals received nanofibers containing either 0.1 mM C3a1-Coil29 and 0.1 mM C5a1-Coil29 or either 0.55 mM C3a1-Coil29 and 0 mM C5a1-Coil29.

The T-cell bias of immune responses may be considered in addition to antibodies as a contributing factor to LPS challenge outcomes. Previously, it was shown that a Th-1 biased immune response was detrimental in LPS-induced sepsis after an active immunotherapy against TNF. Here, C5a1 immunized mice showed a less skewed T-cell response than their C3a1 and C3a1/C5a1 immunized counterparts, which corresponded to more positive outcomes following LPS administration. It is possible that, in addition to antibody-mediated blockade of anaphylatoxins, the difference in Th-bias of the immune response was playing a role in the immunological response after septic shock.

Mucosal immune responses were observed as a result of the active immunotherapy. Epitope-specific IgG and IgA were detected in the NALT following intranasal immunization. As both nanofibers and adjuvant are rapidly delivered to the tissue, NALT antibody production was expected. Interestingly, the platform also raised IgG and IgA in feces and Peyer's patches. This crosstalk between mucosae has been extensively observed, and it allows for immunization at one site while promoting immunity at distal mucosal sites, i.e., intranasal immunization can lead to responses in the gastrointestinal tract. Therefore, intranasal active immunotherapies against complement anaphylatoxins might have therapeutic effects in chronic inflammatory conditions that affect both respiratory and gastrointestinal mucosal tissues. Unlike in LPS-induced sepsis, the pathology of these diseases may be aggravated by the presence of both C3a and C5a, so the formulation herein developed could have beneficial therapeutic effects.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. An anaphylatoxin conjugate peptide comprising: (i) a self-assembling peptide comprising a polypeptide having the amino acid sequence of bXXXb (SEQ ID NO: 1, wherein X is independently any amino acid and b is independently any positively charged amino acid), QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and (ii) at least one anaphylatoxin epitope conjugated to a terminus of the self-assembling peptide, wherein the anaphylatoxin epitope comprises a C3a anaphylatoxin or a C5a anaphylatoxin, or a combination thereof.

Clause 2. The anaphylatoxin conjugate peptide of clause 1, wherein the anaphylatoxin epitope comprises a C3a anaphylatoxin.

Clause 3. The anaphylatoxin conjugate peptide of clause 1 or 2, wherein the C3a anaphylatoxin epitope comprises C3a1, C3a2, or C3a3, or a combination thereof.

Clause 4. The anaphylatoxin conjugate peptide of clause 1, wherein the anaphylatoxin epitope comprises a C5a anaphylatoxin.

Clause 5. The anaphylatoxin conjugate peptide of clause 1 or 4, wherein the C5a anaphylatoxin epitope comprises C5a1 or C5a3, or a combination thereof.

Clause 6. The anaphylatoxin conjugate peptide of any one of clauses 1-5, wherein the anaphylatoxin epitope comprises C3a1, C3a2, C3a3, C5a1, or C5a3, or a combination thereof.

Clause 7. The anaphylatoxin conjugate peptide of any one of clauses 1-6, wherein the anaphylatoxin epitope comprises the amino acid sequence of SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, or SEQ ID NO: 114, or a combination thereof.

Clause 8. The anaphylatoxin conjugate peptide of any one of clauses 1-7, wherein each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

Clause 9. The anaphylatoxin conjugate peptide of any one of clauses 1-7, wherein each self-assembling peptide forms an alpha-helix and comprises a polypeptide having an amino acid sequence of bXXXb (SEQ ID NO: 1), wherein X is independently any amino acid and b is independently any positively charged amino acid.

Clause 10. The anaphylatoxin conjugate peptide according to clause 8 or 9, wherein the self-assembling peptide comprises the sequence QQKFQFQFEQQ (SEQ ID NO: 12) or Ac-QQKFQFQFEQQ-NH₂ (SEQ ID NO: 13) or bXXXb (SEQ ID NO: 1, wherein X is independently any amino acid and b is independently any positively charged amino acid).

Clause 11. The anaphylatoxin conjugate peptide of any one of clauses 1-7 and 9-10, wherein b is independently selected from Arg and Lys.

Clause 12. The anaphylatoxin conjugate peptide of any one of clauses 1-7 and 9-11, wherein bXXXb (SEQ ID NO: 1) is RAYAR (SEQ ID NO: 2) or KAYAK (SEQ ID NO: 3).

Clause 13. The anaphylatoxin conjugate peptide of any one of clauses 1-7 and 9-12, wherein the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20.

Clause 14. The anaphylatoxin conjugate peptide of clause 13, wherein the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 9), or Ac-QAKILEADAEILKAYAKILEAHAEILKAQ-NH₂ (SEQ ID NO: 10), or Ac-ADAEILRAYARILEAHAEILRAQ-NH₂ (SEQ ID NO: 11), or QAEILRAYARILEAQ (SEQ ID NO: 101), or QAEILRAYARILEAHAEILKAQ (SEQ ID NO: 102), or QAEILRAYARILEADAKILEAHAEILKAQ (SEQ ID NO: 103), or QAEILRAYARILEADAEILKAQAKILEAHAEILKAQ (SEQ ID NO: 104).

Clause 15. The anaphylatoxin conjugate peptide of any one of clauses 1-14, wherein the self-assembling peptide forms a beta sheet and the at least one anaphylatoxin epitope is attached to the C-terminus or the N-terminus of the self-assembling peptide or a combination thereof, or wherein the self-assembling peptide forms an alpha-helix and the at least one anaphylatoxin epitope is attached to the N-terminus of the self-assembling peptide.

Clause 16. The anaphylatoxin conjugate peptide of any one of clauses 1-15, wherein 1 to 10 anaphylatoxin epitopes are attached to the C-terminus or the N-terminus of the self-assembling peptide.

Clause 17. The anaphylatoxin conjugate peptide of any one of clauses 1-16, wherein the self-assembling peptide forms a beta sheet and the anaphylatoxin conjugate peptide further comprises: (iii) a PEG molecule or a PAS peptide conjugated to the self-assembling peptide.

Clause 18. The anaphylatoxin conjugate peptide of clause 17, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto.

Clause 19. The anaphylatoxin conjugate peptide of clause 17, wherein the PEG molecule comprises PEG-2000.

Clause 20. The anaphylatoxin conjugate of any one of clauses 17-19, wherein the PEG molecule or the PAS peptide is conjugated to the self-assembling peptide at the same or the opposite terminus from wherein the anaphylatoxin epitope is attached.

Clause 21. The anaphylatoxin conjugate peptide of any one of clauses 1-20, further comprising: (iv) at least one linker.

Clause 22. The anaphylatoxin conjugate peptide of clause 21, wherein the at least one linker is between the at least one anaphylatoxin epitope and the self-assembling peptide.

Clause 23. The anaphylatoxin conjugate peptide of clause 21, wherein the at least one linker comprises a first linker between the at least one anaphylatoxin epitope and the self-assembling peptide, and a second linker between the PEG molecule or the PAS peptide and the self-assembling peptide.

Clause 24. The anaphylatoxin conjugate peptide of any one of clauses 21-23, wherein the at least one linker comprises SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) 8), SEQ ID NO: 92 ((G4S) 3), SEQ ID NO: 93 (KSGSG), SEQ ID NO: 94 (KKSGSG), SEQ ID NO: 95 (EAAAK) 2, or SEQ ID NO: 96 (GGAAY).

Clause 25. The anaphylatoxin conjugate peptide of any one of clauses 1-7, 9-16, 21-22, and 24, wherein the anaphylatoxin conjugate peptide comprises C3a1-Coil29, C3a2-Coil29, C3a3-Coil29, C5a1-Coil29, or C5a3-Coil29.

Clause 26. The anaphylatoxin conjugate peptide of any one of clauses 1-7, 9-16, 21-22, and 24-25, wherein the anaphylatoxin conjugate peptide comprises SEQ ID NO: 115, SEQ ID NO: 116, SEQ ID NO: 117, SEQ ID NO: 118, or SEQ ID NO: 119, or a combination thereof.

Clause 27. A nanofiber comprising a plurality of the anaphylatoxin conjugate peptide of any one of clauses 1-26, wherein the anaphylatoxin conjugate peptides self-assemble into the nanofiber.

Clause 28. A nanofiber comprising: (i) at least one anaphylatoxin conjugate peptide of any one of clauses 1-20; and (ii) at least one T-cell epitope-conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and at least one T-cell epitope conjugated to a terminus of the self-assembling peptide, wherein the at least one T-cell epitope is selected from PADRE and VAC, and wherein PADRE comprises a polypeptide having the amino acid sequence of aKXVAAWTLKAa (SEQ ID NO: 99, wherein “X” comprises cyclohexylalanine and “a” comprises D-alanine), and wherein VAC comprises a polypeptide having the amino acid sequence of QLVFNSISARALKAY (SEQ ID NO: 100).

Clause 29. The nanofiber of clause 28, wherein the cyclohexylalanine comprises D-alanine.

Clause 30. The nanofiber of clause 28 or 29, wherein the T-cell epitope-conjugate peptide further comprises a linker between the T-cell epitope and the self-assembling peptide.

Clause 31. The nanofiber of clause 30, wherein the linker comprises an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) 8), SEQ ID NO: 92 ((G4S) 3), SEQ ID NO: 93 (KSGSG), SEQ ID NO: 94 (KKSGSG), SEQ ID NO: 95 (EAAAK) 2, and SEQ ID NO: 96 (GGAAY).

Clause 32. The nanofiber of any one of clauses 27-31, further comprising: (iii-a) at least one PEG conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), and at least one PEG molecule conjugated to a terminus of the self-assembling peptide; or (iii-b) at least one PAS conjugate peptide comprising: a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), and at least one PAS peptide conjugated to a terminus of the self-assembling peptide, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto.

Clause 33. The nanofiber of any one of clauses 27-32, further comprising: (iv) a plain self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

Clause 34. The nanofiber of any one of clauses 27-33, wherein the nanofiber comprises a combination of 10 to 10,000, or 100 to 10,000 anaphylatoxin conjugate peptides.

Clause 35. The nanofiber of any one of clauses 27-34, wherein the nanofiber comprises a plurality of the same or different anaphylatoxin epitopes.

Clause 36. The nanofiber of any one of clauses 33-35, wherein the nanofiber comprises a combination of 10 to 10,000, or 100 to 10,000 peptides comprising anaphylatoxin conjugate peptides, T-cell epitope-conjugate peptides, and plain self-assembling peptides.

Clause 37. The nanofiber of any one of clauses 32-36, wherein the nanofiber comprises a combination of 10 to 10,000, or 100 to 10,000 peptides comprising anaphylatoxin conjugate peptides, T-cell epitope-conjugate peptides, PAS conjugate peptides or PEG conjugate peptides, and plain self-assembling peptides.

Clause 38. The nanofiber of any one of clauses 33-37, wherein the nanofiber comprises a combination of 10 to 10,000, or 100 to 10,000 peptides comprising anaphylatoxin conjugate peptides, PAS conjugate peptides or PEG conjugate peptides, and plain self-assembling peptides.

Clause 39. The nanofiber of any one of clauses 27-38, wherein at least about 1%, 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or 97.5% of the peptides in the nanofiber are anaphylatoxin conjugate peptides.

Clause 40. The nanofiber of any one of clauses 28-39, wherein at least about 1%, 2%, 3%, 4%, 5%, 6%, 7%, 8%, 9%, 10%, 11%, 12%, 13%, 14%, or 15% of the peptides in the nanofiber are T-cell epitope-conjugate peptides.

Clause 41. The nanofiber of any one of clauses 28-40, wherein the anaphylatoxin conjugate peptide and the T-cell epitope-conjugate peptide are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

Clause 42. The nanofiber of any one of clauses 27-41, wherein the self-assembling peptide forms a fibril including beta-sheet structures or a fibril having a coiled coil structure.

Clause 43. The nanofiber of any one of clauses 27-41, wherein the self-assembling peptide forms a fibril having a structure of a helical filament formed around a central axis.

Clause 44. The nanofiber of clause 43, wherein the N-terminus of each self-assembling peptide is positioned at the exterior of the helical filament.

Clause 45. The nanofiber of any one of clauses 27-44, wherein the anaphylatoxin epitopes are exposed on the exterior surface of the nanofiber.

Clause 46. The nanofiber of any one of clauses 27-45, wherein the nanofiber is about 5-30 nm in width.

Clause 47. The nanofiber of any one of clauses 27-46, wherein the nanofiber is about 100 nm to 1 μm, 100 nm to 2 μm, 100 nm to 3 μm, 100 nm to 4 μm, or 100 nm to 5 μm in length.

Clause 48. A pharmaceutical composition comprising: (a) the anaphylatoxin conjugate peptide of any one of clauses 1-26 or the nanofiber of any one of clauses 27-47; and (b) a pharmaceutically acceptable carrier, diluent, and/or excipient.

Clause 49. The pharmaceutical composition of clause 48, further comprising: (c) an adjuvant selected from cyclic-di-AMP, CpG, cyclic GMP-AMP (cGAMP), cholera toxin B subunit (CTB), retinoic acid, heat labile toxin B subunit, alum, MF59, 3M-052, iscomatrix, squalene-based adjuvants, AS01, AS03, or AS04, or a combination thereof.

Clause 50. The pharmaceutical composition of clause 48 or 49, formulated into a tablet.

Clause 51. The pharmaceutical composition of clause 50, wherein the tablet is a dissolving tablet.

Clause 52. A method of treating an inflammatory condition, the method comprising administering to a subject a therapeutically effective amount of the anaphylatoxin conjugate peptide of any one of clauses 1-26, or the nanofiber of any one of clauses 27-47, or the pharmaceutical composition of any one of clauses 48-51.

Clause 53. The method of clause 52, wherein the inflammatory condition comprises inflammatory bowel disease (IBD), sepsis, ulcerative colitis, Crohn's disease, rheumatoid arthritis, psoriasis, cirrhosis, atherosclerosis, or cardiovascular inflammation, lupus, multiple sclerosis, Alzheimer's disease, pulmonary fibrosis, immunosenescence, or a combination thereof.

Clause 54. A method of reducing inflammation in a subject, the method comprising administering to the subject a therapeutically effective amount of the anaphylatoxin conjugate peptide of any one of clauses 1-26, or the nanofiber of any one of clauses 27-47, or the pharmaceutical composition of any one of clauses 48-51.

Clause 55. The method of any one of clauses 52-54, wherein the inflammation is chronic or acute.

Clause 56. A method of treating sepsis, the method comprising administering to a subject a therapeutically effective amount of the anaphylatoxin conjugate peptide of any one of clauses 1-26, or the nanofiber of any one of clauses 27-47, or the pharmaceutical composition of any one of clauses 48-51.

Clause 57. The method of any one of clauses 52-56, wherein the anaphylatoxin conjugate peptide or the nanofiber or the pharmaceutical composition is administered sublingually.

Clause 58. The method of any one of clauses 52-57, wherein the subject forms an antibody to the anaphylatoxin epitope.

Clause 59. The method of clause 58, wherein the antibody comprises IgG and/or IgA.

What is claimed is:

1. An anaphylatoxin conjugate peptide comprising:
(i) a self-assembling peptide comprising a polypeptide having the amino acid sequence of bXXXb (SEQ ID NO: 1, wherein X is independently any amino acid and b is independently any positively charged amino acid), QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and
(ii) at least one anaphylatoxin epitope conjugated to a terminus of the self-assembling peptide, wherein the anaphylatoxin epitope comprises a C3a anaphylatoxin or a C5a anaphylatoxin, or a combination thereof.

2. The anaphylatoxin conjugate peptide of claim 1, wherein the anaphylatoxin epitope comprises a C3a anaphylatoxin.

3. The anaphylatoxin conjugate peptide of claim 1 or 2, wherein the C3a anaphylatoxin epitope comprises C3a1, C3a2, or C3a3, or a combination thereof.

4. The anaphylatoxin conjugate peptide of claim 1, wherein the anaphylatoxin epitope comprises a C5a anaphylatoxin.

5. The anaphylatoxin conjugate peptide of claim 1 or 4, wherein the C5a anaphylatoxin epitope comprises C5a1 or C5a3, or a combination thereof.

6. The anaphylatoxin conjugate peptide of any one of claims 1-5, wherein the anaphylatoxin epitope comprises C3a1, C3a2, C3a3, C5a1, or C5a3, or a combination thereof.

7. The anaphylatoxin conjugate peptide of any one of claims 1-6, wherein the anaphylatoxin epitope comprises the amino acid sequence of SEQ ID NO: 105, SEQ ID NO: 106, SEQ ID NO: 107, SEQ ID NO: 108, SEQ ID NO: 109, SEQ ID NO: 110, SEQ ID NO: 111, SEQ ID NO: 112, SEQ ID NO: 113, or SEQ ID NO: 114, or a combination thereof.

8. The anaphylatoxin conjugate peptide of any one of claims 1-7, wherein each self-assembling peptide forms a beta sheet and comprises a polypeptide having an amino acid sequence selected from QQKFQFQFEQQ (SEQ ID NO: 12), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

9. The anaphylatoxin conjugate peptide of any one of claims 1-7, wherein each self-assembling peptide forms an alpha-helix and comprises a polypeptide having an amino acid sequence of bXXXb (SEQ ID NO: 1), wherein X is independently any amino acid and b is independently any positively charged amino acid.

10. The anaphylatoxin conjugate peptide according to claim 8 or 9, wherein the self-assembling peptide comprises the sequence QQKFQFQFEQQ (SEQ ID NO: 12) or Ac-QQKFQFQFEQQ-NH₂ (SEQ ID NO: 13) or bXXXb (SEQ ID NO: 1, wherein X is independently any amino acid and b is independently any positively charged amino acid).

11. The anaphylatoxin conjugate peptide of any one of claims 1-7 and 9-10, wherein b is independently selected from Arg and Lys.

12. The anaphylatoxin conjugate peptide of any one of claims 1-7 and 9-11, wherein bXXXb (SEQ ID NO: 1) is RAYAR (SEQ ID NO: 2) or KAYAK (SEQ ID NO: 3).

13. The anaphylatoxin conjugate peptide of any one of claims 1-7 and 9-12, wherein the self-assembling peptide comprises an amino acid sequence of ZₙbXXXbZₘ (SEQ ID NO: 5), wherein b is independently any positively charged amino acid, Z is independently any amino acid, X is independently any amino acid, n is an integer from 0 to 20, and m is an integer from 0 to 20.

14. The anaphylatoxin conjugate peptide of claim 13, wherein the self-assembling peptide comprises an amino acid sequence selected from QARILEADAEILRAYARILEAHAEILRAQ (Coil29, SEQ ID NO: 6), or QAKILEADAEILKAYAKILEAHAEILKAQ (SEQ ID NO: 7), or ADAEILRAYARILEAHAEILRAQ (SEQ ID NO: 8), or Ac-QARILEADAEILRAYARILEAHAEILRAQ-NH₂ (SEQ ID NO: 9), or Ac-QAKILEADAEILKAYAKILEAHAEILKAQ-NH₂ (SEQ ID NO: 10), or Ac-ADAEILRAYARILEAHAEILRAQ-NH₂ (SEQ ID NO: 11), or QAEILRAYARILEAQ (SEQ ID NO: 101), or QAEILRAYARILEAHAEILKAQ (SEQ ID NO: 102), or QAEILRAYARILEADAKILEAHAEILKAQ (SEQ ID NO: 103), or QAEILRAYARILEADAEILKAQAKILEAHAEILKAQ (SEQ ID NO: 104).

15. The anaphylatoxin conjugate peptide of any one of claims 1-14, wherein the self-assembling peptide forms a beta sheet and the at least one anaphylatoxin epitope is attached to the C-terminus or the N-terminus of the self-assembling peptide or a combination thereof,
or wherein the self-assembling peptide forms an alpha-helix and the at least one anaphylatoxin epitope is attached to the N-terminus of the self-assembling peptide.

16. The anaphylatoxin conjugate peptide of any one of claims 1-15, wherein 1 to 10 anaphylatoxin epitopes are attached to the C-terminus or the N-terminus of the self-assembling peptide.

17. The anaphylatoxin conjugate peptide of any one of claims 1-16, wherein the self-assembling peptide forms a beta sheet and the anaphylatoxin conjugate peptide further comprises:
(iii) a PEG molecule or a PAS peptide conjugated to the self-assembling peptide.

18. The anaphylatoxin conjugate peptide of claim 17, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto.

19. The anaphylatoxin conjugate peptide of claim 17, wherein the PEG molecule comprises PEG-2000.

20. The anaphylatoxin conjugate of any one of claims 17-19, wherein the PEG molecule or the PAS peptide is conjugated to the self-assembling peptide at the same or the opposite terminus from wherein the anaphylatoxin epitope is attached.

21. The anaphylatoxin conjugate peptide of any one of claims 1-20, further comprising:
(iv) at least one linker.

22. The anaphylatoxin conjugate peptide of claim 21, wherein the at least one linker is between the at least one anaphylatoxin epitope and the self-assembling peptide.

23. The anaphylatoxin conjugate peptide of claim 21, wherein the at least one linker comprises a first linker between the at least one anaphylatoxin epitope and the self-assembling peptide, and a second linker between the PEG molecule or the PAS peptide and the self-assembling peptide.

24. The anaphylatoxin conjugate peptide of any one of claims 21-23, wherein the at least one linker comprises SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) 8), SEQ ID NO: 92 ((G4S) 3), SEQ ID NO: 93 (KSGSG), SEQ ID NO: 94 SGSG), SEQ ID NO: 95 (EAAAK) 2, or SEQ ID NO: 96 (GGAAY).

25. The anaphylatoxin conjugate peptide of any one of claims 1-7, 9-16, 21-22, and 24, wherein the anaphylatoxin conjugate peptide comprises C3a1-Coil29, C3a2-Coil29, C3a3-Coil29, C5a1-Coil29, or C5a3-Coil29.

26. The anaphylatoxin conjugate peptide of any one of claims 1-7, 9-16, 21-22, and 24-25, wherein the anaphylatoxin conjugate peptide comprises SEQ ID NO: 115, SEQ ID NO: 116, SEQ ID NO: 117, SEQ ID NO: 118, or SEQ ID NO: 119, or a combination thereof.

27. A nanofiber comprising a plurality of the anaphylatoxin conjugate peptide of any one of claims 1-26, wherein the anaphylatoxin conjugate peptides self-assemble into the nanofiber.

28. A nanofiber comprising:
(i) at least one anaphylatoxin conjugate peptide of any one of claims 1-20; and
(ii) at least one T-cell epitope-conjugate peptide comprising:
a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn); and
at least one T-cell epitope conjugated to a terminus of the self-assembling peptide, wherein the at least one T-cell epitope is selected from PADRE and VAC, and wherein PADRE comprises a polypeptide having the amino acid sequence of aKXVAAWTLKAa (SEQ ID NO: 99, wherein “X” comprises cyclohexylalanine and “a” comprises D-alanine), and wherein VAC comprises a polypeptide having the amino acid sequence of QLVFNSISARALKAY (SEQ ID NO: 100).

29. The nanofiber of claim 28, wherein the cyclohexylalanine comprises D-alanine.

30. The nanofiber of claim 28 or 29, wherein the T-cell epitope-conjugate peptide further comprises a linker between the T-cell epitope and the self-assembling peptide.

31. The nanofiber of claim 30, wherein the linker comprises an amino acid sequence selected from SEQ ID NO: 83 (SGSG), SEQ ID NO: 84 ((Ser-Gly)₂), SEQ ID NO: 85 (CCCCSGSG), SEQ ID NO: 86 (Gₙ wherein n is an integer from 1 to 10), SEQ ID NO: 87 (GSGS), SEQ ID NO: 88 (SSSS), SEQ ID NO: 89 (GGGS), SEQ ID NO: 90 (GGC), SEQ ID NO: 91 ((GGC) 8), SEQ ID NO: 92 ((G4S) 3), SEQ ID NO: 93 (KSGSG), SEQ ID NO: 94 SGSG), SEQ ID NO: 95 (EAAAK) 2, and SEQ ID NO: 96 (GGAAY).

32. The nanofiber of any one of claims 27-31, further comprising:
(iii-a) at least one PEG conjugate peptide comprising:
a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), and
at least one PEG molecule conjugated to a terminus of the self-assembling peptide; or (iii-b) at least one PAS conjugate peptide comprising:
a self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn), and
at least one PAS peptide conjugated to a terminus of the self-assembling peptide, wherein the PAS peptide comprises a sequence of Pro-Ala-Ser or comprises the sequence of ASPAAPAPASPAAPAPSAPA (SEQ ID NO: 97), or a peptide having at least 80%, 85%, 90%, or 95% identity thereto.

33. The nanofiber of any one of claims 27-32, further comprising:
(iv) a plain self-assembling peptide comprising a polypeptide having the amino acid sequence of QQKFQFQFEQQ (SEQ ID NO: 12), bXXXb (SEQ ID NO: 1, wherein X is independently any amino acid and b is independently any positively charged amino acid), FKFEFKFE (SEQ ID NO: 14), KFQFQFE (SEQ ID NO: 15), QQRFQFQFEQQ (SEQ ID NO: 16), QQRFQWQFEQQ (SEQ ID NO: 17), FEFEFKFKFEFEFKFK (SEQ ID NO: 18), QQRFEWEFEQQ (SEQ ID NO: 19), QQXFXWXFQQQ (SEQ ID NO: 20, where X is ornithine), FKFEFKFEFKFE (SEQ ID NO: 21), FKFQFKFQFKFQ (SEQ ID NO: 22), AEAKAEAKAEAKAEAK (SEQ ID NO: 23), AEAEAKAKAEAEAKAK (SEQ ID NO: 24), AEAEAEAEAKAKAKAK (SEQ ID NO: 25), RADARADARADARADA (SEQ ID NO: 26), RARADADARARADADA (SEQ ID NO: 27), SGRGYBLGGQGAGAAAAAGGAGQGGYGGLGSQG (SEQ ID NO: 28), EWEXEXEXEX (SEQ ID NO: 29, where X is Val, Ala, Ser, or Pro), WKXKXKXKXK (SEQ ID NO: 30, where X is Val, Ala, Ser, or Pro), KWKVKVKVKVKVKVK (SEQ ID NO: 31, where X is Val, A, Ser, or Pro), LLLLKKKKKKKKLLLL (SEQ ID NO: 32), VKVKVKVKVDPPTKVKVKVKV (SEQ ID NO: 33), VKVKVKVKVDPPTKVKTKVKV (SEQ ID NO: 34), KVKVKVKVKDPPSVKVKVKVK (SEQ ID NO: 35), VKVKVKVKVDPPSKVKVKVKV (SEQ ID NO: 36), VKVKVKTKVDPPTKVKTKVKV (SEQ ID NO: 37), QQKFxFQFEQQ (SEQ ID NO: 38, wherein x is Glu, Asp, or Asn), QQKFQxQFEQQ (SEQ ID NO: 39, wherein x is Trp or Tyr), and QQKFQFxFEQQ (SEQ ID NO: 40, wherein x is Glu, Asp, or Asn).

34. The nanofiber of any one of claims 27-33, wherein the nanofiber comprises a combination of 10 to 10,000, or 100 to 10,000 anaphylatoxin conjugate peptides.

35. The nanofiber of any one of claims 27-34, wherein the nanofiber comprises a plurality of the same or different anaphylatoxin epitopes.

36. The nanofiber of any one of claims 33-35, wherein the nanofiber comprises a combination of 10 to 10,000, or 100 to 10,000 peptides comprising anaphylatoxin conjugate peptides, T-cell epitope-conjugate peptides, and plain self-assembling peptides.

37. The nanofiber of any one of claims 32-36, wherein the nanofiber comprises a combination of 10 to 10,000, or 100 to 10,000 peptides comprising anaphylatoxin conjugate peptides, T-cell epitope-conjugate peptides, PAS conjugate peptides or PEG conjugate peptides, and plain self-assembling peptides.

38. The nanofiber of any one of claims 33-37, wherein the nanofiber comprises a combination of 10 to 10,000, or 100 to 10,000 peptides comprising anaphylatoxin conjugate peptides, PAS conjugate peptides or PEG conjugate peptides, and plain self-assembling peptides.

39. The nanofiber of any one of claims 27-38, wherein at least about 1%, 5%, 10%, 15%, 20%, 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, or 97.5% of the peptides in the nanofiber are anaphylatoxin conjugate peptides.

40. The nanofiber of any one of claims 28-39, wherein at least about 1%, 2%, 3%, 4%, 5%, 6%, 7%, 8%, 9%, 10%, 11%, 12%, 13%, 14%, or 15% of the peptides in the nanofiber are T-cell epitope-conjugate peptides.

41. The nanofiber of any one of claims 28-40, wherein the anaphylatoxin conjugate peptide and the T-cell epitope-conjugate peptide are present in the nanofiber at a ratio of about 3:1, 4:1, 5:1, 6:1, 7:1, 8:1, 9:1, 10:1, 12:1, 14:1, 16:1, 18:1, 20:1, 22:1, 24:1, 26:1, 28:1, 30:1, 32:1, 34:1, 36:1, 38:1, or 40:1.

42. The nanofiber of any one of claims 27-41, wherein the self-assembling peptide forms a fibril including beta-sheet structures or a fibril having a coiled coil structure.

43. The nanofiber of any one of claims 27-41, wherein the self-assembling peptide forms a fibril having a structure of a helical filament formed around a central axis.

44. The nanofiber of claim 43, wherein the N-terminus of each self-assembling peptide is positioned at the exterior of the helical filament.

45. The nanofiber of any one of claims 27-44, wherein the anaphylatoxin epitopes are exposed on the exterior surface of the nanofiber.

46. The nanofiber of any one of claims 27-45, wherein the nanofiber is about 5-30 nm in width.

47. The nanofiber of any one of claims 27-46, wherein the nanofiber is about 100 nm to 1 μm, 100 nm to 2 μm, 100 nm to 3 μm, 100 nm to 4 μm, or 100 nm to 5 μm in length.

48. A pharmaceutical composition comprising: (a) the anaphylatoxin conjugate peptide of any one of claims 1-26 or the nanofiber of any one of claims 27-47; and (b) a pharmaceutically acceptable carrier, diluent, and/or excipient.

49. The pharmaceutical composition of claim 48, further comprising: (c) an adjuvant selected from cyclic-di-AMP, CpG, cyclic GMP-AMP (cGAMP), cholera toxin B subunit (CTB), retinoic acid, heat labile toxin B subunit, alum, MF59, 3M-052, iscomatrix, squalene-based adjuvants, AS01, AS03, or AS04, or a combination thereof.

50. The pharmaceutical composition of claim 48 or 49, formulated into a tablet.

51. The pharmaceutical composition of claim 50, wherein the tablet is a dissolving tablet.

52. A method of treating an inflammatory condition, the method comprising administering to a subject a therapeutically effective amount of the anaphylatoxin conjugate peptide of any one of claims 1-26, or the nanofiber of any one of claims 27-47, or the pharmaceutical composition of any one of claims 48-51.

53. The method of claim 52, wherein the inflammatory condition comprises inflammatory bowel disease (IBD), sepsis, ulcerative colitis, Crohn's disease, rheumatoid arthritis, psoriasis, cirrhosis, atherosclerosis, or cardiovascular inflammation, lupus, multiple sclerosis, Alzheimer's disease, pulmonary fibrosis, immunosenescence, or a combination thereof.

54. A method of reducing inflammation in a subject, the method comprising administering to the subject a therapeutically effective amount of the anaphylatoxin conjugate peptide of any one of claims 1-26, or the nanofiber of any one of claims 27-47, or the pharmaceutical composition of any one of claims 48-51.

55. The method of any one of claims 52-54, wherein the inflammation is chronic or acute.

56. A method of treating sepsis, the method comprising administering to a subject a therapeutically effective amount of the anaphylatoxin conjugate peptide of any one of claims 1-26, or the nanofiber of any one of claims 27-47, or the pharmaceutical composition of any one of claims 48-51.

57. The method of any one of claims 52-56, wherein the anaphylatoxin conjugate peptide or the nanofiber or the pharmaceutical composition is administered sublingually.

58. The method of any one of claims 52-57, wherein the subject forms an antibody to the anaphylatoxin epitope.

59. The method of claim 58, wherein the antibody comprises IgG and/or IgA.
